(19) United States
(12) Patent Application Publication
(10) Pub. No.: US 2024/0254485 A1
(43) Pub. Date: Aug. 1, 2024

(54) SIRNAS TARGETING SLC10A1 TRANSCRIPTS, COMPOSITIONS AND USES THEREOF

(71) Applicant: HemoShear Therapeutics, Inc., Charlottesville, VA (US)

(72) Inventors: Ryan Feaver, Charlottesville, VA (US); Brian Johns, Charlottesville, VA (US); Jennifer Marks, Charlottesville, VA (US); Emile Velthuisen, Charlottesville, VA (US); Maria Sol Collado, Charlottesville, VA (US); Robert Alan Figler, Charlottesville, VA (US)

(21) Appl. No.: 18/411,859

(22) Filed: Jan. 12, 2024

Related U.S. Application Data
(60) Provisional application No. 63/479,923, filed on Jan. 13, 2023.
(60) Provisional application No. 63/501,914, filed on May 12, 2023.
(60) Provisional application No. 63/592,893, filed on Oct. 24, 2023.

(51) Int. Cl. C12N 15/113; A61P 1/16; A61P 29/00

(52) U.S. Cl. CPC C12N 15/113; A61P 1/16; A61P 29/00; C12N 2310/14; C12N 2310/321; C12N 2310/322; C12N 2310/351

(57) ABSTRACT

The present invention relates to siRNA molecules targeting a SLC10A1 mRNA transcript and methods of using the siRNA compositions for treatment of a NTCP associated disorder.

### CROSS REFERENCE TO RELATED APPLICATIONS

The application claims the benefit of, and priority to U.S. Provisional Patent Application Ser. No. 63/479,923, filed on Jan. 13, 2023; U.S. Provisional Patent Application Ser. No. 63/501,914, filed on May 12, 2023; and U.S. Provisional Patent Application Ser. No. 63/592,893, filed on Oct. 24, 2023; the contents of the above referenced patent applications are incorporated herein by reference in their entireties.

### CROSS REFERENCE TO SEQUENCE LISTING

The present application is being filed with an electronically filed Sequence Listing in XML format. The sequence listing file entitled “HEM_002 US1_SL_xml” was created on Jan. 12, 2024, and is 9,905,404 bytes in size; the information in electronic format of the Sequence Listing is incorporated herein by reference in its entirety.

### BACKGROUND

Cholestatic disorders are associated with high rates of morbidity and mortality, and are the leading cause for pediatric liver transplant. Cholestatic disorders include progressive intrahepatic familial cholestasis (PFIC), Alagille syndrome (ALGS), biliary atresia (BA), primary biliary cholangitis (PBC), primary sclerosing cholangitis (PSC), intrahepatic cholestasis of pregnancy (ICP), ductal plate abnormalities, Caroli syndrome, congenital hepatic fibrosis, and bile acid synthesis defects. Treatment of these conditions typically involves supportive care for complications from these disorders, including treatment for malnutrition, pruritis, and hypertension. There are limited effective interventions to prevent progressive liver damage in these diseases.

Hepatitis D is a liver disease resulting from co-infection or superinfection with the hepatitis D virus (HDV) with hepatitis B virus (HBV). The disease pathology resulting from a HDV and HBV infection can be extremely serious leading to severe complications and a greater likelihood rapid progression to cirrhosis and liver cancer. There are limited effective interventions available for the treatment of these infections resulting in significant unmet medical need.

Sodium taurocholate co-transporting polypeptide (NTCP) is a sodium-dependent uptake transporter residing in the basolateral membrane of hepatocytes and is involved in the hepatic uptake of bile acid from blood. In addition to its transport function, NTCP is also an important entry receptor for hepatitis virus B (HBV) and HDV. The roles of NTCP make it a potential target for treating a cholestatic disorder, Hepatitis B and/or Hepatitis D in a patient. The present invention discloses that silencing and/or downregulation of expression of the gene encoding NTCP, SLC10A1, in the liver can reduce and/or inhibit NTCP mediated activities, thereby treating a liver disease, e.g., a cholestatic disorder, hepatitis B, hepatitis D, Steatotic Liver Diseases (SLD) (i.e., MAFLD (Metabolic Fatty Liver Disease) (also known as NAFLD), MASH (also known as NASH), MetALD, ALD, specific etiology SLD, and cryptogenic SLD). siRNAs specifically targeting a SLC10A1 transcript are disclosed in the present invention.

### SUMMARY

The present invention provides, among other things, siRNA molecules that specifically target a human SLC10A1 mRNA transcript for repressing the expression of a SLC10A1 transcript, therefore reducing NTCP functions in cells, particularly in hepatocytes. The siRNA molecules are specifically modified to increase its targeting efficacy and stability in vivo. The siRNA molecules of the present invention provides advanced agents for treating a NTCP associated disorder, such as a cholestatic disorder, hepatitis B and D infection and non-alcoholic fatty liver disorder.

In one aspect of the present invention, a siRNA molecule targeting a SLC10A1 mRNA transcript comprises a double-stranded region (i.e., a duplex) of 17- to 30-base pairs in length that is formed between the complementary base pairs of a sense strand sequence and an antisense strand sequence, wherein the siRNA comprises a sense strand sequence selected from the group consisting of SEQ ID NOs: 1-15 and 481-611, or an antisense strand sequence selected from the group consisting of SEQ ID NOs: 16-30 and 612-742.

In some embodiments, the double stranded region of the siRNA is 17 base pairs, 18 base pairs, 19 base pairs, 20 base pairs, 21 base pairs, 22 base pairs, 23 base pairs, 24 base pairs, or 25 base pairs in length.

In one embodiment, the double stranded region of the siRNA is 19 base pairs in length. In another embodiment, the double stranded region of the siRNA is 21 base pairs in length.

In some embodiments, the siRNA molecule comprises at least one overhang, each overhang having up to six or fewer nucleotides, e.g., 5, 4, 3, 2, or 1 nucleotides. In some examples, the siRNA has one overhang having 2 nucleotides. In other embodiments, the siRNA is blunt-ended.

In some embodiments, the siRNA molecule targets a SLC10A1 mRNA transcript from human, rhesus monkey, cynomolgus monkey, dog, pig, mouse and/or rat. In some embodiments, the siRNA molecule targets a human SLC10A1 mRNA transcript.

In some embodiments, the siRNA comprises a pair of sense and antisense strand sequences selected from the group consisting of the pairs of SEQ ID NO: 1 and SEQ ID NO: 16, SEQ ID NO: 2 and SEQ ID NO: 17, SEQ ID NO: 3 and SEQ ID NO: 18, SEQ ID NO: 4 and SEQ ID NO: 19, SEQ ID NO: 5 and SEQ ID NO: 20, SEQ ID NO: 6 and SEQ ID NO: 21, SEQ ID NO: 7 and SEQ ID NO: 22, SEQ ID NO: 8 and SEQ ID NO: 23, SEQ ID NO: 9 and SEQ ID NO: 24, SEQ ID NO: 10 and SEQ ID NO: 25, SEQ ID NO: 11 and SEQ ID NO: 26, SEQ ID NO: 12 and SEQ ID NO: 27, SEQ ID NO: 13 and SEQ ID NO: 28, SEQ ID NO: 14 and SEQ ID NO: 29, and SEQ ID NO: 15 and SEQ ID NO: 30.

In some embodiments, the sequence of the siRNA molecule is 100% complementary to a portion of the sequence of the SLC10A1 mRNA transcript, e.g., a human SLC10A1 mRNA transcript. In some embodiments, the sequence of the siRNA molecule has a mismatch of 4 base pairs or fewer to the sequence of the SLC10A1 mRNA transcript, or a mismatch of 3 base pairs or fewer to the sequence of the SLC10A1 mRNA transcript, or a mismatch of 2 base pairs to the sequence of the SLC10A1 mRNA transcript, or a mismatch of 1 base pairs to the sequence of the SLC10A1 mRNA transcript.

In some embodiments, the sense and/or antisense sequence of the siRNA is modified, for example the first nucleotide of the antisense strand is modified to be Uracil and/or the last nucleotide of the sense strand is modified to be Adenine. For example, the siRNA may comprise the sense strand sequence selected from the group consisting to SEQ ID NOs: 31-45. In some cases, the siRNA may comprise the antisense strand sequence selected from the group consisting to SEQ ID NOs: 46-60.

In some embodiments, the siRNA comprises a pair of sense and antisense strand sequences selected from the group consisting of the pairs of SEQ ID NO: 31 and SEQ ID NO: 46, SEQ ID NO: 32 and SEQ ID NO: 47, SEQ ID NO: 33 and SEQ ID NO: 48, SEQ ID NO: 34 and SEQ ID NO: 49, SEQ ID NO: 35 and SEQ ID NO: 50, SEQ ID NO: 36 and SEQ ID NO: 51, SEQ ID NO: 37 and SEQ ID NO: 52, SEQ ID NO: 38 and SEQ ID NO: 53, SEQ ID NO: 39 and SEQ ID NO: 54, SEQ ID NO: 40 and SEQ ID NO: 55, SEQ ID NO: 41 and SEQ ID NO: 56, SEQ ID NO: 42 and SEQ ID NO: 57, SEQ ID NO: 43 and SEQ ID NO: 58, SEQ ID NO: 44 and SEQ ID NO: 59, and SEQ ID NO: 45 and SEQ ID NO: 60.

In some embodiments, the siRNA comprises one or more chemical modifications, e.g., 2′-O-methyl modification, 2′-fluoro modification, introduction of phosphorothioate modified nucleotides, substitution of uracil with 5-Propynyluracil, substitution of uracil with 5′-methyluridine, substitution of uracyl ribose nucleotides with 4′-thioribose and substitution of uracyl ribose nucleotides with deoxythymidine nucleotides and combinations thereof. In some embodiments, the siRNA comprises combined modifications of 2′-fluoro, 2′-OMe, phosphorothioate and terminal vinyl-phosphonate 2′-OMe modifications.

In some embodiments, the siRNA further comprises one or more N-Acetylgalactosamine (GalNAc) moiety, or derivative thereof. In some embodiments, the GalNAc moiety is conjugated to a terminus of the siRNA molecule. In some examples, the GalNAc moiety is conjugated to the terminus of the sense strand, e.g., the 3′ end of the sense strand of the siRNA.

In some embodiments, the siRNA targeting a SLC10A1 mRNA transcript does not comprise the GalNAc moiety.

In some embodiments, the siRNA comprises a specific modification pattern.

In some embodiments, the siRNA comprises a modified sense strand sequence selected from the group consisting of SEQ ID NOs: 61-270, or a modified antisense strand sequence selected from the group consisting of SEQ ID NOs: 271-480. As non-limiting examples, the siRNA may comprise a pair of the modified sense and antisense strand sequences in Table 3.

In addition, the present invention provides a siRNA molecule comprising a sense strand having a sequence of any one of SEQ ID NOs: 481-611, or an antisense strand having a sequence of any one of SEQ ID NOs: 612-742. As non-limiting examples, the siRNA may comprise a pair of sense and antisense strand sequences in Table 4.

In some embodiments, the siRNA molecule comprises a modified sense strand having a sequence of any one of SEQ ID NOs: 743-873. In some embodiments, the siRNA molecule comprises a modified antisense strand having a sequence of any one of SEQ ID NOs: 874-1004. As non-limiting examples, the siRNA may comprise a pair of sense and antisense strand sequences in Table 5.

In another aspect, the present invention provides a pharmaceutical composition comprising one or more siRNA molecules described herein, and a pharmaceutically acceptable excipient or carrier. The composition may be formulated specifically for any administration routes. In some embodiments, the composition may comprise two or more siRNA molecules having different pairs of sense and antisense strands described in the present disclosure, for example, two siRNA molecules targeting different parts of the human SLC10A1 mRNA transcript, e.g., the 5′ non-coding sequence, coding sequence and/or the 3′ non-coding sequence.

In some embodiments, the siRNA molecule described herein, and the composition thereof may be of use for manufacture of a medication for treating a NTCP associated disorder, such as cholestatic disorder, hepatitis B, hepatitis D, MAFLD, MASH, or MetALD.

In some embodiments, the siRNA molecule described herein, and the composition thereof may be of use for manufacture of a medication for treating cholestasis caused by another disease or procedure, such as cholestasis after liver transplantation or cholestasis from a malignant biliary obstruction.

In some embodiments, the siRNA molecule described herein, and the composition thereof may be of use for manufacture of a medication for diabetes, obesity, dyslipidemia, inflammatory bowel disease, or constipation.

In another aspect of the present disclosure, provided also includes a method of treating a cholestatic disorder in a patient in need; the method comprises administering to the patient the siRNA molecule described herein, or the composition thereof. The cholestatic disorder may be progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP). In some embodiments, cholestasis may be caused by another disease or procedure, such as cholestasis after liver transplantation or cholestasis from a malignant biliary obstruction. In some embodiments, the patient is a pediatric patient. In some embodiments, the patient is an adult.

Treatment with the siRNA molecule described herein, or the composition thereof may reduce intrahepatic accumulation of bile acids in the treated patient. The bile acid accumulation may be reduced about 10%, 15%, 20%, 25%, 30%, 35%, 40%, 45%, 50%, 55%, 60%, 65%, 70%, 75%, 80%, 85%, 90%, 95% or 100% as compared to the concentration of bile acid before the treatment. The reduction may be due to the impaired NTCP mediated bile acid uptake.

In accordance, after treating, the patient experiences an improvement in at least one symptom of a cholestatic disorder, selected from the group consisting of pruritis, mitochondrial damage and inflammation in the liver, liver function tests, and hepatic injury.

In another aspect of the present disclosure, provided also includes a method of treating hepatitis B and/or hepatitis D in a patient in need; the method comprises administering to the patient the siRNA molecule described herein, or the composition thereof. In some embodiments, the patient is tested as hepatitis B or D antigen positive. In some embodiments, the patient is tested as hepatitis B or D antigen negative.

In another aspect of the present disclosure, provided also includes a method of curing hepatitis B and/or hepatitis D in a patient in need; the method comprises administering to the patient the siRNA molecule described herein, or the composition thereof. In some embodiments, the patient is tested as hepatitis B or D antigen positive. In some embodiments, the patient is tested as hepatitis B or D antigen negative.

In some embodiments, the siRNA described herein or the composition thereof is used as a monotherapy or in a regimen combination with one or more antiviral agents. The antiviral agents may include but are not limited to interferons, nucleoside and nucleotide analogs that interfere with DNA polymerases, siRNA drugs that target part of the viral genome, viral entry inhibitors, viral capsid or core inhibitors, S-antigen inhibitors, antisense molecules that target the viral mRNA, gene editing agents, therapeutic vaccines, monoclonal antibodies that neutralize HBV or HDV proteins, and prenylation inhibitors.

In another embodiment, the siRNA described herein or the composition thereof is used optionally with additional antiviral agents in combination with immunomodulators. The immunomodulatory agents include but are not limited to therapeutic vaccines, monoclonal antibodies that neutralize HBV proteins with or without FC domain signaling capabilities, check point inhibitors, immune system adjuvants and modulatory agents including potentially cell therapies.

In some embodiments, the siRNA molecule described herein, and the composition thereof may be of use for manufacture of a medication for cancer. In embodiments, hepatocellular carcinoma (HCC) is caused by hepatitis (e.g., hepatitis B such as chronic hepatitis B or hepatitis D such as chronic hepatitis D). In accordance, the present invention also provides a method for treating a cancer caused by hepatitis in a subject in need; the method comprises administering to the subject in need an effective amount of the siRNA described herein, alone or in combination with another anti-cancer therapy.

In yet another aspect, the present disclosure provides a method of treating metabolic dysfunction-associated steatotic liver disease (MASLD) and metabolic dysfunction-associated steatohepatitis (MASH) in a patient in need; the method comprises administering to the patient the siRNA molecule described herein, or the composition thereof.

### BRIEF DESCRIPTION OF DRAWINGS

FIG. 1A illustrates use of an exemplary siRNA (duplex ID=SR122) to assess the serum bile acid (sBA) response when administered at a single dose of 3 mg/kg (3 mpk) or at 10 mg/kg (10 mpk). At the 3 mpk dose, a durable response is observed for 90 days. At the 10 mpk dose, a durable response is observed for 120 days.

FIG. 1B illustrates use of an exemplary siRNA (duplex ID=SR122) to assess the impact of SLC10A1 gene expression using a control, 3 mpk, and 10 mpk doses. At days 28, 60, and 90, the 10 mpk dose is illustrated on the left and the 3 mpk is illustrated on the right. The 10 mpk dose was further assessed on days 120 and 150. Robust gene knockdown was observed at both doses.

FIG. 1C illustrates use of an exemplary siRNA (duplex ID=SR122) in liver function tests (LFTs) over the course of the 10 mpk study as assessed at 0, 21, 45, 65, and 119 days for (left to right) ALT, ALP, AST, and TBIL. As illustrated in this figure, LFTs remain unchanged from baseline values.

FIG. 2 illustrates use of an exemplary siRNA (duplex ID=SR319) as a mouse NTCP-targeting surrogate siRNA for in vivo studies.

FIG. 3 illustrates a study design for a seven-day bile-duct ligation (BDL) mouse model to study an exemplary siRNA (duplex ID=SR319) and an exemplary small molecule (“SMOL”).

FIG. 4 illustrates that inhibiting NTCP with an exemplary siRNA (duplex ID=SR319) or an exemplary small molecule (“SMOL”) increases the concentration of serum taurocholic acid (TCA), a bile acid, when compared to BDL+Vehicle.

FIG. 5 illustrates that inhibiting NTCP with an exemplary siRNA (duplex ID=SR319) or an exemplary small molecule (“SMOL”) reduces serum ALP and ALT at days 3 and 7. For a given day, the bars as read from left to right relate to data for: Sham+Vehicle; BDL+Vehicle; BDL+SMOL; and BDL+SR319.

FIG. 6 illustrates that inhibiting NTCP with an exemplary siRNA (duplex ID=SR319) or an exemplary small molecule (“SMOL”) reduces inflammatory, fibrotic and proliferative markers.

FIG. 7 illustrates that an exemplary siRNA (duplex ID=SR319) or an exemplary small molecule (“SMOL”) each lower intracellular bile acids as evidenced by reduced FXR signaling.

### DETAILED DESCRIPTION

### Definitions

The terminology used herein is for the purpose of describing particular embodiments only and is not intended to be limiting.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and or the claims, such terms are intended to be inclusive in a manner similar to the term “comprising.”

The term “about” or “approximately” means within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, and up to 1% of a given value. Alternatively, particularly with respect to biological systems or processes, the term can mean within an order of magnitude, e.g., within 5-fold, or within 2-fold, of a value. Where particular values are described in the application and claims, unless otherwise stated the term “about” meaning within an acceptable error range for the particular value should be assumed.

Complementary: As used herein, the term “complementary” refers to the ability of polynucleotides to form base pairs with one another. Base pairs are typically formed by hydrogen bonds between nucleotide units in antiparallel polynucleotide strands. Complementary polynucleotide strands can base pair in the Watson-Crick manner (e.g., A to T, A to U, C to G), or in any other manner that allows for the formation of duplexes. As persons skilled in the art are aware, when using RNA as opposed to DNA, uracil rather than thymine is the base that is considered to be complementary to adenosine. However, when a U is denoted in the context of the present invention, the ability to substitute a T is implied, unless otherwise stated. “100% complementarity” or “100% complementary to” refers to the situation in which each nucleotide unit of one polynucleotide strand can hydrogen bond with a nucleotide unit of a second polynucleotide strand. Less than perfect complementarity refers to the situation in which some, but not all, nucleotide units of two strands can hydrogen bond with each other. For example, for two 20-mers, if only two base pairs on each strand can hydrogen bond with each other, the polynucleotide strands exhibit 10% complementarity. In the same example, if 18 base pairs on each strand can hydrogen bond with each other, the polynucleotide strands exhibit 90% complementarity.

Effective amount: As used herein, the term “effective amount” is meant the amount required to reduce or improve at least one symptom of a disease relative to an untreated patient. The effective amount of active compound(s) used to practice the present invention for therapeutic treatment of a disease varies depending upon the manner of administration, the age, body weight, and general health of the subject.

mRNA: As used herein, the term “mRNA” refers to a nucleic acid transcribed from a gene from which a polypeptide is translated, and may include non-translated regions such as a 5′UTR and/or a 3′UTR. It will be understood that a siRNA of the invention may comprise a nucleotide sequence that is complementary to any sequence of an mRNA molecule, including translated regions, the 5′UTR, the 3 UTR, and sequences that include both a translated region and a portion of either 5′UTR or 3′UTR. A siRNA of the invention may comprise a nucleotide sequence that is complementary to a region of an mRNA molecule spanning the start codon or the stop codon. The term “mRNA transcript” refers to the product resulting from RNA polymerase catalyzed transcription of a DNA sequence. When the RNA transcript is a perfect complementary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

Nucleotide: As used herein, the term “nucleotide” refers to a ribonucleotide or a deoxyribonucleotide or modified form thereof, as well as an analog thereof. Nucleotides include species that comprise purines, e.g., adenine, hypoxanthine, guanine, and their derivatives and analogs, as well as pyrimidines, e.g., cytosine, uracil, thymine, and their derivatives and analogs. Nucleotide analogs include nucleotides having modifications in the chemical structure of the base, sugar and/or phosphate, including, but not limited to, 5-position pyrimidine modifications, 8-position purine modifications, modifications at cytosine exocyclic amines, and substitution of 5-bromo-uracil; and 2′-position sugar modifications, including but not limited to, sugar-modified ribonucleotides in which the 2′-OH is replaced by a group such as an H, OR, R, halo, SH, SR, NH₂, NHR, NR₂, or CN, wherein R is an alkyl moiety. Nucleotide analogs are also meant to include nucleotides with bases such as inosine, queuosine, xanthine, sugars such as 2′-methyl ribose, non-natural phosphodiester linkages such as methylphosphonates, phosphorothioates and peptides. The term nucleotide is also meant to include what are known in the art as universal bases. By way of example, universal bases include but are not limited to 3-nitropyrrole, 5-nitroindole, or nebularine. The term “nucleotide” is also meant to include the N3′ to P5′ phosphoramidate, resulting from the substitution of a ribosyl 3′ oxygen with an amine group.

Pharmaceutically acceptable: As used herein, the term “pharmaceutically acceptable” refers to those properties and/or substances that are acceptable to the patient from a pharmacological/toxicological point of view and to the manufacturing pharmaceutical chemist from a physical/chemical point of view regarding composition, formulation, stability, patient acceptance and bioavailability. “Pharmaceutically acceptable carrier” refers to a medium that does not interfere with the effectiveness of the biological activity of the active ingredient(s) and is not toxic to the host to which it is administered. The term “pharmaceutical composition” or “pharmaceutically acceptable composition” refers to a mixture of at least one compound or molecule useful within the invention with a pharmaceutically acceptable carrier. The pharmaceutical composition facilitates administration of the compound or molecule to a patient. Multiple techniques of administering a compound or molecule exist in the art including, but not limited to, intravenous, subcutaneous, oral, aerosol, parenteral, ophthalmic, pulmonary and topical administration. As used herein, the term “pharmaceutically acceptable carrier” means a pharmaceutically acceptable material, composition or carrier, such as a liquid or solid filler, stabilizer, dispersing agent, suspending agent, diluent, excipient, thickening agent, solvent or encapsulating material, involved in carrying or transporting a compound or molecule useful within the invention within or to the patient such that it may perform its intended function. Typically, such constructs are carried or transported from one organ, or portion of the body, to another organ, or portion of the body. Each carrier must be “acceptable” in the sense of being compatible with the other ingredients of the formulation, including the compound useful within the invention, and not injurious to the patient. Some examples of materials that may serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; surface active agents; alginic acid; pyrogen-free water; isotonic saline; Ringer's solution; ethyl alcohol; phosphate buffer solutions; and other non-toxic compatible substances employed in pharmaceutical formulations. As used herein, “pharmaceutically acceptable carrier” also includes any and all coatings, antibacterial and antifungal agents, and absorption delaying agents, and the like that are compatible with the activity of the compound useful within the invention, and are physiologically acceptable to the patient. Supplementary active compounds may also be incorporated into the compositions. The “pharmaceutically acceptable carrier” may further include a pharmaceutically acceptable salt of the compound or molecule useful within the invention. Other additional ingredients that may be included in the pharmaceutical compositions used in the practice of the invention are known in the art and described, for example in Remington's Pharmaceutical Sciences (Genaro, Ed., Mack Publishing Co., 1985, Easton, Pa.), which is incorporated herein by reference.

Prevent: As used herein, the terms “prevent,” “preventing,” “prevention,” and the like refer to reducing the probability of developing a disorder or condition in a subject, who does not have, but is at risk of or susceptible to developing a disorder or condition.

Reduce: As used herein, the terms “reduce” or “reducing” or “reduction” refer to silencing, eliminating, knock-down, knock-out, and/or decreasing expression of a target gene. The term “reduced” is used herein to indicate that the target gene expression is lowered by 1-100%, or 10-100%, 20-80%, 30-80%, or 40-60%. For example, the expression may be reduced by at least 10%, at least 20%, at least 30%, at least 40%, at least 50%, at least 60%, at least 70%, at least 80%, at least 90%, at least 95%, or even 99% knock-down of gene expression can be directed by the use of siRNAs or other interfering nucleic acids.

siRNA: As used herein, the terms “small interfering” or “short interfering RNA” or “siRNA” is a RNA duplex of nucleotides that is targeted to a gene interest. A “RNA duplex” refers to the double-stranded region formed by the complementary pairing between two sense and anti-sense regions of a RNA molecule. siRNA is “targeted” to a gene in that the nucleotide sequence of the duplex portion of the siRNA is complementary to a nucleotide sequence of the targeted gene. In some embodiments, the length of the duplex (i.e., the double stranded region) of siRNAs is less than 30 nucleotides. The duplex can be 29, 28, 27, 26, 25, 24, 23, 22, 21, 20, 19, 18, 17, 16, 15, 14, 13, 12, 11 or 10 nucleotides in length. In general, the length of the duplex can be 19, 21, or 23 nucleotides in length.

Subject: As used herein, the term “subject” or “patient,” may be a human or non-human mammal. Non-human mammals include, for example, livestock and pets, such as ovine, bovine, porcine, canine, feline and murine mammals. Preferably, the subject is human.

Target gene: As used herein, “target gene” refers to a nucleic acid sequence in a cell, wherein the expression of the sequence may be specifically and effectively modulated using siRNAs and methods described herein. A “gene” is used broadly to refer to any segment of nucleic acid associated with a biological function. Thus, genes include coding sequences and/or the regulatory sequences required for their expression. For example, “gene” refers to a nucleic acid fragment that expresses mRNA, functional RNA, or specific protein, including regulatory sequences. “Genes” also include nonexpressed DNA segments that, for example, form recognition sequences for other proteins. “Genes” can be obtained from a variety of sources, including cloning from a source of interest or synthesizing from known or predicted sequence information, and may include sequences designed to have desired parameters.

Therapeutically-effective amount: As used herein, the term “therapeutically-effective amount” refers to an amount of a compound, material, or composition comprising an oligonucleotide herein which is effective for producing some desired therapeutic effect in at least a sub-population of cells in an animal at a reasonable benefit/risk ratio applicable to any medical treatment.

Treat: The terms “treatment,” “treating,” “treat” and the like, when used in the context of a disease, injury or disorder, are used herein to generally mean obtaining a desired pharmacologic and/or physiologic effect, and may also be used to refer to improving, alleviating, and/or decreasing the severity of one or more symptoms of a condition being treated. The effect may be prophylactic in terms of completely or partially delaying the onset or recurrence of a disease, condition, or symptoms thereof, and/or may be therapeutic in terms of a partial or complete cure for a disease or condition and/or adverse effect attributable to the disease or condition. “Treatment” as used herein covers any treatment of a disease or condition of a mammal, particularly a human, and includes: (a) preventing the disease or condition from occurring in a subject which may be predisposed to the disease or condition but has not yet been diagnosed as having it; (b) inhibiting the disease or condition (e.g., arresting its development); or (c) relieving the disease or condition (e.g., causing regression of the disease or condition, providing improvement in one or more symptoms).

### NTCP and Biological Functions

Na⁺-taurocholate Co-transporting Polypeptide (NTCP) is a hepatic uptake transporter in the basolateral membrane of the hepatocyte to uptake bile acids from plasma, playing a crucial role in the enterohepatic circulation of bile acids (Hagenbuch and Dawson, 2004, Pflugers Arch. 447, 566-570). Bile acids are the catabolic product of cholesterol metabolism; hence this protein is important for cholesterol homeostasis. In addition to their role in metabolism, bile acids also act as transcriptional regulators via certain nuclear receptors (NRs). For example, bile acids are strong activators (ligands) for FXR signaling pathway, which plays an important role in balancing bile acids in the liver.

Increased intracellular accumulation of bile acids ultimately results in bile acid-induced hepatocellular damage and apoptosis. NTCP is a key player in this coordinated response designed to help shield the hepatocyte from bile acid damage. The key role of NTCP in cholestasis makes it a superior target for treating a cholestatic disorder.

NTCP is also a functional entry receptor for hepatitis B virus (HBV) and hepatitis D virus (HDV). HBV as well as HDV attach with low-affinity to heparan sulfate proteoglycans on the surfaces of hepatocytes. After this initial attachment, both viruses bind to NTCP through a high-affinity interaction followed by internalization of the viral particles. It has been shown that preventing interaction between this cell surface receptor and HBV or HDV viral particle can inhibit HBV and HDV infection. For example, Yan et al. (Down-regulation of cell membrane localized NTCP expression in proliferating hepatocytes prevents hepatitis B virus infection, Emerg, Microbes Infect. 2019, 8(1): 879-894) reported that primary human hepatocytes with downregulates NTCP result in a reduced or even complete loss of HBV infection capacity. As NTCP plays an essential role in in HBV and HDV infection cycle, NTCP has been exploited as a potential target to develop new antiviral therapies.

NTCP has a pivotal role in bile acid dynamics, bile acid signaling and in viral infection of the liver.

NTCP in humans is encoded by the gene SLC10A1 (solute carrier family 10 (sodium/bile acid cotransporter) member 1), which is mainly expressed in the liver. Human SLC10A1 mRNA (Gene Bank Ref. No. NM_003049.4) comprises the sequence of

GATTAAAGAAGGCATCCAGCAAGAACTGCACAAGAAACGGAGTCAGCCGG
AGAACAAGGAGTGGTCTTCCACTGCCTCACAGGAGGATGGAGGCCCACAA
CGCGTCTGCCCCATTCAACTTCACCCTGCCACCCAACTTTGGCAAGCGCC
CCACAGACCTGGCACTGAGCGTCATCCTGGTGTTCATGTTGTTCTTCATC
ATGCTCTCGCTGGGCTGCACCATGGAGTTCAGCAAGATCAAGGCTCACTT
ATGGAAGCCTAAAGGGCTGGCCATCGCCCTGGTGGCACAGTATGGCATCA
TGCCCCTCACGGCCTTTGTGCTGGGCAAGGTCTTCCGGCTGAAGAACATT
GAGGCACTGGCCATCTTGGTCTGTGGCTGCTCACCTGGAGGGAACCTGTC
CAATGTCTTCAGTCTGGCCATGAAGGGGGACATGAACCTCAGCATTGTGA
TGACCACCTGCTCCACCTTCTGTGCCCTTGGCATGATGCCTCTCCTCCTG
TACATCTACTCCAGGGGGATCTATGATGGGGACCTGAAGGACAAGGTGCC
CTATAAAGGCATCGTGATATCACTGGTCCTGGTTCTCATTCCTTGCACCA
TAGGGATCGTCCTCAAATCCAAACGGCCACAATACATGCGCTATGTCATC
AAGGGAGGGATGATCATCATTCTCTTGTGCAGTGTGGCCGTCACAGTTCT
CTCTGCCATCAATGTGGGGAAGAGCATCATGTTTGCCATGACACCACTCT
TGATTGCCACCTCCTCCCTGATGCCTTTTATTGGCTTTCTGCTGGGTTAT
GTTCTCTCTGCTCTCTTCTGCCTCAATGGACGGTGCAGACGCACTGTCAG
CATGGAGACTGGATGCCAAAATGTCCAACTCTGTTCCACCATCCTCAATG
TGGCCTTTCCACCTGAAGTCATTGGACCACTTTTCTTCTTTCCCCTCCTC
TACATGATTTTCCAGCTTGGAGAAGGGCTTCTCCTCATTGCCATATTTTG
GTGCTATGAGAAATTCAAGACTCCCAAGGATAAAACAAAAATGATCTACA
CAGCTGCCACAACTGAAGAAACAATTCCAGGAGCTCTGGGAAATGGCACC
TACAAAGGGGAGGACTGCTCCCCTTGCACAGCCTAGCCCTTCCCCTGGTG
GCCTGGATTCTGGTCCCAAAGCAATTCTGAAAGCCAGTGTGGTAAACTAG
AGAGAGCAGCAAAAACACCAGTCTTGCCTGAGTCTTTCTCCAGCATTTCC
AGTACATCTATCAGAATCATCAAGTCTTGGCCGGGAACACAGACAGGGTG
TCTACCCAAGAAGCCTCACCTATCCCCAACTTAGAATTTGCTACTTATTT
TAAAGACTTGTTCAGTGACTGTAAACTCTATGAAACCAGAAACCGAATCT
GCCTCTTGCTGGGATCTCTAAAAGTGTCTGATAAGCATCTTAAAGTCACT
CAATTCCTGAACTAATCAATATATATGTTTAACCCATTACTCAAATACCC
AAATCCCATTCCAAGTTTTGTGACCCAAAAGAGAAATAAATGCTCACAAG
TGCTGTAGAATTAAACTTCAGAAGTTCTAACCTAAAAAGTTCAGATCCTA
TTCCTTCCCTTTTGACATTATTGGGATGATGCTCCCGAAAAGTCAAATTT
GACATCAAGTATGCAAAAGTGAACACAGTAAGATGCAATCAGGCAAAACA
AACTCAAAAAATAGCTAATGAAATGAAAAAACTGGGCGAATGCATCATGT
TAGTAGAGGAGGAAAACTTTTGACAAGGAAAAACCAGGAAACAAACACAT
ACATTAACACAATGTTACCTCACTAATAATCTCTTTTTTAAGTTCAGTAG
GTATTTGGTTTTAACACAAGGCGCCCCCAAAACTGGGTACAGCAAACTAC
TGCCAAAATGAGTCGTTTCCCAATTCAAAGAAAAATGTTCAAATACCTAG
AATTCACAAATTTCAAATTGTCTCTAATAAAATTTAAACATTTTGCATAT
CA
(GeneBank Ref. No. NM_003049. 4; SEQ ID NO: 1093)

siRNA Molecules Inhibiting the Expression of a SLC1041 mRNA Transcript

In one aspect of the present disclosure, provided included a small interfering RNA (siRNA) molecule that is capable of inhibiting the expression of a SLC10A1 mRNA transcript, particularly a human SLC10A1 mRNA transcript.

siRNA Molecules

Small interfering RNAs (siRNAs) can activate the RNAi pathway to inhibit the expression of genes through post-transcriptional gene silencing. siRNA has innate advantages over small molecular therapeutics and monoclonal antibody drugs because siRNA executes its function by complete Watson-Crick base pairing with mRNA, whereas small molecule and monoclonal antibody drugs need to recognize the complicated spatial conformation of certain proteins.

A siRNA is a double-stranded RNA (dsRNA) molecule comprising a sense strand and an antisense strand, in which the sense strand and antisense strand sequences form a double-stranded region (i.e., a duplex region) by the Watson-Crick base pairing interactions between the complementary base pairs of the sense and antisense strands of the siRNA. The identity of duplex is confirmed by melting temperature (Tm). The double stranded region (i.e., the duplex) of the siRNA may comprise 15-30 base pairs, or 17-28 base pairs, 17-25 base pairs, 21-23 base pairs, or 19-23 base pairs. As non-limiting examples, the double stranded region of the siRNA comprises 17 base pairs, 18 base pairs, 19 base pairs, 20 base pairs, 21 base pairs, 22 base pairs, 23 base pairs, 24 base pairs, or 25 base pairs. In one embodiment, the double stranded region of the siRNA molecule comprises 19 base pairs. In another embodiment, the double stranded region of the siRNA molecule comprises 21 base pairs.

siRNA exerts its effect at the post-transcriptional level. This siRNA duplex arises when long dsRNA is cleaved by Dicer, a member of the RNAse III family. The antisense strand of the duplex acts as a guide strand to incorporate the siRNA into RNA-induced silencing complex (RISC) where it interacts with its Argouate 2 component, resulting in duplex unwinding and degradation of the passenger strand (i.e., the sense strand). The antisense strand, which is complementary to the target mRNA, then guides the RISC complex to the mRNA, leading downregulation of the mRNA. siRNA only functions when its antisense strand sequence is completely base-paired to a sequence of the target mRNA. In some cases, a few mismatches are tolerated by the RNA-induced silencing complex (RISC) as well.

In accordance, the siRNA molecule of the present disclosure comprises a sequence complementary to a portion of the sequence of a SLC10A1 mRNA transcript. In some embodiments, the siRNA molecule has a mismatch of 4 base pairs or fewer to the portion of the sequence of the SLC10A1 mRNA transcript. As a non-limiting example, the siRNA molecule has a mismatch of 3 base pairs to the portion of the sequence of the SLC10A1 mRNA transcript. In another example, the siRNA molecule has a mismatch of 2 base pairs to the portion of the sequence of the SLC10A1 mRNA transcript. In another example, the siRNA molecule has a mismatch of 1 base pair to the portion of the sequence of the SLC10A1 mRNA transcript.

siRNA Modifications

To maximize the treatment potency and reduce or avoid the side effects of siRNA, a siRNA molecule may comprise one or more chemical modifications. Chemically modified nucleotides can increase siRNA efficacy, specificity, and stability, reduce its toxicity and immunogenicity, and decrease off-target effects. Chemically modified siRNAs, such as siRNAs with substitution of the 2′-OH with a 2′-O-methyl (2′-OMe) or 2′-methoxyethyl (2′-MOE) group or the substitution of certain nucleotides with locked nucleic acid (LNA), unlocked nucleic acid (UNA) and glycol nucleic acid (GNA) can efficiently suppress immunostimulatory siRNA-driven innate immune activation, enhance activity and specificity, and reduce off-target-induced toxicity.

According to the natural structure of nucleotides, chemical modifications can be placed at the phosphate backbone, the ribose moiety or the base. Typically, these modifications are simultaneously introduced in siRNA, e.g., the combination of 2′-OMe and phosphorothioate (PS) modifications and the combination of 2′-OMe and 2′-deoxy-2′-fluoro (2′-F).

In some embodiments, the siRNA modifications comprise one or more phosphonate modifications of the backbone of the siRNA.

In one embodiment, the phosphorothioate (PS) linkage is introduced in the siRNA molecule described herein. This modification was achieved by leveraging a sulfur atom to replace one nonbridging oxygen of a phosphodiester. PS linkage endows modified nucleic acids with resistance to nucleases and PS modified siRNAs can more readily combine with plasma proteins, such as albumin. The position and number of PS linkages vary. In some embodiments, the sense strand comprises at least 1, 2, 3, 4, 5, 6, 7, 8, 9, 10, 11, 12, 13, 14, 15, 16, 17, 18, 19, 20, 21, 22 or 23 of PS linkages. In some embodiments, the antisense strand comprises at least 1, 2, 3, 4, 5, 6, 7, 8, 9, 10, 11, 12, 13, 14, 15, 16, 17, 18, 19, 20, 21, 22 or 23 of PS linkages. In some embodiments, both the sense strand and the antisense strand of a siRNA comprise PS linkages.

In some embodiments, the PS linkages position at the 3′ end of the sense strand sequence. In some embodiments, the PS linkages position at the 3′ end of the antisense strand sequence. In some embodiments, the PS linkages position at the 5′ end of the sense strand sequence. In some embodiments, the PS linkages position at the 5′ end of the antisense strand sequence. In some embodiments, the PS linkages position at the 3′ and 5′ ends of the sense strand sequence. In some embodiments, the PS linkages position at the 3′ and 5′ ends of the antisense strand sequence. In some embodiments, the PS linkages position at various positions of the sense strand sequence. In some embodiments, the PS linkages position at various positions of the antisense strand sequence.

In addition, other residues used to replace the phosphodiester group in the siRNA, including but not limited, phosphorodithioate (PS2), methylphosphonate (MP), methoxypropylphosphonate (MOP) and peptide nucleic acid (PNA).

In some embodiments, the siRNA described herein comprises a phosphonate modification with various analogs at the 5′-end of the siRNA. The 5′-phosphate of the siRNA is required for RISC loading. A 5′-phosphate can be modified via either phosphorylation in cells or by chemical synthesis. In some embodiments, an analog that have similar conformation and steroidal electronic properties to natural phosphates but is resistant to dephosphorylases. As non-limiting examples, the analog is 5′-(E)-vinyl phosphonate (5′-(E)-VP), 5′-methylphosphonate, (S)-5′-C-methyl with phosphate, or 5′-phosphorothioate. In some embodiments, 5′-(E)-VP decoration, which substitutes bridge oxygen and carbon with E-vinyl phosphonate moieties at the 5′-end, is the most potent and metabolically stable mechanism. The replaced hydrophobic substitutions enhance the stability of intact siRNAs and are favorable for RISC loading.

In some embodiments, the siRNA described herein may further comprise inverted deoxythymine (idT) at the strand terminus and/or termini, including UNA and X (without nucleoside base), flanking the UAU or UAUAU motif(s).

In some embodiments, the siRNA described herein comprises one or more ribose modifications.

In some embodiments, the siRNA described herein comprises one or more ribose modification at the 2′ position. Notably, 2′-O-methyl (2′-OMe) is the most frequently used modification in nucleic acid based drug. 2′-OMe modification can enhance stability of the siRNA by blocking the nucleophilic 2′-OH group. In other embodiments, other modifications at the 2′ position include 2′-O-methoxyethyl (2′-O-MOE), 2′-deoxy-2′-fluoro (2′-F), 2′-arabino-fluoro (2′-Ara-F), 2′-O-benzyl and 2′-O-methyl-4-pyridine (2′-O—CH2Py(4)).

In addition, 2′-C, 4′-C, and the whole sugar ring may be modified, resulting in ribose analogs including UNAs, LNAs, GNAs (glycol nucleic acids), (S)-cEt-BNAs, tricyclo-DNA (tcDNA) and phosphorodiamidate morpholino oligomers (PMOs). Without wishing to be bound by any theory, UNA, with higher flexibility and thermal destabilization than the unmodified product due to unconnected 2′ and 3′ carbons, can block the entry of passenger strands and promote the RISC loading of the guide strand by introducing chemical asymmetry into duplex siRNAs. GNA can be used to erase off-target effect-induced hepatotoxicity by including it in the seed region of the siRNA guide strand. LNA is a bicyclic structure that contains a bridge between the 2′ oxygen and the 4′ carbon. It “locks” the ribose into its preferred C3′-endo conformation and significantly increases the affinity of base pairing. In some embodiments, the modifications may comprise one or more bicyclic and tricyclic analogs, including ethyl-bridged nucleic acids (ENAs), constrained ethyl (cEt) nucleic acids and tricyclo-DNA.

In some embodiments, the siRNA described herein comprises one or more base modifications.

Base replacement is of great benefit to nucleic acid-based drugs. In some embodiments, the siRNA described herein comprises one or more base modification and/or base analogs. The base analogs include but are not limited to, pseudouridine, 2-thiouridine, N6-methyladenosine, 5-methylcytidine, N-ethylpiperidine triazole-modified adenosine analogs, N-ethylpiperidine 7-EAA triazole, 6′-phenylpyrrolocytosine (PhpC), uridine substitution of 2,4-difluorotolylribonucleoside (rF) and 5-fluoro-2′-deoxyuridine (FdU). Moreover, many other less common base analogs may be used in siRNA modifications.

The effects of various modifications on siRNA activity, specificity and toxicity vary; different modification patterns of siRNAs are tested clinically. In some embodiments, a universal modification pattern (e.g., standard template chemistry (STC)) is applied to the siRNA described herein. As a non-limiting example, when the lengths of the sense and the antisense of a siRNA are 21 and 23 nt, respectively, 2 PS linkages are placed at the 3′ terminus of the antisense strand, three consecutive 2′-F moieties are incorporated at positions 9, 10 and 11 of the sense strand from the 5′-end, and consecutive 2′-OMe modifications are placed at positions 11, 12 and 13 of the antisense strand from the 5′-end. Moreover, alternative 2′-OMe and 2′-F moieties are employed at other positions in both strands. In some embodiments, 2′-OMe and 2′-F are complementarily used for all positions of both strands of siRNA.

In some embodiments, a siRNA described herein comprises modifications to the overhangs and/or termini. In some cases, the 3′ end overhang is modified. In some cases, the 5′ end overhang is modified. In some cases, the 5′ blunt terminus and/or the 3′ end blunt terminus are modified. In other cases, the 3′ and 5′ end overhangs are modified.

The termini of siRNA can be modified in a variety of ways, for example, phosphorylation at the 5′ end of the sense strand. Cell penetrating peptides, steroids, lipids, and other moieties can be linked to the siRNA termini to enhance the delivery of siRNA.

In some embodiments, a siRNA described herein may comprise modifications to the duplex (i.e., the doubled-stranded) region.

siRNA Conjugates

Diverse ligands including small molecules, carbohydrates, lipids, aptamers, peptides and antibodies can be covalently linked to siRNA in order to improve cellular uptake, target specific cell types, increase stability of the siRNAs and reduce the immunogenicity of the siRNAs, etc. An advantage of siRNA conjugates is that they reduce the need for extra delivery materials, and may thereby improve the tolerability and safety profile of the delivery formulation.

For example, the conjugation of a synthetic tri-antennary N-acetylgalactosamine (GalNAc) ligand to a siRNA molecule can increase delivery of siRNA therapeutics to hepatocytes in vivo as the Tri-GalNAc ligand can bind with high affinity and specificity to the asialoglycoprotein receptor (ASGPR), a hepatocyte transmembrane glycoprotein that is highly expressed on hepatocytes. GalNAc-siRNA conjugates have sustained effect on silencing the expression of the target gene.

In some embodiments, the siRNA is conjugated to one or more N-acetyl-D-galactose (GalNAc) residues. A siRNA that is conjugated to N-acetyl-D-galactose is referred to herein as a “GalNAc siRNA conjugate.” The following patent documents describe methods for conjugating nucleic acids like siRNAs to GalNAc: U.S. Pat. No. 8,575,123; U.S. Publication No. 2009/0239814; and U.S. Pat. No. 9,708,607. Each of the aforementioned patent documents is incorporated by reference herein in its entirety. In some cases, the GalNAc may have any structure described in U.S. Pat. Nos. 8,575,123; 8,106,022; 8,828,956; 9,506,306; 8,313,772; 9,011,919; 9,345,775; and 10,316,316; and U.S. Publication No. 2009/0239814; and U.S. Pat. No. 9,708,607, except that the nucleic acid sequence described in these patent documents is replaced by a siRNA described herein. In other cases, the GAlNAc is a derivative disclosed in US Application Publication No. US20200270611, the contents of which are incorporated herein by reference in their entirety. Without being bound by theory, GalNAc siRNA conjugates bind the asialoglycoprotein receptor (ASGPR). ASGPR is selectively expressed in hepatocytes. The interaction between GAlNAc and ASGPR leads to uptake of a GalNAc siRNA conjugate to a specific cell (e.g., hepatocyte).

Conjugation can be performed on the sense strand, e.g., 5′ end and/or 3′ end of the sense strand of the siRNA. Conjugation can also be performed on the antisense strand, e.g., 3′ end of the antisense strand. In some cases, conjugation can also be performed at the 5′ end of the antisense strand though the 5′ end of the antisense strand is required for silencing activity of the siRNA.

In some embodiments, a conjugate is linked to a nucleotide of a siRNA directly, for example, via a phosphodiester bond. In some embodiments, a linker is used for conjugating a siRNA to one or more conjugates (e.g., GalNAc). The conjugated siRNA may have a formula of A-L-B (A=siRNA; L=linker and B=conjugate). In some embodiments, the linker is a bivalent C₁-C₅₀ saturated or unsaturated, straight or branched alkyl, wherein 1-25 methylene groups are optionally and independently replaced by —N(H)—, —N(C₁-C₄ alkyl)-, —N(cycloalkyl)-, —O—, —C(O)—, —C(O)O—, —S—, —S(O)—, —S(O)₂—, —S(O)₂N(C₁-C₄ alkyl)-, —S(O)₂N(cycloalkyl)-, —N(H)C(O)—, —N(C₁-C₄ alkyl)C(O)—, —N(cycloalkyl)C(O)—, —C(O)N(H)—, —C(O)N(C₁-C₄ alkyl), —C(O)N(cycloalkyl), aryl, heteroaryl, cycloalkyl, or cycloalkenyl. In some embodiments, the linker is a non-cleavable linker. In other embodiments, the linker is a cleavable linker. The linker described herein can be a non-polymeric linker. A non-polymeric linker refers to a linker that does not contain a repeating unit of monomers generated by a polymerization process. Exemplary non-polymeric linkers include, but are not limited to, C₁-C₆ alkyl group (e.g., a C₅, C₄, C₃, C₂, or C₁ alkyl group), homobifunctional cross linkers, heterobifunctional cross linkers, peptide linkers, traceless linkers, self-immolative linkers, maleimide-based linkers, or combinations thereof. In some cases, the non-polymeric linker comprises a C₁-C₆ alkyl group (e.g., a C₅, C₄, C₃, C₂, or C₁ alkyl group), a homobifunctional cross linker, a heterobifunctional cross linker, a peptide linker, a traceless linker, a self-immolative linker, a maleimide-based linker, or a combination thereof. In additional cases, the non-polymeric linker does not comprise more than two of the same type of linkers, e.g., more than two homobifunctional cross linkers, or more than two peptide linkers. In further cases, the non-polymeric linker optionally comprises one or more reactive functional groups. In some embodiments, the linker is an aminohexyl linker (e.g., —(CH₂)₆NH—).

### Combinations

In some embodiments, a siRNA molecule may comprise a combination of various modifications and conjugates to achieve a desired effect, e.g., sustained silencing and in vivo stability. In some embodiments, combining 2′-O-methyl (2′-OMe) and 2′-deoxy-2′-fluoro (2′-F) ribosugar modifications throughout both strands of the siRNA with terminal phosphorothioate (PS) linkages can provide protection against 30 and 50 exonucleases.

In some embodiments, a siRNA molecule may comprise a combination of sugar, base and backbone modifications and a terminal GalNAc conjugate.

siRNA Synthesis

In some embodiments, siRNAs can be synthesized using solid-phase synthesis. RNA synthesis is a repetitive chemical cycle in which each nucleotide is added on a solid support. This cycle starts with a deprotection step to remove the protective group on 5′-hydroxyl of the solid support bound nucleotide. The resulting 5′-hydroxyl is then coupled with an activated 3′-phosphorous ester, followed by a capping step to remove the unreacted nucleotides from the reaction system. The intermediate undergoes another step to oxidize phosphite to phosphorous ester. After the chain assembly, the oligomer is released from the solid support, deprotected, and purified by HPLC.

In some embodiments, chemical modifications and conjugates and siRNA synthesis can be performed using parallel synthesis and linear synthesis. For parallel synthesis, siRNA and its relevant conjugate ligand are synthesized in separate synthetic routes and then are conjugated with each other usually through biodegradable bonds (e.g., Jeong et al., siRNA conjugate delivery systems. Bioconjugate Chem. 2009, 20, 5-14; the contents of which are incorporated herein by reference it their entirety.) Linear synthesis (functional groups are added sequentially) is also widely used for a variety of chemical conjugations to siRNA.

siRNA Delivery

A series of barriers need to be circumvented for systemically administered exogenous siRNA before it can achieve gene silencing. In blood circulation, unspecific binding and glomerular filtration both hamper the accumulation of siRNA in desired tissues. The neutral surface charge of siRNA-loaded nanoformulations is beneficial for avoiding unfavorable protein binding in circulation. siRNA is ˜7-8 nm in length and 2-3 nm in diameter. The relatively high molecular weight (˜13-16 kD) and net negative charge prevent artificial siRNA from crossing the cell membrane. Encapsulating siRNA into vesicles or conjugating it to certain ligands can effectively deliver siRNA to the desired tissues or cells.

### Lipid Based Delivery Systems

Phospholipids are natural components of cell membranes that form lipid bilayers. Liposomes comprising one or more synthetic lipids can be used for developing lipid-based siRNA delivery systems. In some embodiments, Lipid nanoparticles (LNPs) are used to deliver siRNA into cells. Lipid-based nanoparticles (LPNs), particularly lipids with single or multiple cationic centers are highly effective carriers of siRNA.

In some embodiments, Stabilized Nucleic Acid Lipid Particles (SNALPs) are used for intracellular delivery of siRNA molecules. SNALPs are typically composed of a formulation consisting of an amine-based lipid, cholesterol, a PEG-lipid, as well as helper phospholipids. For example, a LNP comprises DlinDMA, 1,2-distearoyl-sn-glycero-3-phosphocholine (DSPC) as a helper lipid, mPEGC-DMA, and cholesterol (DlinDMA:DSPC:Chol:PEG-C-DMA 30:20:48:2 molar percent (Heyes et al., Cationic lipid saturation influences intracellular delivery of encapsulated nucleic acids. J. Controlled Release 107, 2005, 276-287; the contents of which are incorporated herein by reference in their entireties.)

Cationic lipids (or amino lipids) are central components of LNPs, as they play a role in the assembly of the nanoparticles, by binding the siRNA through electrostatic interactions. These amino groups also facilitate endosomal escape, through interaction with endosomal components during acidification. A typical cationic lipid includes a cationic head group, a linker, and two long hydrophobic domains.

Exemplary cationic lipids include but are not limited to DLin-KC2-DMA, DLin-MC3-DMA, 7C1 and L319.

In addition to cation lipids with single cationic center, lipid derivatives and lipid-like materials with multiple cationic centers can also be used for developing lipid based siRNA delivery systems, for example, aminoglycoside and amino acid derivatives (e.g., an arginine based lipid, AtuFECT01). Aminoglycoside-based lipids are composed of an aminoglycosides head, an amide linker, and two unsaturated tails (e.g., DOPE).

Other lipid like molecules, e.g., lipidoids, can also be used for siRNA delivery. Lipidoids are composed of one or more amine centers and multiple hydrophobic tails. Diverse lipidoids can be synthesized with a range of functional amines and acrylates or acrylamides (for example through a one-step Michael addition reaction without the need of catalysts or solvents). The amino groups can function similarly to the amino head group in the lipid nanoparticles by neutralizing negative charges of siRNA and facilitating cytoplasmic release of siRNA.

Various lipidoids for siRNA delivery can be developed using different methods such as epoxide-based chemistry.

Exemplary lipidoids include C12-200, cKK-E12. C12-200 is composed of a piperazine ring and five lipid tails. The ED₅₀ of siRNA formulated with C12-200 was reported as low as 0.01 mg/kg for hepatocytes in mice. The potency of this formulation also allows the potential for simultaneous gene silencing. C12-200 shows to be the most potent and selective siRNA delivery systems for gene silencing reported thus far for hepatocytes (Dong, et al. Lipopeptide nanoparticles for potent and selective siRNA delivery in rodents and nonhuman primates. Proc Natl Acad Sci USA, 2014, 111(11):3955-3960).

Polymer-Based siRNA Delivery Systems

One or more polymers may be used for siRNA delivery.

Interaction between positively charged groups on polymer chains and the negatively charged phosphates in siRNA molecules can allow polymeric carriers to complex siRNA.

In some embodiments, siRNAs to be delivered can be directly conjugated to polymers to improve cellular uptake, and delivery of the siRNAs. Polymers such as poly(ethylene glycol) (PEG) and poly(lactic-co-glycolic acid) (PLGA) have been conjugated to siRNA to facilitate delivery.

In some cases, block copolymers can be used for siRNA delivery. For example, PLGA-based block copolymer systems are used for systemic delivery of siRNA against various targets.

The use of well-defined polymer architecture and precise cross-linking chemistry are also useful strategies for siRNA delivery. Incorporation of two or three cysteine cross-links into poly(amido amines) is critical to achieve siRNA delivery (Schaffert, et al. Solid-phase synthesis of sequence-defined T-, i-, and U-shape polymers for pDNA and siRNA delivery. Angew. Chem. Int. Ed 2011, 50, 8986-8989).

In some cases, natural biopolymers can be used for siRNA delivery. It has been shown that cyclodextrin-based self-assembling polymeric nanoparticles can facilitate siRNA delivery.

Additionally, proteins can be used for siRNA delivery. A protamine-HIV-1 envelope antibody fusion protein can deliver siRNA selectively to cells expressing the HIV-1 envelope (Song et al., Antibody mediated in vivo delivery of small interfering RNAs via cell-surface receptors. Nat. Biotechnol, 2005, 23, 709-717).

Exemplary siRNA Molecules Targeting a SLC10A1 mRNA Transcript

The present disclosure, by bioinformatics-based selection and knockdown of the expression of SLC10A1 mRNA, identified siRNA molecules that can repress the expression of a SLC10A1 mRNA transcript specifically and effectively. In accordance, the present disclosure provides siRNA molecules with specificity and high efficacy to repress the expression of a human SLC10A1 mRNA transcript in a cell, e.g., a hepatocyte. In some embodiments, the siRNA molecule targeting the human SLC10A1 mRNA transcript comprises a sense strand comprising a sequence selected from the group consisting of SEQ ID NOs. 1-15 and 481-611, and an antisense strand comprising a sequence complementary to the sense strand sequence.

In some embodiments, the siRNA molecule described herein comprises an antisense strand comprising a sequence selected from the group consisting of SEQ ID NOs. 16-30 and 612-742, and a sense strand comprising a sequence complementary of the antisense strand sequence.

In some cases, the siRNA molecule described herein comprises a sense strand comprises a sequence at least about 80%, 85%, 90%, 95%, 96%, 97%, 98%, 99%, or 100% identical to a sequence selected from the group consisting of SEQ ID NOs. 1-15 and 481-611, and an antisense strand comprises a sequence at least about 80%, 85%, 90%, 95%, 96%, 97%, 98%, 99%, or 100% identical to the reverse complement of the sense strand of any one of SEQ ID NOs. 1-15 and 481-611. In some cases, the siRNA molecule described herein comprises an antisense strand comprises a sequence at least about 80%, 85%, 90%, 95%, 96%, 97%, 98%, 99%, or 100% identical to a sequence selected from SEQ ID NOs. 16-30 and 612-742 and a sense strand comprises a sequence at least about 80%, 85%, 90%, 95%, 96%, 97%, 98%, 99%, or 100% identical to the reverse complement of the antisense strand of any one of SEQ ID NOs. 16-30 and 612-742.

In some embodiments, the siRNA molecule described herein comprises a sense strand having a sequence of SEQ ID NO. 1 and an antisense strand having a sequence of SEQ ID NO. 16. In some embodiments, the siRNA molecule described herein comprises a sense strand having a sequence of SEQ ID NO. 2 and an antisense strand having a sequence of SEQ ID NO. 17. In some embodiments, the siRNA molecule described herein comprises a sense strand having a sequence of SEQ ID NO. 3 and an antisense strand having a sequence of SEQ ID NO. 18. In some embodiments, the siRNA molecule described herein comprises a sense strand having a sequence of SEQ ID NO. 4 and an antisense strand having a sequence of SEQ ID NO. 19. In some embodiments, the siRNA molecule described herein comprises a sense strand having a sequence of SEQ ID NO. 5 and an antisense strand having a sequence of SEQ ID NO. 20. In some embodiments, the siRNA molecule described herein comprises a sense strand having a sequence of SEQ ID NO. 6 and an antisense strand having a sequence of SEQ ID NO. 21. In some embodiments, the siRNA molecule described herein comprises a sense strand having a sequence of SEQ ID NO. 7 and an antisense strand having a sequence of SEQ ID NO. 22. In some embodiments, the siRNA molecule described herein comprises a sense strand having a sequence of SEQ ID NO. 8 and an antisense strand having a sequence of SEQ ID NO. 23. In some embodiments, the siRNA molecule described herein comprises a sense strand having a sequence of SEQ ID NO. 9 and an antisense strand having a sequence of SEQ ID NO. 24. In some embodiments, the siRNA molecule described herein comprises a sense strand having a sequence of SEQ ID NO. 10 and an antisense strand having a sequence of SEQ ID NO. 25. In some embodiments, the siRNA molecule described herein comprises a sense strand having a sequence of SEQ ID NO. 11 and an antisense strand having a sequence of SEQ ID NO. 26. In some embodiments, the siRNA molecule described herein comprises a sense strand having a sequence of SEQ ID NO. 12 and an antisense strand having a sequence of SEQ ID NO. 27. In some embodiments, the siRNA molecule described herein comprises a sense strand having a sequence of SEQ ID NO. 13 and an antisense strand having a sequence of SEQ ID NO. 28. In some embodiments, the siRNA molecule described herein comprises a sense strand having a sequence of SEQ ID NO. 14 and an antisense strand having a sequence of SEQ ID NO. 29. In some embodiments, the siRNA molecule described herein comprises a sense strand having a sequence of SEQ ID NO. 15 and an antisense strand having a sequence of SEQ ID NO. 30.

TABLE 1
Sense and antisense sequences of siRNA molecules

| | Sense strand | Antisense strand |
|---|---|---|
| Duplex ID | sequence (5′-3′) | sequence (5′-3′) |
| SR612 | AGAUCAAGGCUCACUUAUG | CAUAAGUGAGCCUUGAUCUUG |
| | (SEQ ID NO. 1) | (SEQ ID NO. 16) |
| SR670 | UCAGCAUUGUGAUGACCAC | GUGGUCAUCACAAUGCUGAGG |
| | (SEQ ID NO. 2) | (SEQ ID NO. 17) |
| SR710 | UCAUCAUUCUCUUGUGCAG | CUGCACAAGAGAAUGAUGAUC |
| | (SEQ ID NO. 3) | (SEQ ID NO. 18) |
| SR412 | UGGCCGUCACAGUUCUCUC | GAGAGAACUGUGACGGCCACA |
| | (SEQ ID NO. 4) | (SEQ ID NO. 19) |
| SR508 | UGGUCCCAAAGCAAUUCUG | CAGAAUUGCUUUGGGACCAGA |
| | (SEQ ID NO. 5) | (SEQ ID NO. 20) |
| SR455 | UCCAGUACAUCUAUCAGAA | UUCUGAUAGAUGUACUGGAAA |
| | (SEQ ID NO. 6) | (SEQ ID NO. 21) |
| SR530 | CCAGUACAUCUAUCAGAAU | AUUCUGAUAGAUGUACUGGAA |
| | (SEQ ID NO. 7) | (SEQ ID NO. 22) |
| SR378 | AUCUAUCAGAAUCAUCAAG | CUUGAUGAUUCUGAUAGAUGU |
| | (SEQ ID NO. 8) | (SEQ ID NO. 23) |
| SR579 | AAGUCACUCAAUUCCUGAA | UUCAGGAAUUGAGUGACUUUA |
| | (SEQ ID NO. 9) | (SEQ ID NO. 24) |
| SR641 | CCUGAACUAAUCAAUAUAU | AUAUAUUGAUUAGUUCAGGAA |
| | (SEQ ID NO. 10) | (SEQ ID NO. 25) |
| SR417 | UGAACUAAUCAAUAUAUAU | AUAUAUAUUGAUUAGUUCAGG |
| | (SEQ ID NO. 11) | (SEQ ID NO. 26) |
| SR512 | UGGUUCUCAUUCCUUGCAC | GUGCAAGGAAUGAGAACCAGG |
| | (SEQ ID NO. 12) | (SEQ ID NO. 27) |
| SR497 | CUCAUUCCUUGCACCAUAG | CUAUGGUGCAAGGAAUGAGAA |
| | (SEQ ID NO. 13) | (SEQ ID NO. 28) |
| SR572 | UUGGCUUUCUGCUGGGUUA | UAACCCAGCAGAAAGCCAAUA |
| | (SEQ ID NO. 14) | (SEQ ID NO. 29) |
| SR677 | UCCUCUACAUGAUUUUCCA | UGGAAAAUCAUGUAGAGGAGG |
| | (SEQ ID NO. 15) | (SEQ ID NO. 30) |

In some embodiments, the siRNA molecule specific to SLC10A1 comprises at least one overhang at one end.

In some embodiments, the siRNA molecule specific to SLC10A1 comprises a sense strand having 19 nucleotides in length and an antisense strand having 21 nucleotide in length. The sense and antisense strands form a duplex of 19 base pairs.

In some embodiments, the last nucleotide of the sense strand of a siRNA molecule is modified as adenine (A). In some embodiments, the first nucleotide of the antisense strand of a siRNA molecule is modified as uracil (U).

As non-limiting examples, the siRNA molecule described herein comprises a sense strand having a sequence of SEQ ID NO. 31 and an antisense strand having a sequence of SEQ ID NO. 46. In some embodiments, the siRNA molecule described herein comprises a sense strand having a sequence of SEQ ID NO. 32 and an antisense strand having a sequence of SEQ ID NO. 47. In some embodiments, the siRNA molecule described herein comprises a sense strand having a sequence of SEQ ID NO. 33 and an antisense strand having a sequence of SEQ ID NO. 48. In some embodiments, the siRNA molecule described herein comprises a sense strand having a sequence of SEQ ID NO. 34 and an antisense strand having a sequence of SEQ ID NO. 49. In some embodiments, the siRNA molecule described herein comprises a sense strand having a sequence of SEQ ID NO. 35 and an antisense strand having a sequence of SEQ ID NO. 50. In some embodiments, the siRNA molecule described herein comprises a sense strand having a sequence of SEQ ID NO. 36 and an antisense strand having a sequence of SEQ ID NO. 51. In some embodiments, the siRNA molecule described herein comprises a sense strand having a sequence of SEQ ID NO. 37 and an antisense strand having a sequence of SEQ ID NO. 52. In some embodiments, the siRNA molecule described herein comprises a sense strand having a sequence of SEQ ID NO. 38 and an antisense strand having a sequence of SEQ ID NO. 53. In some embodiments, the siRNA molecule described herein comprises a sense strand having a sequence of SEQ ID NO. 39 and an antisense strand having a sequence of SEQ ID NO. 54. In some embodiments, the siRNA molecule described herein comprises a sense strand having a sequence of SEQ ID NO. 40 and an antisense strand having a sequence of SEQ ID NO. 55. In some embodiments, the siRNA molecule described herein comprises a sense strand having a sequence of SEQ ID NO. 41 and an antisense strand having a sequence of SEQ ID NO. 56. In some embodiments, the siRNA molecule described herein comprises a sense strand having a sequence of SEQ ID NO. 42 and an antisense strand having a sequence of SEQ ID NO. 57. In some embodiments, the siRNA molecule described herein comprises a sense strand having a sequence of SEQ ID NO. 43 and an antisense strand having a sequence of SEQ ID NO. 58. In some embodiments, the siRNA molecule described herein comprises a sense strand having a sequence of SEQ ID NO. 44 and an antisense strand having a sequence of SEQ ID NO. 59. In some embodiments, the siRNA molecule described herein comprises a sense strand having a sequence of SEQ ID NO. 45 and an antisense strand having a sequence of SEQ ID NO. 60.

TABLE 2
Modified sense strand sequences and
antisense strand sequences of siRNAs

| | Sense strand | Antisense strand |
|---|---|---|
| | sequence (5′-3′) | sequence (5′-3′) |
| ID | (Position 19 = A) | (Position 1 = U) |
| SR387 | AGAUCAAGGCUCACUUAUA | UAUAAGUGAGCCUUGAUCUUG |
| | (SEQ ID NO. 31) | (SEQ ID NO. 46) |
| SR401 | UCAGCAUUGUGAUGACCAA | UUGGUCAUCACAAUGCUGAGG |
| | (SEQ ID NO. 32) | (SEQ ID NO. 47) |
| SR461 | UCAUCAUUCUCUUGUGCAA | UUGCACAAGAGAAUGAUGAUC |
| | (SEQ ID NO. 33) | (SEQ ID NO. 48) |
| SR413 | UGGCCGUCACAGUUCUCUA | UAGAGAACUGUGACGGCCACA |
| | (SEQ ID NO. 34) | (SEQ ID NO. 49) |
| SR509 | UGGUCCCAAAGCAAUUCUA | UAGAAUUGCUUUGGGACCAGA |
| | (SEQ ID NO. 35) | (SEQ ID NO. 50) |
| SR455 | UCCAGUACAUCUAUCAGAA | UUCUGAUAGAUGUACUGGAAA |
| | (SEQ ID NO. 36) | (SEQ ID NO. 51) |
| SR573 | CCAGUACAUCUAUCAGAAA | UUUCUGAUAGAUGUACUGGAA |
| | (SEQ ID NO. 37) | (SEQ ID NO. 52) |
| SR689 | AUCUAUCAGAAUCAUCAAA | UUUGAUGAUUCUGAUAGAUGU |
| | (SEQ ID NO. 38) | (SEQ ID NO. 53) |
| SR579 | AAGUCACUCAAUUCCUGAA | UUCAGGAAUUGAGUGACUUUA |
| | (SEQ ID NO. 39) | (SEQ ID NO. 54) |
| SR541 | CCUGAACUAAUCAAUAUAA | UUAUAUUGAUUAGUUCAGGAA |
| | (SEQ ID NO. 40) | (SEQ ID NO. 55) |
| SR403 | UGAACUAAUCAAUAUAUAA | UUAUAUAUUGAUUAGUUCAGG |
| | (SEQ ID NO. 41) | (SEQ ID NO. 56) |
| SR555 | UGGUUCUCAUUCCUUGCAA | UUGCAAGGAAUGAGAACCAGG |
| | (SEQ ID NO. 42) | (SEQ ID NO. 57) |
| SR457 | CUCAUUCCUUGCACCAUAA | UUAUGGUGCAAGGAAUGAGAA |
| | (SEQ ID NO. 43) | (SEQ ID NO. 58) |
| SR572 | UUGGCUUUCUGCUGGGUUA | UAACCCAGCAGAAAGCCAAUA |
| | (SEQ ID NO. 44) | (SEQ ID NO. 59) |
| SR677 | UCCUCUACAUGAUUUUCCA | UGGAAAAUCAUGUAGAGGAGG |
| | (SEQ ID NO. 45) | (SEQ ID NO. 60) |

In some embodiments, the siRNA molecule specific to SLC10A1 comprises a sense strand having 21 nucleotides in length and an antisense strand having 23 nucleotide in length. The sense and antisense strands form a duplex of 21 base pairs.

Nucleotides of a nucleic acid sequence can be chemically modified. In some embodiments of the present disclosure, a siRNA targeting a SLC10A1 mRNA transcript comprises at least one chemical modification. In some embodiments, the sense strand of a siRNA molecule described herein comprises at least one modification. In some embodiments, the antisense strand of a siRNA molecule described herein comprises at least one modification. In some embodiments, the sense strand and the antisense strand of a siRNA molecule described herein comprise at least one modification.

In some embodiments, the sense strand comprises 1, 2, 3, 4, 5, 6, 7, 8, 9, 10, 11, 12, 13, 14, 15, 16, 17, 18, 19, 20, or 21 modified nucleosides. In some embodiments, the antisense strand comprises 1, 2, 3, 4, 5, 6, 7, 8, 9, 10, 11, 12, 13, 14, 15, 16, 17, 18, 19, 20, 21 or 23 modified nucleosides. In some embodiments, the siRNA molecule described herein comprises a range of nucleosides defined by any two of the aforementioned numbers. In some embodiments, the siRNA molecule described herein comprises no more than 19 modified nucleosides. In some embodiments, the siRNA molecule described herein comprises no more than 21 modified nucleosides. In some embodiments, the siRNA molecule comprises 2 or more modified nucleosides, 3 or more modified nucleosides, 4 or more modified nucleosides, 5 or more modified nucleosides, 6 or more modified nucleosides, 7 or more modified nucleosides, 8 or more modified nucleosides, 9 or more modified nucleosides, 10 or more modified nucleosides, 11 or more modified nucleosides, 12 or more modified nucleosides, 13 or more modified nucleosides, 14 or more modified nucleosides, 15 or more modified nucleosides, 16 or more modified nucleosides, 17 or more modified nucleosides, 18 or more modified nucleosides, 19 or more modified nucleosides, 20 or more modified nucleosides, 21 or more modified nucleosides, 22 or more modified nucleosides, or 23 or more modified nucleosides.

Chemically modified nucleotides comprise but are not limited to 2′-O-methyl, 2′-methoxyethyl, 2′-O-alkyl, 2′-O-allyl, 2′-O-allyl, 2′-fluoro, or 2′-deoxy modified nucleotides, or a combination thereof. In some embodiments, the modified nucleotide comprises a 2′-O-methyl nucleotide, 2′-deoxyfluoro nucleotide, 2′-O—N-methylacetamido (2′-O-NMA) nucleotide, a 2′-O-dimethylaminocthoxyethyl (2′-O-DMAEOE) nucleotide, 2′-O-aminopropyl (2′-O-AP) nucleotide, 2′-fluoro nucleotide, or 2′-ara-F, or a combination thereof. In some embodiments, the modified nucleotide comprises one or more 2′-O-methyl modified nucleotides. In some embodiments, the modified nucleotide comprises one or more 2′-fluoro modified nucleotides. In some embodiments, the modified nucleotides comprise one or more 2′-O-methyl modified nucleotides and one or more 2′-fluoro modified nucleotides.

In some embodiments, one or more nucleotides in the sense and/or antisense strand of a siRNA molecule are modified. In some cases, every nucleotide in the sense strand and antisense strand of the siRNA molecule is modified. The modifications on sense strand and antisense strand may each independently comprise at least two different modifications. In some embodiments, not every nucleotide in the sense and antisense strand is modified. In some embodiments, no nucleotide in the sense and/or antisense strand is modified. In some embodiments, the sense strand of the siRNA molecule comprises a modification pattern as described herein. In some embodiments, the antisense strand of the siRNA molecule comprises a modification pattern as described herein. In some embodiments, the sense and antisense strands of the siRNA molecule comprise modification patterns as described herein.

In some embodiments, the sense strand of the siRNA molecule comprises a modification pattern in Table 3. In another example, the antisense strand of the siRNA molecule comprises a modification pattern in Table 3. In some embodiments, the siRNA molecule comprises a modified sense strand sequence of any one of SEQ ID NOs. 61-270. In some embodiments, the siRNA molecule comprises a modified antisense strand sequence of any one of SEQ ID NOs. 271-480.

In some embodiments, the siRNA molecule comprises a modified sense strand sequence of SEQ ID NO. 61 and a modified antisense strand sequence of SEQ ID NO. 271. In some embodiments, the siRNA molecule comprises a modified sense strand sequence of SEQ ID NO. 62 and a modified antisense strand sequence of SEQ ID NO. 272. In some embodiments, the siRNA molecule comprises a modified sense strand sequence of SEQ ID NO. 63 and a modified antisense strand sequence of SEQ ID NO. 273. In some embodiments, the siRNA molecule comprises a modified sense strand sequence of SEQ ID NO. 64 and a modified antisense strand sequence of SEQ ID NO. 274. In some embodiments, the siRNA molecule comprises a modified sense strand sequence of SEQ ID NO. 65 and a modified antisense strand sequence of SEQ ID NO. 275. In some embodiments, the siRNA molecule comprises a modified sense strand sequence of SEQ ID NO. 66 and a modified antisense strand sequence of SEQ ID NO. 276. In some embodiments, the siRNA molecule comprises a modified sense strand sequence of SEQ ID NO. 67 and a modified antisense strand sequence of SEQ ID NO. 277. In some embodiments, the siRNA molecule comprises a modified sense strand sequence of SEQ ID NO. 68 and a modified antisense strand sequence of SEQ ID NO. 278. In some embodiments, the siRNA molecule comprises a modified sense strand sequence of SEQ ID NO. 69 and a modified antisense strand sequence of SEQ ID NO. 279. In some embodiments, the siRNA molecule comprises a modified sense strand sequence of SEQ ID NO. 70 and a modified antisense strand sequence of SEQ ID NO. 280. In some embodiments, the siRNA molecule comprises a modified sense strand sequence of SEQ ID NO. 71 and a modified antisense strand sequence of SEQ ID NO. 281. In some embodiments, the siRNA molecule comprises a modified sense strand sequence of SEQ ID NO. 72 and a modified antisense strand sequence of SEQ ID NO. 282. In some embodiments, the siRNA molecule comprises a modified sense strand sequence of SEQ ID NO. 73 and a modified antisense strand sequence of SEQ ID NO. 283. In some embodiments, the siRNA molecule comprises a modified sense strand sequence of SEQ ID NO. 74 and a modified antisense strand sequence of SEQ ID NO. 284. In some embodiments, the siRNA molecule comprises a modified sense strand sequence of SEQ ID NO. 75 and a modified antisense strand sequence of SEQ ID NO. 285. In some embodiments, the siRNA molecule comprises a modified sense strand sequence of SEQ ID NO. 76 and a modified antisense strand sequence of SEQ ID NO. 286. In some embodiments, the siRNA molecule comprises a modified sense strand sequence of SEQ ID NO. 77 and a modified antisense strand sequence of SEQ ID NO. 287. In some embodiments, the siRNA molecule comprises a modified sense strand sequence of SEQ ID NO. 78 and a modified antisense strand sequence of SEQ ID NO. 288. In some embodiments, the siRNA molecule comprises a modified sense strand sequence of SEQ ID NO. 79 and a modified antisense strand sequence of SEQ ID NO. 289. In some embodiments, the siRNA molecule comprises a modified sense strand sequence of SEQ ID NO. 80 and a modified antisense strand sequence of SEQ ID NO. 290. In some embodiments, the siRNA molecule comprises a modified sense strand sequence of SEQ ID NO. 81 and a modified antisense strand sequence of SEQ ID NO. 291. In some embodiments, the siRNA molecule comprises a modified sense strand sequence of SEQ ID NO. 82 and a modified antisense strand sequence of SEQ ID NO. 292. In some embodiments, the siRNA molecule comprises a modified sense strand sequence of SEQ ID NO. 83 and a modified antisense strand sequence of SEQ ID NO. 293. In some embodiments, the siRNA molecule comprises a modified sense strand sequence of SEQ ID NO. 84 and a modified antisense strand sequence of SEQ ID NO. 294. In some embodiments, the siRNA molecule comprises a modified sense strand sequence of SEQ ID NO. 85 and a modified antisense strand sequence of SEQ ID NO. 295. In some embodiments, the siRNA molecule comprises a modified sense strand sequence of SEQ ID NO. 86 and a modified antisense strand sequence of SEQ ID NO. 296. In some embodiments, the siRNA molecule comprises a modified sense strand sequence of SEQ ID NO. 87 and a modified antisense strand sequence of SEQ ID NO. 297. In some embodiments, the siRNA molecule comprises a modified sense strand sequence of SEQ ID NO. 88 and a modified antisense strand sequence of SEQ ID NO. 298. In some embodiments, the siRNA molecule comprises a modified sense strand sequence of SEQ ID NO. 89 and a modified antisense strand sequence of SEQ ID NO. 299. In some embodiments, the siRNA molecule comprises a modified sense strand sequence of SEQ ID NO. 90 and a modified antisense strand sequence of SEQ ID NO. 300. In some embodiments, the siRNA molecule comprises a modified sense strand sequence of SEQ ID NO. 91 and a modified antisense strand sequence of SEQ ID NO. 301. In some embodiments, the siRNA molecule comprises a modified sense strand sequence of SEQ ID NO. 92 and a modified antisense strand sequence of SEQ ID NO. 302. In some embodiments, the siRNA molecule comprises a modified sense strand sequence of SEQ ID NO. 93 and a modified antisense strand sequence of SEQ ID NO. 303. In some embodiments, the siRNA molecule comprises a modified sense strand sequence of SEQ ID NO. 94 and a modified antisense strand sequence of SEQ ID NO. 304. In some embodiments, the siRNA molecule comprises a modified sense strand sequence of SEQ ID NO. 95 and a modified antisense strand sequence of SEQ ID NO. 305. In some embodiments, the siRNA molecule comprises a modified sense strand sequence of SEQ ID NO. 96 and a modified antisense strand sequence of SEQ ID NO. 306. In some embodiments, the siRNA molecule comprises a modified sense strand sequence of SEQ ID NO. 97 and a modified antisense strand sequence of SEQ ID NO. 307. In some embodiments, the siRNA molecule comprises a modified sense strand sequence of SEQ ID NO. 98 and a modified antisense strand sequence of SEQ ID NO. 308. In some embodiments, the siRNA molecule comprises a modified sense strand sequence of SEQ ID NO. 99 and a modified antisense strand sequence of SEQ ID NO. 309. In some embodiments, the siRNA molecule comprises a modified sense strand sequence of SEQ ID NO. 100 and a modified antisense strand sequence of SEQ ID NO. 310. In some embodiments, the siRNA molecule comprises a modified sense strand sequence of SEQ ID NO. 101 and a modified antisense strand sequence of SEQ ID NO. 311. In some embodiments, the siRNA molecule comprises a modified sense strand sequence of SEQ ID NO. 102 and a modified antisense strand sequence of SEQ ID NO. 312. In some embodiments, the siRNA molecule comprises a modified sense strand sequence of SEQ ID NO. 103 and a modified antisense strand sequence of SEQ ID NO. 313. In some embodiments, the siRNA molecule comprises a modified sense strand sequence of SEQ ID NO. 104 and a modified antisense strand sequence of SEQ ID NO. 314. In some embodiments, the siRNA molecule comprises a modified sense strand sequence of SEQ ID NO. 105 and a modified antisense strand sequence of SEQ ID NO. 315. In some embodiments, the siRNA molecule comprises a modified sense strand sequence of SEQ ID NO. 106 and a modified antisense strand sequence of SEQ ID NO. 316. In some embodiments, the siRNA molecule comprises a modified sense strand sequence of SEQ ID NO. 107 and a modified antisense strand sequence of SEQ ID NO. 317. In some embodiments, the siRNA molecule comprises a modified sense strand sequence of SEQ ID NO. 108 and a modified antisense strand sequence of SEQ ID NO. 318. In some embodiments, the siRNA molecule comprises a modified sense strand sequence of SEQ ID NO. 109 and a modified antisense strand sequence of SEQ ID NO. 319. In some embodiments, the siRNA molecule comprises a modified sense strand sequence of SEQ ID NO. 110 and a modified antisense strand sequence of SEQ ID NO. 320. In some embodiments, the siRNA molecule comprises a modified sense strand sequence of SEQ ID NO. 111 and a modified antisense strand sequence of SEQ ID NO. 321. In some embodiments, the siRNA molecule comprises a modified sense strand sequence of SEQ ID NO. 112 and a modified antisense strand sequence of SEQ ID NO. 322. In some embodiments, the siRNA molecule comprises a modified sense strand sequence of SEQ ID NO. 113 and a modified antisense strand sequence of SEQ ID NO. 323. In some embodiments, the siRNA molecule comprises a modified sense strand sequence of SEQ ID NO. 114 and a modified antisense strand sequence of SEQ ID NO. 324. In some embodiments, the siRNA molecule comprises a modified sense strand sequence of SEQ ID NO. 115 and a modified antisense strand sequence of SEQ ID NO. 325. In some embodiments, the siRNA molecule comprises a modified sense strand sequence of SEQ ID NO. 116 and a modified antisense strand sequence of SEQ ID NO. 326. In some embodiments, the siRNA molecule comprises a modified sense strand sequence of SEQ ID NO. 117 and a modified antisense strand sequence of SEQ ID NO. 327. In some embodiments, the siRNA molecule comprises a modified sense strand sequence of SEQ ID NO. 118 and a modified antisense strand sequence of SEQ ID NO. 328. In some embodiments, the siRNA molecule comprises a modified sense strand sequence of SEQ ID NO. 119 and a modified antisense strand sequence of SEQ ID NO. 329. In some embodiments, the siRNA molecule comprises a modified sense strand sequence of SEQ ID NO. 120 and a modified antisense strand sequence of SEQ ID NO. 330. In some embodiments, the siRNA molecule comprises a modified sense strand sequence of SEQ ID NO. 121 and a modified antisense strand sequence of SEQ ID NO. 331. In some embodiments, the siRNA molecule comprises a modified sense strand sequence of SEQ ID NO. 122 and a modified antisense strand sequence of SEQ ID NO. 332. In some embodiments, the siRNA molecule comprises a modified sense strand sequence of SEQ ID NO. 123 and a modified antisense strand sequence of SEQ ID NO. 333. In some embodiments, the siRNA molecule comprises a modified sense strand sequence of SEQ ID NO. 124 and a modified antisense strand sequence of SEQ ID NO. 334. In some embodiments, the siRNA molecule comprises a modified sense strand sequence of SEQ ID NO. 125 and a modified antisense strand sequence of SEQ ID NO. 335. In some embodiments, the siRNA molecule comprises a modified sense strand sequence of SEQ ID NO. 126 and a modified antisense strand sequence of SEQ ID NO. 336. In some embodiments, the siRNA molecule comprises a modified sense strand sequence of SEQ ID NO. 127 and a modified antisense strand sequence of SEQ ID NO. 337. In some embodiments, the siRNA molecule comprises a modified sense strand sequence of SEQ ID NO. 128 and a modified antisense strand sequence of SEQ ID NO. 338. In some embodiments, the siRNA molecule comprises a modified sense strand sequence of SEQ ID NO. 129 and a modified antisense strand sequence of SEQ ID NO. 339. In some embodiments, the siRNA molecule comprises a modified sense strand sequence of SEQ ID NO. 130 and a modified antisense strand sequence of SEQ ID NO. 340. In some embodiments, the siRNA molecule comprises a modified sense strand sequence of SEQ ID NO. 131 and a modified antisense strand sequence of SEQ ID NO. 341. In some embodiments, the siRNA molecule comprises a modified sense strand sequence of SEQ ID NO. 132 and a modified antisense strand sequence of SEQ ID NO. 342. In some embodiments, the siRNA molecule comprises a modified sense strand sequence of SEQ ID NO. 133 and a modified antisense strand sequence of SEQ ID NO. 343. In some embodiments, the siRNA molecule comprises a modified sense strand sequence of SEQ ID NO. 134 and a modified antisense strand sequence of SEQ ID NO. 344. In some embodiments, the siRNA molecule comprises a modified sense strand sequence of SEQ ID NO. 135 and a modified antisense strand sequence of SEQ ID NO. 345. In some embodiments, the siRNA molecule comprises a modified sense strand sequence of SEQ ID NO. 136 and a modified antisense strand sequence of SEQ ID NO. 346. In some embodiments, the siRNA molecule comprises a modified sense strand sequence of SEQ ID NO. 137 and a modified antisense strand sequence of SEQ ID NO. 347. In some embodiments, the siRNA molecule comprises a modified sense strand sequence of SEQ ID NO. 138 and a modified antisense strand sequence of SEQ ID NO. 348. In some embodiments, the siRNA molecule comprises a modified sense strand sequence of SEQ ID NO. 139 and a modified antisense strand sequence of SEQ ID NO. 349. In some embodiments, the siRNA molecule comprises a modified sense strand sequence of SEQ ID NO. 140 and a modified antisense strand sequence of SEQ ID NO. 350. In some embodiments, the siRNA molecule comprises a modified sense strand sequence of SEQ ID NO. 141 and a modified antisense strand sequence of SEQ ID NO. 351. In some embodiments, the siRNA molecule comprises a modified sense strand sequence of SEQ ID NO. 142 and a modified antisense strand sequence of SEQ ID NO. 352. In some embodiments, the siRNA molecule comprises a modified sense strand sequence of SEQ ID NO. 143 and a modified antisense strand sequence of SEQ ID NO. 353. In some embodiments, the siRNA molecule comprises a modified sense strand sequence of SEQ ID NO. 144 and a modified antisense strand sequence of SEQ ID NO. 354. In some embodiments, the siRNA molecule comprises a modified sense strand sequence of SEQ ID NO. 145 and a modified antisense strand sequence of SEQ ID NO. 355. In some embodiments, the siRNA molecule comprises a modified sense strand sequence of SEQ ID NO. 146 and a modified antisense strand sequence of SEQ ID NO. 356. In some embodiments, the siRNA molecule comprises a modified sense strand sequence of SEQ ID NO. 147 and a modified antisense strand sequence of SEQ ID NO. 357. In some embodiments, the siRNA molecule comprises a modified sense strand sequence of SEQ ID NO. 148 and a modified antisense strand sequence of SEQ ID NO. 358. In some embodiments, the siRNA molecule comprises a modified sense strand sequence of SEQ ID NO. 149 and a modified antisense strand sequence of SEQ ID NO. 359. In some embodiments, the siRNA molecule comprises a modified sense strand sequence of SEQ ID NO. 150 and a modified antisense strand sequence of SEQ ID NO. 360. In some embodiments, the siRNA molecule comprises a modified sense strand sequence of SEQ ID NO. 151 and a modified antisense strand sequence of SEQ ID NO. 361. In some embodiments, the siRNA molecule comprises a modified sense strand sequence of SEQ ID NO. 152 and a modified antisense strand sequence of SEQ ID NO. 362. In some embodiments, the siRNA molecule comprises a modified sense strand sequence of SEQ ID NO. 153 and a modified antisense strand sequence of SEQ ID NO. 363. In some embodiments, the siRNA molecule comprises a modified sense strand sequence of SEQ ID NO. 154 and a modified antisense strand sequence of SEQ ID NO. 364. In some embodiments, the siRNA molecule comprises a modified sense strand sequence of SEQ ID NO. 155 and a modified antisense strand sequence of SEQ ID NO. 365. In some embodiments, the siRNA molecule comprises a modified sense strand sequence of SEQ ID NO. 156 and a modified antisense strand sequence of SEQ ID NO. 366. In some embodiments, the siRNA molecule comprises a modified sense strand sequence of SEQ ID NO. 157 and a modified antisense strand sequence of SEQ ID NO. 367. In some embodiments, the siRNA molecule comprises a modified sense strand sequence of SEQ ID NO. 158 and a modified antisense strand sequence of SEQ ID NO. 368. In some embodiments, the siRNA molecule comprises a modified sense strand sequence of SEQ ID NO. 159 and a modified antisense strand sequence of SEQ ID NO. 369. In some embodiments, the siRNA molecule comprises a modified sense strand sequence of SEQ ID NO. 160 and a modified antisense strand sequence of SEQ ID NO. 370. In some embodiments, the siRNA molecule comprises a modified sense strand sequence of SEQ ID NO. 161 and a modified antisense strand sequence of SEQ ID NO. 371. In some embodiments, the siRNA molecule comprises a modified sense strand sequence of SEQ ID NO. 162 and a modified antisense strand sequence of SEQ ID NO. 372. In some embodiments, the siRNA molecule comprises a modified sense strand sequence of SEQ ID NO. 163 and a modified antisense strand sequence of SEQ ID NO. 373. In some embodiments, the siRNA molecule comprises a modified sense strand sequence of SEQ ID NO. 164 and a modified antisense strand sequence of SEQ ID NO. 374. In some embodiments, the siRNA molecule comprises a modified sense strand sequence of SEQ ID NO. 165 and a modified antisense strand sequence of SEQ ID NO. 375. In some embodiments, the siRNA molecule comprises a modified sense strand sequence of SEQ ID NO. 166 and a modified antisense strand sequence of SEQ ID NO. 376. In some embodiments, the siRNA molecule comprises a modified sense strand sequence of SEQ ID NO. 167 and a modified antisense strand sequence of SEQ ID NO. 377. In some embodiments, the siRNA molecule comprises a modified sense strand sequence of SEQ ID NO. 168 and a modified antisense strand sequence of SEQ ID NO. 378. In some embodiments, the siRNA molecule comprises a modified sense strand sequence of SEQ ID NO. 169 and a modified antisense strand sequence of SEQ ID NO. 379. In some embodiments, the siRNA molecule comprises a modified sense strand sequence of SEQ ID NO. 170 and a modified antisense strand sequence of SEQ ID NO. 380. In some embodiments, the siRNA molecule comprises a modified sense strand sequence of SEQ ID NO. 171 and a modified antisense strand sequence of SEQ ID NO. 381. In some embodiments, the siRNA molecule comprises a modified sense strand sequence of SEQ ID NO. 172 and a modified antisense strand sequence of SEQ ID NO. 382. In some embodiments, the siRNA molecule comprises a modified sense strand sequence of SEQ ID NO. 173 and a modified antisense strand sequence of SEQ ID NO. 383. In some embodiments, the siRNA molecule comprises a modified sense strand sequence of SEQ ID NO. 174 and a modified antisense strand sequence of SEQ ID NO. 384. In some embodiments, the siRNA molecule comprises a modified sense strand sequence of SEQ ID NO. 175 and a modified antisense strand sequence of SEQ ID NO. 385. In some embodiments, the siRNA molecule comprises a modified sense strand sequence of SEQ ID NO. 176 and a modified antisense strand sequence of SEQ ID NO. 386. In some embodiments, the siRNA molecule comprises a modified sense strand sequence of SEQ ID NO. 177 and a modified antisense strand sequence of SEQ ID NO. 387. In some embodiments, the siRNA molecule comprises a modified sense strand sequence of SEQ ID NO. 178 and a modified antisense strand sequence of SEQ ID NO. 388. In some embodiments, the siRNA molecule comprises a modified sense strand sequence of SEQ ID NO. 179 and a modified antisense strand sequence of SEQ ID NO. 389. In some embodiments, the siRNA molecule comprises a modified sense strand sequence of SEQ ID NO. 180 and a modified antisense strand sequence of SEQ ID NO. 390. In some embodiments, the siRNA molecule comprises a modified sense strand sequence of SEQ ID NO. 181 and a modified antisense strand sequence of SEQ ID NO. 391. In some embodiments, the siRNA molecule comprises a modified sense strand sequence of SEQ ID NO. 182 and a modified antisense strand sequence of SEQ ID NO. 392. In some embodiments, the siRNA molecule comprises a modified sense strand sequence of SEQ ID NO. 183 and a modified antisense strand sequence of SEQ ID NO. 393. In some embodiments, the siRNA molecule comprises a modified sense strand sequence of SEQ ID NO. 184 and a modified antisense strand sequence of SEQ ID NO. 394. In some embodiments, the siRNA molecule comprises a modified sense strand sequence of SEQ ID NO. 185 and a modified antisense strand sequence of SEQ ID NO. 395. In some embodiments, the siRNA molecule comprises a modified sense strand sequence of SEQ ID NO. 186 and a modified antisense strand sequence of SEQ ID NO. 396. In some embodiments, the siRNA molecule comprises a modified sense strand sequence of SEQ ID NO. 187 and a modified antisense strand sequence of SEQ ID NO. 397. In some embodiments, the siRNA molecule comprises a modified sense strand sequence of SEQ ID NO. 188 and a modified antisense strand sequence of SEQ ID NO. 398. In some embodiments, the siRNA molecule comprises a modified sense strand sequence of SEQ ID NO. 189 and a modified antisense strand sequence of SEQ ID NO. 399. In some embodiments, the siRNA molecule comprises a modified sense strand sequence of SEQ ID NO. 190 and a modified antisense strand sequence of SEQ ID NO. 400. In some embodiments, the siRNA molecule comprises a modified sense strand sequence of SEQ ID NO. 191 and a modified antisense strand sequence of SEQ ID NO. 401. In some embodiments, the siRNA molecule comprises a modified sense strand sequence of SEQ ID NO. 192 and a modified antisense strand sequence of SEQ ID NO. 402. In some embodiments, the siRNA molecule comprises a modified sense strand sequence of SEQ ID NO. 193 and a modified antisense strand sequence of SEQ ID NO. 403. In some embodiments, the siRNA molecule comprises a modified sense strand sequence of SEQ ID NO. 194 and a modified antisense strand sequence of SEQ ID NO. 404. In some embodiments, the siRNA molecule comprises a modified sense strand sequence of SEQ ID NO. 195 and a modified antisense strand sequence of SEQ ID NO. 405. In some embodiments, the siRNA molecule comprises a modified sense strand sequence of SEQ ID NO. 196 and a modified antisense strand sequence of SEQ ID NO. 406. In some embodiments, the siRNA molecule comprises a modified sense strand sequence of SEQ ID NO. 197 and a modified antisense strand sequence of SEQ ID NO. 407. In some embodiments, the siRNA molecule comprises a modified sense strand sequence of SEQ ID NO. 198 and a modified antisense strand sequence of SEQ ID NO. 408. In some embodiments, the siRNA molecule comprises a modified sense strand sequence of SEQ ID NO. 199 and a modified antisense strand sequence of SEQ ID NO. 409. In some embodiments, the siRNA molecule comprises a modified sense strand sequence of SEQ ID NO. 200 and a modified antisense strand sequence of SEQ ID NO. 410. In some embodiments, the siRNA molecule comprises a modified sense strand sequence of SEQ ID NO. 201 and a modified antisense strand sequence of SEQ ID NO. 411. In some embodiments, the siRNA molecule comprises a modified sense strand sequence of SEQ ID NO. 202 and a modified antisense strand sequence of SEQ ID NO. 412. In some embodiments, the siRNA molecule comprises a modified sense strand sequence of SEQ ID NO. 203 and a modified antisense strand sequence of SEQ ID NO. 413. In some embodiments, the siRNA molecule comprises a modified sense strand sequence of SEQ ID NO. 204 and a modified antisense strand sequence of SEQ ID NO. 414. In some embodiments, the siRNA molecule comprises a modified sense strand sequence of SEQ ID NO. 205 and a modified antisense strand sequence of SEQ ID NO. 415. In some embodiments, the siRNA molecule comprises a modified sense strand sequence of SEQ ID NO. 206 and a modified antisense strand sequence of SEQ ID NO. 416. In some embodiments, the siRNA molecule comprises a modified sense strand sequence of SEQ ID NO. 207 and a modified antisense strand sequence of SEQ ID NO. 417. In some embodiments, the siRNA molecule comprises a modified sense strand sequence of SEQ ID NO. 208 and a modified antisense strand sequence of SEQ ID NO. 418. In some embodiments, the siRNA molecule comprises a modified sense strand sequence of SEQ ID NO. 209 and a modified antisense strand sequence of SEQ ID NO. 419. In some embodiments, the siRNA molecule comprises a modified sense strand sequence of SEQ ID NO. 210 and a modified antisense strand sequence of SEQ ID NO. 420. In some embodiments, the siRNA molecule comprises a modified sense strand sequence of SEQ ID NO. 211 and a modified antisense strand sequence of SEQ ID NO. 421. In some embodiments, the siRNA molecule comprises a modified sense strand sequence of SEQ ID NO. 212 and a modified antisense strand sequence of SEQ ID NO. 422. In some embodiments, the siRNA molecule comprises a modified sense strand sequence of SEQ ID NO. 213 and a modified antisense strand sequence of SEQ ID NO. 423. In some embodiments, the siRNA molecule comprises a modified sense strand sequence of SEQ ID NO. 214 and a modified antisense strand sequence of SEQ ID NO. 424. In some embodiments, the siRNA molecule comprises a modified sense strand sequence of SEQ ID NO. 215 and a modified antisense strand sequence of SEQ ID NO. 425. In some embodiments, the siRNA molecule comprises a modified sense strand sequence of SEQ ID NO. 216 and a modified antisense strand sequence of SEQ ID NO. 426. In some embodiments, the siRNA molecule comprises a modified sense strand sequence of SEQ ID NO. 217 and a modified antisense strand sequence of SEQ ID NO. 427. In some embodiments, the siRNA molecule comprises a modified sense strand sequence of SEQ ID NO. 218 and a modified antisense strand sequence of SEQ ID NO. 428. In some embodiments, the siRNA molecule comprises a modified sense strand sequence of SEQ ID NO. 219 and a modified antisense strand sequence of SEQ ID NO. 429. In some embodiments, the siRNA molecule comprises a modified sense strand sequence of SEQ ID NO. 220 and a modified antisense strand sequence of SEQ ID NO. 430. In some embodiments, the siRNA molecule comprises a modified sense strand sequence of SEQ ID NO. 221 and a modified antisense strand sequence of SEQ ID NO. 431. In some embodiments, the siRNA molecule comprises a modified sense strand sequence of SEQ ID NO. 222 and a modified antisense strand sequence of SEQ ID NO. 432. In some embodiments, the siRNA molecule comprises a modified sense strand sequence of SEQ ID NO. 223 and a modified antisense strand sequence of SEQ ID NO. 433. In some embodiments, the siRNA molecule comprises a modified sense strand sequence of SEQ ID NO. 224 and a modified antisense strand sequence of SEQ ID NO. 434. In some embodiments, the siRNA molecule comprises a modified sense strand sequence of SEQ ID NO. 225 and a modified antisense strand sequence of SEQ ID NO. 435. In some embodiments, the siRNA molecule comprises a modified sense strand sequence of SEQ ID NO. 226 and a modified antisense strand sequence of SEQ ID NO. 436. In some embodiments, the siRNA molecule comprises a modified sense strand sequence of SEQ ID NO. 227 and a modified antisense strand sequence of SEQ ID NO. 437. In some embodiments, the siRNA molecule comprises a modified sense strand sequence of SEQ ID NO. 228 and a modified antisense strand sequence of SEQ ID NO. 438. In some embodiments, the siRNA molecule comprises a modified sense strand sequence of SEQ ID NO. 229 and a modified antisense strand sequence of SEQ ID NO. 439. In some embodiments, the siRNA molecule comprises a modified sense strand sequence of SEQ ID NO. 230 and a modified antisense strand sequence of SEQ ID NO. 440. In some embodiments, the siRNA molecule comprises a modified sense strand sequence of SEQ ID NO. 231 and a modified antisense strand sequence of SEQ ID NO. 441. In some embodiments, the siRNA molecule comprises a modified sense strand sequence of SEQ ID NO. 232 and a modified antisense strand sequence of SEQ ID NO. 442. In some embodiments, the siRNA molecule comprises a modified sense strand sequence of SEQ ID NO. 233 and a modified antisense strand sequence of SEQ ID NO. 443. In some embodiments, the siRNA molecule comprises a modified sense strand sequence of SEQ ID NO. 234 and a modified antisense strand sequence of SEQ ID NO. 444. In some embodiments, the siRNA molecule comprises a modified sense strand sequence of SEQ ID NO. 235 and a modified antisense strand sequence of SEQ ID NO. 445. In some embodiments, the siRNA molecule comprises a modified sense strand sequence of SEQ ID NO. 236 and a modified antisense strand sequence of SEQ ID NO. 446. In some embodiments, the siRNA molecule comprises a modified sense strand sequence of SEQ ID NO. 237 and a modified antisense strand sequence of SEQ ID NO. 447. In some embodiments, the siRNA molecule comprises a modified sense strand sequence of SEQ ID NO. 238 and a modified antisense strand sequence of SEQ ID NO. 448. In some embodiments, the siRNA molecule comprises a modified sense strand sequence of SEQ ID NO. 239 and a modified antisense strand sequence of SEQ ID NO. 449. In some embodiments, the siRNA molecule comprises a modified sense strand sequence of SEQ ID NO. 240 and a modified antisense strand sequence of SEQ ID NO. 450. In some embodiments, the siRNA molecule comprises a modified sense strand sequence of SEQ ID NO. 241 and a modified antisense strand sequence of SEQ ID NO. 451. In some embodiments, the siRNA molecule comprises a modified sense strand sequence of SEQ ID NO. 242 and a modified antisense strand sequence of SEQ ID NO. 452. In some embodiments, the siRNA molecule comprises a modified sense strand sequence of SEQ ID NO. 243 and a modified antisense strand sequence of SEQ ID NO. 453. In some embodiments, the siRNA molecule comprises a modified sense strand sequence of SEQ ID NO. 244 and a modified antisense strand sequence of SEQ ID NO. 454. In some embodiments, the siRNA molecule comprises a modified sense strand sequence of SEQ ID NO. 245 and a modified antisense strand sequence of SEQ ID NO. 455. In some embodiments, the siRNA molecule comprises a modified sense strand sequence of SEQ ID NO. 246 and a modified antisense strand sequence of SEQ ID NO. 456. In some embodiments, the siRNA molecule comprises a modified sense strand sequence of SEQ ID NO. 247 and a modified antisense strand sequence of SEQ ID NO. 457. In some embodiments, the siRNA molecule comprises a modified sense strand sequence of SEQ ID NO. 248 and a modified antisense strand sequence of SEQ ID NO. 458. In some embodiments, the siRNA molecule comprises a modified sense strand sequence of SEQ ID NO. 249 and a modified antisense strand sequence of SEQ ID NO. 459. In some embodiments, the siRNA molecule comprises a modified sense strand sequence of SEQ ID NO. 250 and a modified antisense strand sequence of SEQ ID NO. 460. In some embodiments, the siRNA molecule comprises a modified sense strand sequence of SEQ ID NO. 251 and a modified antisense strand sequence of SEQ ID NO. 461. In some embodiments, the siRNA molecule comprises a modified sense strand sequence of SEQ ID NO. 252 and a modified antisense strand sequence of SEQ ID NO. 462. In some embodiments, the siRNA molecule comprises a modified sense strand sequence of SEQ ID NO. 253 and a modified antisense strand sequence of SEQ ID NO. 463. In some embodiments, the siRNA molecule comprises a modified sense strand sequence of SEQ ID NO. 254 and a modified antisense strand sequence of SEQ ID NO. 464. In some embodiments, the siRNA molecule comprises a modified sense strand sequence of SEQ ID NO. 255 and a modified antisense strand sequence of SEQ ID NO. 465. In some embodiments, the siRNA molecule comprises a modified sense strand sequence of SEQ ID NO. 256 and a modified antisense strand sequence of SEQ ID NO. 466. In some embodiments, the siRNA molecule comprises a modified sense strand sequence of SEQ ID NO. 257 and a modified antisense strand sequence of SEQ ID NO. 467. In some embodiments, the siRNA molecule comprises a modified sense strand sequence of SEQ ID NO. 258 and a modified antisense strand sequence of SEQ ID NO. 468. In some embodiments, the siRNA molecule comprises a modified sense strand sequence of SEQ ID NO. 259 and a modified antisense strand sequence of SEQ ID NO. 469. In some embodiments, the siRNA molecule comprises a modified sense strand sequence of SEQ ID NO. 260 and a modified antisense strand sequence of SEQ ID NO. 470. In some embodiments, the siRNA molecule comprises a modified sense strand sequence of SEQ ID NO. 261 and a modified antisense strand sequence of SEQ ID NO. 471. In some embodiments, the siRNA molecule comprises a modified sense strand sequence of SEQ ID NO. 262 and a modified antisense strand sequence of SEQ ID NO. 472. In some embodiments, the siRNA molecule comprises a modified sense strand sequence of SEQ ID NO. 263 and a modified antisense strand sequence of SEQ ID NO. 473. In some embodiments, the siRNA molecule comprises a modified sense strand sequence of SEQ ID NO. 264 and a modified antisense strand sequence of SEQ ID NO. 474. In some embodiments, the siRNA molecule comprises a modified sense strand sequence of SEQ ID NO. 265 and a modified antisense strand sequence of SEQ ID NO. 475. In some embodiments, the siRNA molecule comprises a modified sense strand sequence of SEQ ID NO. 266 and a modified antisense strand sequence of SEQ ID NO. 476. In some embodiments, the siRNA molecule comprises a modified sense strand sequence of SEQ ID NO. 267 and a modified antisense strand sequence of SEQ ID NO. 477. In some embodiments, the siRNA molecule comprises a modified sense strand sequence of SEQ ID NO. 268 and a modified antisense strand sequence of SEQ ID NO. 478. In some embodiments, the siRNA molecule comprises a modified sense strand sequence of SEQ ID NO. 269 and a modified antisense strand sequence of SEQ ID NO. 479. In some embodiments, the siRNA molecule comprises a modified sense strand sequence of SEQ ID NO. 270 and a modified antisense strand sequence of SEQ ID NO. 480.

TABLE 3
Exemplary modified sense and antisense strand sequences of siRNAs

| | | GalN | |
|---|---|---|---|
| | | Ac at | |
| | | the 3′ | |
| | | end of | |
| Duplex | Sense (S) | the S | Antisense strand (AS) |
| ID | strand sequence (5′-3′) | strand | sequence (5′-3′) |
| SR271 | asgsauCfaAfGfGfcucacuuaua | L96 | usAfsuaaGfugagccuUfgAfucususg |
| SR442 | (SEQ ID NO. 61) | AWH | (SEQ ID NO. 271) |
| SR195 | uscsagCfaUfUfGfugaugaccaa | L96 | usUfsgguCfaucacaaUfgCfugasgsg |
| SR499 | (SEQ ID NO. 62) | AWH | (SEQ ID NO. 272) |
| SR240 | uscsauCfaUfUfCfucuugugcaa | L96 | usUfsgcaCfaagagaaUfgAfugasusc |
| SR364 | (SEQ ID NO. 63) | AWH | (SEQ ID NO. 273) |
| SR028 | usgsgcCfgUfCfAfcaguucucua | L96 | usAfsgagAfacugugaCfgGfccascsa |
| SR470 | (SEQ ID NO. 64) | AWH | (SEQ ID NO. 274) |
| SR167 | usgsguCfcCfAfAfagcaauucua | L96 | usAfsgaaUfugcuuugGfgAfccasgsa |
| SR651 | (SEQ ID NO. 65) | AWH | (SEQ ID NO. 275) |
| SR082 | uscscaGfuAfCfAfucuaucagaa | L96 | usUfscugAfuagauguAfcUfggasasa |
| SR597 | (SEQ ID NO. 66) | AWH | (SEQ ID NO. 276) |
| SR197 | cscsagUfaCfAfUfcuaucagaaa | L96 | usUfsucuGfauagaugUfaCfuggsasa |
| SR700 | (SEQ ID NO. 67) | AWH | (SEQ ID NO. 277) |
| SR158 | asuscuAfuCfAfGfaaucaucaaa | L96 | usUfsugaUfgauucugAfuAfgausgsu |
| SR402 | (SEQ ID NO. 68) | AWH | (SEQ ID NO. 278) |
| SR019 | asasguCfaCfUfCfaauuccugaa | L96 | usUfscagGfaauugagUfgAfcuususa |
| SR640 | (SEQ ID NO. 69) | AWH | (SEQ ID NO. 279) |
| SR237 | cscsugAfaCfUfAfaucaauauaa | L96 | usUfsauaUfugauuagUfuCfaggsasa |
| SR534 | (SEQ ID NO. 70) | AWH | (SEQ ID NO. 280) |
| SR259 | usgsaaCfuAfAfUfcaauauauaa | L96 | usUfsauaUfauugauuAfgUfucasgsg |
| SR376 | (SEQ ID NO. 71) | AWH | (SEQ ID NO. 281) |
| SR047 | usgsguUfcUfCfAfuuccuugcaa | L96 | usUfsgcaAfggaaugaGfaAfccasgsg |
| SR370 | (SEQ ID NO. 72) | AWH | (SEQ ID NO. 282) |
| SR159 | csuscaUfuCfCfUfugcaccauaa | L96 | usUfsaugGfugcaaggAfaUfgagsasa |
| SR474 | (SEQ ID NO. 73) | AWH | (SEQ ID NO. 283) |
| SR332 | ususggCfuUfUfCfugcuggguua | L96 | usAfsaccCfagcagaaAfgCfcaasusa |
| SR624 | (SEQ ID NO. 74) | AWH | (SEQ ID NO. 284) |
| SR297 | uscscuCfuAfCfAfugauuuucca | L96 | usGfsgaaAfaucauguAfgAfggasgsg |
| SR622 | (SEQ ID NO. 75) | AWH | (SEQ ID NO. 285) |
| SR244 | asgsAfucaAfGfGfcuCfacuuAfua | L96 | (vinu)sAfsuaagugagccuUfgaucususg |
| SR496 | (SEQ ID NO. 76) | AWH | (SEQ ID NO. 286) |
| SR176 | uscsAfgcaUfUfGfugAfugacCfaa | L96 | (vinu)sUfsggucaucacaaUfgcugasgsg |
| SR529 | (SEQ ID NO. 77) | AWH | (SEQ ID NO. 287) |
| SR053 | uscsAfucaUfUfCfucUfugugCfaa | L96 | (vinu)sUfsgcacaagagaaUfgaugasusc |
| SR507 | (SEQ ID NO. 78) | AWH | (SEQ ID NO. 288) |
| SR107 | usgsGfccgUfCfAfcaGfuucuCfua | L96 | (vinu)sAfsgagaacugugaCfggccascsa |
| SR584 | (SEQ ID NO. 79) | AWH | (SEQ ID NO. 289) |
| SR056 | usgsGfuccCfAfAfagCfaauuCfua | L96 | (vinu)sAfsgaauugcuuugGfgaccasgsa |
| SR571 | (SEQ ID NO. 80) | AWH | (SEQ ID NO. 290) |
| SR238 | uscsCfaguAfCfAfucUfaucaGfaa | L96 | (vinu)sUfscugauagauguAfcuggasasa |
| SR601 | (SEQ ID NO. 81) | AWH | (SEQ ID NO. 291) |
| SR323 | cscsAfguaCfAfUfcuAfucagAfaa | L96 | (vinu)sUfsucugauagaugUfacuggsasa |
| SR407 | (SEQ ID NO. 82) | AWH | (SEQ ID NO. 292) |
| SR009 | asusCfuauCfAfGfaaUfcaucAfaa | L96 | (vinu)sUfsugaugauucugAfuagausgsu |
| SR562 | (SEQ ID NO. 83) | AWH | (SEQ ID NO. 293) |
| SR316 | asasGfucaCfUfCfaaUfuccuGfaa | L96 | (vinu)sUfscaggaauugagUfgacuususa |
| SR600 | (SEQ ID NO. 84) | AWH | (SEQ ID NO. 294) |
| SR133 | cscsUfgaaCfUfAfauCfaauaUfaa | L96 | (vinu)sUfsauauugauuagUfucaggsasa |
| SR581 | (SEQ ID NO. 85) | AWH | (SEQ ID NO. 295) |
| SR058 | usgsAfacuAfAfUfcaAfuauaUfaa | L96 | (vinu)sUfsauauauugauuAfguucasgsg |
| SR438 | (SEQ ID NO. 86) | AWH | (SEQ ID ID NO. 296) |
| SR230 | usgsGfuucUfCfAfuuCfcuugCfaa | L96 | (vinu)sUfsgcaaggaaugaGfaaccasgsg |
| SR569 | (SEQ ID NO. 87) | AWH | (SEQ ID NO. 297) |
| SR322 | csusCfauuCfCfUfugCfaccaUfaa | L96 | (vinu)sUfsauggugcaaggAfaugagsasa |
| SR625 | (SEQ ID NO. 88) | AWH | (SEQ ID ID NO. 298) |
| SR125 | ususGfgcuUfUfCfugCfugggUfua | L96 | (vinu)sAfsacccagcagaaAfgccaasusa |
| SR373 | (SEQ ID NO. 89) | AWH | (SEQ ID NO. 299) |
| SR256 | uscsCfucuAfCfAfugAfuuuuCfca | L96 | (vinu)sGfsgaaaaucauguAfgaggasgsg |
| SR476 | (SEQ ID NO. 90) | AWH | (SEQ ID NO. 300) |
| SR187 | asgsauCfaAfGfGfcucacuuaua | L96 | (vinu)sAfsuaaGfuGfAfgccuUfgAfucususg |
| SR473 | (SEQ ID NO. 91) | AWH | (SEQ ID NO. 301) |
| SR150 | uscsagCfaUfUfGfugaugaccaa | L96 | (vinu)sUfsgguCfaUfCfacaaUfgCfugasgsg |
| SR639 | (SEQ ID NO. 92) | AWH | (SEQ ID NO. 302) |
| SR295 | uscsauCfaUfUfCfucuugugcaa | L96 | (vinu)sUfsgcaCfaAfGfagaaUfgAfugasusc |
| SR703 | (SEQ ID NO. 93) | AWH | (SEQ ID NO. 303) |
| SR148 | usgsgcCfgUfCfAfcaguucucua | L96 | (vinu)sAfsgagAfaCfUfgugaCfgGfccascsa |
| SR528 | (SEQ ID NO. 94) | AWH | (SEQ ID NO. 304) |
| SR131 | usgsguCfcCfAfAfagcaauucua | L96 | (vinu)sAfsgaaUfuGfCfuuugGfgAfccasgsa |
| SR702 | (SEQ ID NO. 95) | AWH | (SEQ ID NO. 305) |
| SR304 | uscscaGfuAfCfAfucuaucagaa | L96 | (vinu)sUfscugAfuAfGfauguAfcUfggasasa |
| SR559 | (SEQ ID NO. 96) | AWH | (SEQ ID NO. 306) |
| SR266 | cscsagUfaCfAfUfcuaucagaaa | L96 | (vinu)sUfsucuGfaUfAfgaugUfaCfuggsasa |
| SR521 | (SEQ ID NO. 97) | AWH | (SEQ ID NO. 307) |
| SR232 | asuscuAfuCfAfGfaaucaucaaa | L96 | (vinu)sUfsugaUfgAfUfucugAfuAfgausgsu |
| SR439 | (SEQ ID NO. 98) | AWH | (SEQ ID NO. 308) |
| SR233 | asasguCfaCfUfCfaauuccugaa | L96 | (vinu)sUfscagGfaAfUfugagUfgAfcuususa |
| SR642 | (SEQ ID NO. 99) | AWH | (SEQ ID NO. 309) |
| SR073 | cscsugAfaCfUfAfaucaauauaa | L96 | (vinu)sUfsauaUfuGfAfuuagUfuCfaggsasa |
| SR480 | (SEQ ID NO. 100) | AWH | (SEQ ID NO. 310) |
| SR038 | usgsaaCfuAfAfUfcaauauauaa | L96 | (vinu)sUfsauaUfaUfUfgauuAfgUfucasgsg |
| SR643 | (SEQ ID NO. 101) | AWH | (SEQ ID NO. 311) |
| SR257 | usgsguUfcUfCfAfuuccuugcaa | L96 | (vinu)sUfsgcaAfgGfAfaugaGfaAfccasgsg |
| SR484 | (SEQ ID NO. 102) | AWH | (SEQ ID NO. 312) |
| SR141 | csuscaUfuCfCfUfugcaccauaa | L96 | (vinu)sUfsaugGfuGfCfaaggAfaUfgagsasa |
| SR358 | (SEQ ID NO. 103) | AWH | (SEQ ID NO. 313) |
| SR138 | ususggCfuUfUfCfugcuggguua | L96 | (vinu)sAfsaccCfaGfCfagaaAfgCfcaasusa |
| SR630 | (SEQ ID NO. 104) | AWH | (SEQ ID NO. 314) |
| SR300 | uscscuCfuAfCfAfugauuuucca | L96 | (vinu)sGfsgaaAfaUfCfauguAfgAfggasgsg |
| SR513 | (SEQ ID NO. 105) | AWH | (SEQ ID NO. 315) |
| SR052 | asgsauCfaAfGfGfcucaCfuuAfua | L96 | (vinu)sAfsuaaGfuGfAfgccuUfgAfucususg |
| SR465 | (SEQ ID NO. 106) | AWH | (SEQ ID NO. 316) |
| SR163 | uscsagCfaUfUfGfugauGfacCfaa | L96 | (vinu)sUfsgguCfaUfCfacaaUfgCfugasgsg |
| SR563 | (SEQ ID NO. 107) | AWH | (SEQ ID NO. 317) |
| SR078 | uscsauCfaUfUfCfucuuGfugCfaa | L96 | (vinu)sUfsgcaCfaAfGfagaaUfgAfugasusc |
| SR690 | (SEQ ID NO. 108) | AWH | (SEQ ID NO. 318) |
| SR296 | usgsgcCfgUfCfAfcaguUfcuCfua | L96 | (vinu)sAfsgagAfaCfUfgugaCfgGfccascsa |
| SR366 | (SEQ ID NO. 109) | AWH | (SEQ ID NO. 319) |
| SR122 | usgsguCfcCfAfAfagcaAfuuCfua | L96 | (vinu)sAfsgaaUfuGfCfuuugGfgAfccasgsa |
| SR646 | (SEQ ID NO. 110) | AWH | (SEQ ID NO. 320) |
| SR248 | uscscaGfuAfCfAfucuaUfcaGfaa | L96 | (vinu)sUfscugAfuAfGfauguAfcUfggasasa |
| SR371 | (SEQ ID NO. 111) | AWH | (SEQ ID NO. 321) |
| SR292 | cscsagUfaCfAfUfcuauCfagAfaa | L96 | (vinu)sUfsucuGfaUfAfgaugUfaCfuggsasa |
| SR580 | (SEQ ID NO. 112) | AWH | (SEQ ID NO. 322) |
| SR178 | asuscuAfuCfAfGfaaucAfucAfaa | L96 | (vinu)sUfsugaUfgAfUfucugAfuAfgausgsu |
| SR633 | (SEQ ID NO. 113) | AWH | (SEQ ID NO. 323) |
| SR063 | asasguCfaCfUfCfaauuCfcuGfaa | L96 | (vinu)sUfscagGfaAfUfugagUfgAfcuususa |
| SR552 | (SEQ ID NO. 114) | AWH | (SEQ ID NO. 324) |
| SR338 | cscsugAfaCfUfAfaucaAfuaUfaa | L96 | (vinu)sUfsauaUfuGfAfuuagUfuCfaggsasa |
| SR684 | (SEQ ID NO. 115) | AWH | (SEQ ID NO. 325) |
| SR098 | usgsaaCfuAfAfUfcaauAfuaUfaa | L96 | (vinu)sUfsauaUfaUfUfgauuAfgUfucasgsg |
| SR683 | (SEQ ID NO. 116) | AWH | (SEQ ID NO. 326) |
| SR044 | usgsguUfcUfCfAfuuccUfugCfaa | L96 | (vinu)sUfsgcaAfgGfAfaugaGfaAfccasgsg |
| SR707 | (SEQ ID NO. 117) | AWH | (SEQ ID NO. 327) |
| SR200 | csuscaUfuCfCfUfugcaCfcaUfaa | L96 | (vinu)sUfsaugGfuGfCfaaggAfaUfgagsasa |
| SR679 | (SEQ ID NO. 118) | AWH | (SEQ ID NO. 328) |
| SR325 | ususggCfuUfUfCfugcuGfggUfua | L96 | (vinu)sAfsaccCfaGfCfagaaAfgCfcaasusa |
| SR567 | (SEQ ID NO. 119) | AWH | (SEQ ID NO. 329) |
| SR071 | uscscuCfuAfCfAfugauUfuuCfca | L96 | (vinu)sGfsgaaAfaUfCfauguAfgAfggasgsg |
| SR356 | (SEQ ID NO. 120) | AWH | (SEQ ID NO. 330) |
| SR264 | asgsauCfaAfGfGfcucacuuaua | L96 | (vinu)sAfsuaaGfugagccuUfgAfucususg |
| SR532 | (SEQ ID NO. 121) | AWH | (SEQ ID NO. 331) |
| SR319 | uscsagCfaUfUfGfugaugaccaa | L96 | (vinu)sUfsgguCfaucacaaUfgCfugasgsg |
| SR477 | (SEQ ID NO. 122) | AWH | (SEQ ID NO. 332) |
| SR180 | uscsauCfaUfUfCfucuugugcaa | L96 | (vinu)sUfsgcaCfaagagaaUfgAfugasusc |
| SR696 | (SEQ ID NO. 123) | AWH | (SEQ ID NO. 333) |
| SR220 | usgsgcCfgUfCfAfcaguucucua | L96 | (vinu)sAfsgagAfacugugaCfgGfccascsa |
| SR447 | (SEQ ID NO. 124) | AWH | (SEQ ID NO. 334) |
| SR320 | usgsguCfcCfAfAfagcaauucua | L96 | (vinu)sAfsgaaUfugcuuugGfgAfccasgsa |
| SR698 | (SEQ ID NO. 125) | AWH | (SEQ ID NO. 335) |
| SR160 | uscscaGfuAfCfAfucuaucagaa | L96 | (vinu)sUfscugAfuagauguAfcUfggasasa |
| SR613 | (SEQ ID NO. 126) | AWH | (SEQ ID NO. 336) |
| SR168 | cscsagUfaCfAfUfcuaucagaaa | L96 | (vinu)sUfsucuGfauagaugUfaCfuggsasa |
| SR410 | (SEQ ID NO. 127) | AWH | (SEQ ID NO. 337) |
| SR006 | asuscuAfuCfAfGfaaucaucaaa | L96 | (vinu)sUfsugaUfgauucugAfuAfgausgsu |
| SR350 | (SEQ ID NO. 128) | AWH | (SEQ ID NO. 338) |
| SR255 | asasguCfaCfUfCfaauuccugaa | L96 | (vinu)sUfscagGfaauugagUfgAfcuususa |
| SR454 | (SEQ ID NO. 129) | AWH | (SEQ ID NO. 339) |
| SR184 | cscsugAfaCfUfAfaucaauauaa | L96 | (vinu)sUfsauaUfugauuagUfuCfaggsasa |
| SR504 | (SEQ ID NO. 130) | AWH | (SEQ ID NO. 340) |
| SR083 | usgsaaCfuAfAfUfcaauauauaa | L96 | (vinu)sUfsauaUfauugauuAfgUfucasgsg |
| SR697 | (SEQ ID NO. 131) | AWH | (SEQ ID NO. 341) |
| SR031 | usgsguUfcUfCfAfuuccuugcaa | L96 | (vinu)sUfsgcaAfggaaugaGfaAfccasgsg |
| SR419 | (SEQ ID NO. 132) | AWH | (SEQ ID NO. 342) |
| SR020 | csuscaUfuCfCfUfugcaccauaa | L96 | (vinu)sUfsaugGfugcaaggAfaUfgagsasa |
| SR596 | (SEQ ID NO. 133) | AWH | (SEQ ID NO. 343) |
| SR286 | ususggCfuUfUfCfugcuggguua | L96 | (vinu)sAfsaccCfagcagaaAfgCfcaasusa |
| SR682 | (SEQ ID NO. 134) | AWH | (SEQ ID NO. 344) |
| SR283 | uscscuCfuAfCfAfugauuuucca | L96 | (vinu)sGfsgaaAfaucauguAfgAfggasgsg |
| SR687 | (SEQ ID NO. 135) | AWH | (SEQ ID NO. 345) |
| SR274 | csasagauCfaAfGfGfcucacuuaua | L96 | (vinu)sAfsuaaGfuGfAfgccuUfgAfucuugscsu |
| SR424 | (SEQ ID NO. 136) | AWH | (SEQ ID NO. 346) |
| SR208 | cscsucagCfaUfUfGfugaugaccaa | L96 | (vinu)sUfsgguCfaUfCfacaaUfgCfugaggsusu |
| SR448 | (SEQ ID NO. 137) | AWH | (SEQ ID NO. 347) |
| SR260 | gsasucauCfaUfUfCfucuugugcaa | L96 | (vinu)sUfsgcaCfaAfGfagaaUfgAfugaucsasu |
| SR565 | (SEQ ID NO. 138) | AWH | (SEQ ID NO. 348) |
| SR017 | usgsuggcCfgUfCfAfcaguucucua | L96 | (vinu)sAfsgagAfaCfUfgugaCfgGfccacascsu |
| SR414 | (SEQ ID NO. 139) | AWH | (SEQ ID NO. 349) |
| SR130 | uscsugguCfcCfAfAfagcaauucua | L96 | (vinu)sAfsgaaUfuGfCfuuugGfgAfccagasasu |
| SR487 | (SEQ ID NO. 140) | AWH | (SEQ ID NO. 350) |
| SR333 | ususuccaGfuAfCfAfucuaucagaa | L96 | (vinu)sUfscugAfuAfGfauguAfcUfggaaasusg |
| SR666 | (SEQ ID NO. 141) | AWH | (SEQ ID NO. 351) |
| SR321 | ususccagUfaCfAfUfcuaucagaaa | L96 | (vinu)sUfsucuGfaUfAfgaugUfaCfuggaasasu |
| SR603 | (SEQ ID NO. 142) | AWH | (SEQ ID NO. 352) |
| SR212 | ascsaucuAfuCfAfGfaaucaucaaa | L96 | (vinu)sUfsugaUfgAfUfucugAfuAfgaugusasc |
| SR676 | (SEQ ID NO. 143) | AWH | (SEQ ID NO. 353) |
| SR066 | usasaaguCfaCfUfCfaauuccugaa | L96 | (vinu)sUfscagGfaAfUfugagUfgAfcuuuasasg |
| SR475 | (SEQ ID NO. 144) | AWH | (SEQ ID NO. 354) |
| SR091 | ususccugAfaCfUfAfaucaauauaa | L96 | (vinu)sUfsauaUfuGfAfuuagUfuCfaggaasusu |
| SR375 | (SEQ ID NO. 145) | AWH | (SEQ ID NO. 355) |
| SR110 | cscsugaaCfuAfAfUfcaauauauaa | L96 | (vinu)sUfsauaUfaUfUfgauuAfgUfucaggsasa |
| SR518 | (SEQ ID NO. 146) | AWH | (SEQ ID NO. 356) |
| SR097 | cscsugguUfcUfCfAfuuccuugcaa | L96 | (vinu)sUfsgcaAfgGfAfaugaGfaAfccaggsasc |
| SR427 | (SEQ ID NO. 147) | AWH | (SEQ ID NO. 357) |
| SR205 | ususcucaUfuCfCfUfugcaccauaa | L96 | (vinu)sUfsaugGfuGfCfaaggAfaUfgagaascsc |
| SR445 | (SEQ ID NO. 148) | AWH | (SEQ ID NO. 358) |
| SR165 | usasuuggCfuUfUfCfugcuggguua | L96 | (vinu)sAfsaccCfaGfCfagaaAfgCfcaauasasa |
| SR678 | (SEQ ID NO. 149) | AWH | (SEQ ID NO. 359) |
| SR314 | cscsuccuCfuAfCfAfugauuuucca | L96 | (vinu)sGfsgaaAfaUfCfauguAfgAfggaggsgsg |
| SR608 | (SEQ ID NO. 150) | AWH | (SEQ ID NO. 360) |
| SR140 | csasagauCfaAfGfGfcucaCfuuAfua | L96 | (vinu)sAfsuaaGfuGfAfgccuUfgAfucuugscsu |
| SR649 | (SEQ ID NO. 151) | AWH | (SEQ ID NO. 361) |
| SR096 | cscsucagCfaUfUfGfugauGfacCfaa | L96 | (vinu)sUfsgguCfaUfCfacaaUfgCfugaggsusu |
| SR631 | (SEQ ID NO. 152) | AWH | (SEQ ID NO. 362) |
| SR106 | gsasucauCfaUfUfCfucuuGfugCfaa | L96 | (vinu)sUfsgcaCfaAfGfagaaUfgAfugaucsasu |
| SR526 | (SEQ ID NO. 153) | AWH | (SEQ ID NO. 363) |
| SR263 | usgsuggcCfgUfCfAfcaguUfcuCfu | L96 | (vinu)sAfsgagAfaCfUfgugaCfgGfccacascsu |
| SR355 | a | AWH | (SEQ ID NO. 364) |
| | (SEQ ID NO. 154) | | |
| SR222 | uscsugguCfcCfAfAfagcaAfuuCfua | L96 | (vinu)sAfsgaaUfuGfCfuuugGfgAfccagasasu |
| SR655 | (SEQ ID NO. 155) | AWH | (SEQ ID NO. 365) |
| SR294 | ususuccaGfuAfCfAfucuaUfcaGfaa | L96 | (vinu)sUfscugAfuAfGfauguAfcUfggaaasusg |
| SR450 | (SEQ ID NO. 156) | AWH | (SEQ ID NO. 366) |
| SR124 | ususccagUfaCfAfUfcuauCfagAfaa | L96 | (vinu)sUfsucuGfaUfAfgaugUfaCfuggaasasu |
| SR361 | (SEQ ID NO. 157) | AWH | (SEQ ID NO. 367) |
| SR111 | ascsaucuAfuCfAfGfaaucAfucAfaa | L96 | (vinu)sUfsugaUfgAfUfucugAfuAfgaugusasc |
| SR421 | (SEQ ID NO. 158) | AWH | (SEQ ID NO. 368) |
| SR326 | usasaaguCfaCfUfCfaauuCfcuGfaa | L96 | (vinu)sUfscagGfaAfUfugagUfgAfcuuuasasg |
| SR506 | (SEQ ID NO. 159) | AWH | (SEQ ID NO. 369) |
| SR253 | ususccugAfaCfUfAfaucaAfuaUfaa | L96 | (vinu)sUfsauaUfuGfAfuuagUfuCfaggaasusu |
| SR516 | (SEQ ID NO. 160) | AWH | (SEQ ID NO. 370) |
| SR291 | cscsugaaCfuAfAfUfcaauAfuaUfaa | L96 | (vinu)sUfsauaUfaUfUfgauuAfgUfucaggsasa |
| SR554 | (SEQ ID NO. 161) | AWH | (SEQ ID NO. 371) |
| SR011 | cscsugguUfcUfCfAfuuccUfugCfaa | L96 | (vinu)sUfsgcaAfgGfAfaugaGfaAfccaggsasc |
| SR374 | (SEQ ID NO. 162) | AWH | (SEQ ID NO. 372) |
| SR120 | ususcucaUfuCfCfUfugcaCfcaUfaa | L96 | (vinu)sUfsaugGfuGfCfaaggAfaUfgagaascsc |
| SR564 | (SEQ ID NO. 163) | AWH | (SEQ ID NO. 373) |
| SR269 | usasuuggCfuUfUfCfugcuGfggUfu | L96 | (vinu)sAfsaccCfaGfCfagaaAfgCfcaauasasa |
| SR661 | a | AWH | (SEQ ID NO. 374) |
| | (SEQ ID NO. 164) | | |
| SR042 | cscsuccuCfuAfCfAfugauUfuuCfca | L96 | (vinu)sGfsgaaAfaUfCfauguAfgAfggaggsgsg |
| SR384 | (SEQ ID NO. 165) | AWH | (SEQ ID NO. 375) |
| SR334 | csasagauCfaAfGfGfcucacuuaua | L96 | (vinu)sAfsuaaGfugagccuUfgAfucuugscsu |
| SR629 | (SEQ ID NO. 166) | AWH | (SEQ ID NO. 376) |
| SR228 | cscsucagCfaUfUfGfugaugaccaa | L96 | (vinu)sUfsgguCfaucacaaUfgCfugaggsusu |
| SR483 | (SEQ ID NO. 167) | AWH | (SEQ ID NO. 377) |
| SR345 | gsasucauCfaUfUfCfucuugugcaa | L96 | (vinu)sUfsgcaCfaagagaaUfgAfugaucsasu |
| SR527 | (SEQ ID NO. 168) | AWH | (SEQ ID NO. 378) |
| SR157 | usgsuggcCfgUfCfAfcaguucucua | L96 | (vinu)sAfsgagAfacugugaCfgGfccacascsu |
| SR556 | (SEQ ID NO. 169) | AWH | (SEQ ID NO. 379) |
| SR134 | uscsugguCfcCfAfAfagcaauucua | L96 | (vinu)sAfsgaaUfugcuuugGfgAfccagasasu |
| SR451 | (SEQ ID NO. 170) | AWH | (SEQ ID NO. 380) |
| SR041 | ususuccaGfuAfCfAfucuaucagaa | L96 | (vinu)sUfscugAfuagauguAfcUfggaaasusg |
| SR709 | (SEQ ID NO. 171) | AWH | (SEQ ID NO. 381) |
| SR278 | ususccagUfaCfAfUfcuaucagaaa | L96 | (vinu)sUfsucuGfauagaugUfaCfuggaasasu |
| SR605 | (SEQ ID NO. 172) | AWH | (SEQ ID NO. 382) |
| SR023 | ascsaucuAfuCfAfGfaaucaucaaa | L96 | (vinu)sUfsugaUfgauucugAfuAfgaugusasc |
| SR632 | (SEQ ID NO. 173) | AWH | (SEQ ID NO. 383) |
| SR245 | usasaaguCfaCfUfCfaauuccugaa | L96 | (vinu)sUfscagGfaauugagUfgAfcuuuasasg |
| SR543 | (SEQ ID NO. 174) | AWH | (SEQ ID NO. 384) |
| SR129 | ususccugAfaCfUfAfaucaauauaa | L96 | (vinu)sUfsauaUfugauuagUfuCfaggaasusu |
| SR430 | (SEQ ID NO. 175) | AWH | (SEQ ID NO. 385) |
| SR116 | cscsugaaCfuAfAfUfcaauauauaa | L96 | (vinu)sUfsauaUfauugauuAfgUfucaggsasa |
| SR462 | (SEQ ID NO. 176) | AWH | (SEQ ID NO. 386) |
| SR214 | cscsugguUfcUfCfAfuuccuugcaa | L96 | (vinu)sUfsgcaAfggaaugaGfaAfccaggsasc |
| SR588 | (SEQ ID NO. 177) | AWH | (SEQ ID NO. 387) |
| SR166 | ususcucaUfuCfCfUfugcaccauaa | L96 | (vinu)sUfsaugGfugcaaggAfaUfgagaascsc |
| SR708 | (SEQ ID NO. 178) | AWH | (SEQ ID NO. 388) |
| SR284 | usasuuggCfuUfUfCfugcuggguua | L96 | (vinu)sAfsaccCfagcagaaAfgCfcaauasasa |
| SR390 | (SEQ ID NO. 179) | AWH | (SEQ ID NO. 389) |
| SR277 | cscsuccuCfuAfCfAfugauuuucca | L96 | (vinu)sGfsgaaAfaucauguAfgAfggaggsgsg |
| SR431 | (SEQ ID NO. 180) | AWH | (SEQ ID NO. 390) |
| SR204 | asgsAfuCfaAfGfGfcucaCfuuaua | L96 | (vinu)sAfsuaaGfuGfAfgccuUfgAfucususg |
| SR540 | (SEQ ID NO. 181) | AWH | (SEQ ID NO. 391) |
| SR117 | uscsAfgCfaUfUfGfugauGfaccaa | L96 | (vinu)sUfsgguCfaUfCfacaaUfgCfugasgsg |
| SR550 | (SEQ ID NO. 182) | AWH | (SEQ ID NO. 392) |
| SR039 | uscsAfuCfaUfUfCfucuuGfugcaa | L96 | (vinu)sUfsgcaCfaAfGfagaaUfgAfugasusc |
| SR396 | (SEQ ID NO. 183) | AWH | (SEQ ID NO. 393) |
| SR152 | usgsGfcCfgUfCfAfcaguUfcucua | L96 | (vinu)sAfsgagAfaCfUfgugaCfgGfccascsa |
| SR609 | (SEQ ID NO. 184) | AWH | (SEQ ID NO. 394) |
| SR288 | usgsGfuCfcCfAfAfagcaAfuucua | L96 | (vinu)sAfsgaaUfuGfCfuuugGfgAfccasgsa |
| SR558 | (SEQ ID NO. 185) | AWH | (SEQ ID NO. 395) |
| SR221 | uscsCfaGfuAfCfAfucuaUfcagaa | L96 | (vinu)sUfscugAfuAfGfauguAfcUfggasasa |
| SR628 | (SEQ ID NO. 186) | AWH | (SEQ ID NO. 396) |
| SR243 | cscsAfgUfaCfAfUfcuauCfagaaa | L96 | (vinu)sUfsucuGfaUfAfgaugUfaCfuggsasa |
| SR620 | (SEQ ID NO. 187) | AWH | (SEQ ID NO. 397) |
| SR145 | asusCfuAfuCfAfGfaaucAfucaaa | L96 | (vinu)sUfsugaUfgAfUfucugAfuAfgausgsu |
| SR482 | (SEQ ID NO. 188) | AWH | (SEQ ID NO. 398) |
| SR217 | asasGfuCfaCfUfCfaauuCfcugaa | L96 | (vinu)sUfscagGfaAfUfugagUfgAfcuususa |
| SR365 | (SEQ ID NO. 189) | AWH | (SEQ ID NO. 399) |
| SR022 | cscsUfgAfaCfUfAfaucaAfuauaa | L96 | (vinu)sUfsauaUfuGfAfuuagUfuCfaggsasa |
| SR395 | (SEQ ID NO. 190) | AWH | (SEQ ID NO. 400) |
| SR008 | usgsAfaCfuAfAfUfcaauAfuauaa | L96 | (vinu)sUfsauaUfaUfUfgauuAfgUfucasgsg |
| SR602 | (SEQ ID NO. 191) | AWH | (SEQ ID NO. 401) |
| SR196 | usgsGfuUfcUfCfAfuuccUfugcaa | L96 | (vinu)sUfsgcaAfgGfAfaugaGfaAfccasgsg |
| SR650 | (SEQ ID NO. 192) | AWH | (SEQ ID NO. 402) |
| SR121 | csusCfaUfuCfCfUfugcaCfcauaa | L96 | (vinu)sUfsaugGfuGfCfaaggAfaUfgagsasa |
| SR520 | (SEQ ID NO. 193) | AWH | (SEQ ID NO. 403) |
| SR310 | ususGfgCfuUfUfCfugcuGfgguua | L96 | (vinu)sAfsaccCfaGfCfagaaAfgCfcaasusa |
| SR699 | (SEQ ID NO. 194) | AWH | (SEQ ID NO. 404) |
| SR139 | uscsCfuCfuAfCfAfugauUfuucca | L96 | (vinu)sGfsgaaAfaUfCfauguAfgAfggasgsg |
| SR533 | (SEQ ID NO. 195) | AWH | (SEQ ID NO. 405) |
| SR149 | asgsauCfaAfGfGfcucaCfUfuaua | L96 | (vinu)sAfsuaaGfuGfAfgccuUfgAfucususg |
| SR464 | (SEQ ID NO. 196) | AWH | (SEQ ID NO. 406) |
| SR207 | uscsagCfaUfUfGfugauGfAfccaa | L96 | (vinu)sUfsgguCfaUfCfacaaUfgCfugasgsg |
| SR433 | (SEQ ID NO. 197) | AWH | (SEQ ID NO. 407) |
| SR226 | uscsauCfaUfUfCfucuuGfUfgcaa | L96 | (vinu)sUfsgcaCfaAfGfagaaUfgAfugasusc |
| SR657 | (SEQ ID NO. 198) | AWH | (SEQ ID NO. 408) |
| SR136 | usgsgcCfgUfCfAfcaguUfCfucua | L96 | (vinu)sAfsgagAfaCfUfgugaCfgGfccascsa |
| SR354 | (SEQ ID NO. 199) | AWH | (SEQ ID NO. 409) |
| SR060 | usgsguCfcCfAfAfagcaAfUfucua | L96 | (vinu)sAfsgaaUfuGfCfuuugGfgAfccasgsa |
| SR399 | (SEQ ID NO. 200) | AWH | (SEQ ID NO. 410) |
| SR307 | uscscaGfuAfCfAfucuaUfCfagaa | L96 | (vinu)sUfscugAfuAfGfauguAfcUfggasasa |
| SR467 | (SEQ ID NO. 201) | AWH | (SEQ ID NO. 411) |
| SR311 | cscsagUfaCfAfUfcuauCfAfgaaa | L96 | (vinu)sUfsucuGfaUfAfgaugUfaCfuggsasa |
| SR440 | (SEQ ID NO. 202) | AWH | (SEQ ID NO. 412) |
| SR268 | asuscuAfuCfAfGfaaucAfUfcaaa | L96 | (vinu)sUfsugaUfgAfUfucugAfuAfgausgsu |
| SR623 | (SEQ ID NO. 203) | AWH | (SEQ ID NO. 413) |
| SR061 | asasguCfaCfUfCfaauuCfCfugaa | L96 | (vinu)sUfscagGfaAfUfugagUfgAfcuususa |
| SR692 | (SEQ ID NO. 204) | AWH | (SEQ ID NO. 414) |
| SR080 | cscsugAfaCfUfAfaucaAfUfauaa | L96 | (vinu)sUfsauaUfuGfAfuuagUfuCfaggsasa |
| SR598 | (SEQ ID NO. 205) | AWH | (SEQ ID NO. 415) |
| SR081 | usgsaaCfuAfAfUfcaauAfUfauaa | L96 | (vinu)sUfsauaUfaUfUfgauuAfgUfucasgsg |
| SR557 | (SEQ ID NO. 206) | AWH | (SEQ ID NO. 416) |
| SR065 | usgsguUfcUfCfAfuuccUfUfgcaa | L96 | (vinu)sUfsgcaAfgGfAfaugaGfaAfccasgsg |
| SR645 | (SEQ ID NO. 207) | AWH | (SEQ ID NO. 417) |
| SR309 | csuscaUfuCfCfUfugcaCfCfauaa | L96 | (vinu)sUfsaugGfuGfCfaaggAfaUfgagsasa |
| SR377 | (SEQ ID NO. 208) | AWH | (SEQ ID NO. 418) |
| SR179 | ususggCfuUfUfCfugcuGfGfguua | L96 | (vinu)sAfsaccCfaGfCfagaaAfgCfcaasusa |
| SR426 | (SEQ ID NO. 209) | AWH | (SEQ ID NO. 419) |
| SR270 | uscscuCfuAfCfAfugauUfUfucca | L96 | (vinu)sGfsgaaAfaUfCfauguAfgAfggasgsg |
| SR391 | (SEQ ID NO. 210) | AWH | (SEQ ID NO. 420) |
| SR241 | asgsauCfaAfGfGfcucaCfUfuaua | L96 | (vinu)sAfsuaaGfugagccuUfgAfucususg |
| SR515 | (SEQ ID NO. 211) | AWH | (SEQ ID NO. 421) |
| SR337 | uscsagCfaUfUfGfugauGfAfccaa | L96 | (vinu)sUfsgguCfaucacaaUfgCfugasgsg |
| SR583 | (SEQ ID NO. 212) | AWH | (SEQ ID NO. 422) |
| SR247 | uscsauCfaUfUfCfucuuGfUfgcaa | L96 | (vinu)sUfsgcaCfaagagaaUfgAfugasusc |
| SR452 | (SEQ ID NO. 213) | AWH | (SEQ ID NO. 423) |
| SR154 | usgsgcCfgUfCfAfcaguUfCfucua | L96 | (vinu)sAfsgagAfacugugaCfgGfccascsa |
| SR346 | (SEQ ID NO. 214) | AWH | (SEQ ID NO. 424) |
| SR103 | usgsguCfcCfAfAfagcaAfUfucua | L96 | (vinu)sAfsgaaUfugcuuugGfgAfccasgsa |
| SR456 | (SEQ ID NO. 215) | AWH | (SEQ ID NO. 425) |
| SR315 | uscscaGfuAfCfAfucuaUfCfagaa | L96 | (vinu)sUfscugAfuagauguAfcUfggasasa |
| SR349 | (SEQ ID NO. 216) | AWH | (SEQ ID NO. 426) |
| SR094 | cscsagUfaCfAfUfcuauCfAfgaaa | L96 | (vinu)sUfsucuGfauagaugUfaCfuggsasa |
| SR493 | (SEQ ID NO. 217) | AWH | (SEQ ID NO. 427) |
| SR336 | asuscuAfuCfAfGfaaucAfUfcaaa | L96 | (vinu)sUfsugaUfgauucugAfuAfgausgsu |
| SR459 | (SEQ ID NO. 218) | AWH | (SEQ ID NO. 428) |
| SR203 | asasguCfaCfUfCfaauuCfCfugaa | L96 | (vinu)sUfscagGfaauugagUfgAfcuususa |
| SR546 | (SEQ ID NO. 219) | AWH | (SEQ ID NO. 429) |
| SR306 | cscsugAfaCfUfAfaucaAfUfauaa | L96 | (vinu)sUfsauaUfugauuagUfuCfaggsasa |
| SR551 | (SEQ ID NO. 220) | AWH | (SEQ ID NO. 430) |
| SR092 | usgsaaCfuAfAfUfcaauAfUfauaa | L96 | (vinu)sUfsauaUfauugauuAfgUfucasgsg |
| SR385 | (SEQ ID NO. 221) | AWH | (SEQ ID NO. 431) |
| SR113 | usgsguUfcUfCfAfuuccUfUfgcaa | L96 | (vinu)sUfsgcaAfggaaugaGfaAfccasgsg |
| SR547 | (SEQ ID NO. 222) | AWH | (SEQ ID NO. 432) |
| SR335 | csuscaUfuCfCfUfugcaCfCfauaa | L96 | (vinu)sUfsaugGfugcaaggAfaUfgagsasa |
| SR443 | (SEQ ID NO. 223) | AWH | (SEQ ID NO. 433) |
| SR001 | ususggCfuUfUfCfugcuGfGfguua | L96 | (vinu)sAfsaccCfagcagaaAfgCfcaasusa |
| SR693 | (SEQ ID NO. 224) | AWH | (SEQ ID NO. 434) |
| SR265 | uscscuCfuAfCfAfugauUfUfucca | L96 | (vinu)sGfsgaaAfaucauguAfgAfggasgsg |
| SR627 | (SEQ ID NO. 225) | AWH | (SEQ ID NO. 435) |
| SR010 | csasagAfuCfaAfGfGfcucaCfuuaua | L96 | (vinu)sAfsuaaGfuGfAfgccuUfgAfucuugscsu |
| SR589 | (SEQ ID NO. 226) | AWH | (SEQ ID NO. 436) |
| SR340 | cscsucAfgCfaUfUfGfugauGfaccaa | L96 | (vinu)sUfsgguCfaUfCfacaaUfgCfugaggsusu |
| SR524 | (SEQ ID NO. 227) | AWH | (SEQ ID NO. 437) |
| SR054 | gsasucAfuCfaUfUfCfucuuGfugcaa | L96 | (vinu)sUfsgcaCfaAfGfagaaUfgAfugaucsasu |
| SR669 | (SEQ ID NO. 228) | AWH | (SEQ ID NO. 438) |
| SR262 | usgsugGfcCfgUfCfAfcaguUfcucu | L96 | (vinu)sAfsgagAfaCfUfgugaCfgGfccacascsu |
| SR491 | a | AWH | (SEQ ID NO. 439) |
| | (SEQ ID NO. 229) | | |
| SR328 | uscsugGfuCfcCfAfAfagcaAfuucua | L96 | (vinu)sAfsgaaUfuGfCfuuugGfgAfccagasasu |
| SR428 | (SEQ ID NO. 230) | AWH | (SEQ ID NO. 440) |
| SR164 | ususucCfaGfuAfCfAfucuaUfcagaa | L96 | (vinu)sUfscugAfuAfGfauguAfcUfggaaasusg |
| SR436 | (SEQ ID NO. 231) | AWH | (SEQ ID NO. 441) |
| SR144 | ususccAfgUfaCfAfUfcuauCfagaaa | L96 | (vinu)sUfsucuGfaUfAfgaugUfaCfuggaasasu |
| SR621 | (SEQ ID NO. 232) | AWH | (SEQ ID NO. 442) |
| SR015 | ascsauCfuAfuCfAfGfaaucAfucaaa | L96 | (vinu)sUfsugaUfgAfUfucugAfuAfgaugusasc |
| SR652 | (SEQ ID NO. 233) | AWH | (SEQ ID NO. 443) |
| SR210 | usasaaGfuCfaCfUfCfaauuCfcugaa | L96 | (vinu)sUfscagGfaAfUfugagUfgAfcuuuasasg |
| SR578 | (SEQ ID NO. 234) | AWH | (SEQ ID NO. 444) |
| SR004 | ususccUfgAfaCfUfAfaucaAfuauaa | L96 | (vinu)sUfsauaUfuGfAfuuagUfuCfaggaasusu |
| SR398 | (SEQ ID NO. 235) | AWH | (SEQ ID NO. 445) |
| SR173 | cscsugAfaCfuAfAfUfcaauAfuauaa | L96 | (vinu)sUfsauaUfaUfUfgauuAfgUfucaggsasa |
| SR501 | (SEQ ID NO. 236) | AWH | (SEQ ID NO. 446) |
| SR330 | cscsugGfuUfcUfCfAfuuccUfugcaa | L96 | (vinu)sUfsgcaAfgGfAfaugaGfaAfccaggsasc |
| SR383 | (SEQ ID NO. 237) | AWH | (SEQ ID NO. 447) |
| SR219 | ususcuCfaUfuCfCfUfugcaCfcauaa | L96 | (vinu)sUfsaugGfuGfCfaaggAfaUfgagaascsc |
| SR635 | (SEQ ID NO. 238) | AWH | (SEQ ID NO. 448) |
| SR007 | usasuuGfgCfuUfUfCfugcuGfgguu | L96 | (vinu)sAfsaccCfaGfCfagaaAfgCfcaauasasa |
| SR535 | a | AWH | (SEQ ID NO. 449) |
| | (SEQ ID NO. 239) | | |
| SR201 | cscsucCfuCfuAfCfAfugauUfuucca | L96 | (vinu)sGfsgaaAfaUfCfauguAfgAfggaggsgsg |
| SR392 | (SEQ ID NO. 240) | AWH | (SEQ ID NO. 450) |
| SR189 | csasagauCfaAfGfGfcucaCfUfuaua | L96 | (vinu)sAfsuaaGfuGfAfgccuUfgAfucuugscsu |
| SR582 | (SEQ ID NO. 241) | AWH | (SEQ ID NO. 451) |
| SR216 | cscsucagCfaUfUfGfugauGfAfccaa | L96 | (vinu)sUfsgguCfaUfCfacaaUfgCfugaggsusu |
| SR463 | (SEQ ID NO. 242) | AWH | (SEQ ID NO. 452) |
| SR021 | gsasucauCfaUfUfCfucuuGfUfgcaa | L96 | (vinu)sUfsgcaCfaAfGfagaaUfgAfugaucsasu |
| SR615 | (SEQ ID NO. 243) | AWH | (SEQ ID NO. 453) |
| SR027 | usgsuggcCfgUfCfAfcaguUfCfucu | L96 | (vinu)sAfsgagAfaCfUfgugaCfgGfccacascsu |
| SR544 | a | AWH | (SEQ ID NO. 454) |
| | (SEQ ID NO. 244) | | |
| SR303 | uscsugguCfcCfAfAfagcaAfUfucua | L96 | (vinu)sAfsgaaUfuGfCfuuugGfgAfccagasasu |
| SR453 | (SEQ ID NO. 245) | AWH | (SEQ ID NO. 455) |
| SR014 | ususuccaGfuAfCfAfucuaUfCfagaa | L96 | (vinu)sUfscugAfuAfGfauguAfcUfggaaasusg |
| SR429 | (SEQ ID NO. 246) | AWH | (SEQ ID NO. 456) |
| SR095 | ususccagUfaCfAfUfcuauCfAfgaaa | L96 | (vinu)sUfsucuGfaUfAfgaugUfaCfuggaasasu |
| SR519 | (SEQ ID NO. 247) | AWH | (SEQ ID NO. 457) |
| SR275 | ascsaucuAfuCfAfGfaaucAfUfcaaa | L96 | (vinu)sUfsugaUfgAfUfucugAfuAfgaugusasc |
| SR458 | (SEQ ID NO. 248) | AWH | (SEQ ID NO. 458) |
| SR012 | usasaaguCfaCfUfCfaauuCfCfugaa | L96 | (vinu)sUfscagGfaAfUfugagUfgAfcuuuasasg |
| SR481 | (SEQ ID NO. 249) | AWH | (SEQ ID NO. 459) |
| SR218 | ususccugAfaCfUfAfaucaAfUfauaa | L96 | (vinu)sUfsauaUfuGfAfuuagUfuCfaggaasusu |
| SR434 | (SEQ ID NO. 250) | AWH | (SEQ ID NO. 460) |
| SR137 | cscsugaaCfuAfAfUfcaauAfUfauaa | L96 | (vinu)sUfsauaUfaUfUfgauuAfgUfucaggsasa |
| SR662 | (SEQ ID NO. 251) | AWH | (SEQ ID NO. 461) |
| SR239 | cscsugguUfcUfCfAfuuccUfUfgcaa | L96 | (vinu)sUfsgcaAfgGfAfaugaGfaAfccaggsasc |
| SR446 | (SEQ ID NO. 252) | AWH | (SEQ ID NO. 462) |
| SR327 | ususcucaUfuCfCfUfugcaCfCfauaa | L96 | (vinu)sUfsaugGfuGfCfaaggAfaUfgagaascsc |
| SR618 | (SEQ ID NO. 253) | AWH | (SEQ ID NO. 463) |
| SR209 | usasuuggCfuUfUfCfugcuGfGfguu | L96 | (vinu)sAfsaccCfaGfCfagaaAfgCfcaauasasa |
| SR408 | a | AWH | (SEQ ID NO. 464) |
| | (SEQ ID NO. 254) | | |
| SR029 | cscsuccuCfuAfCfAfugauUfUfucca | L96 | (vinu)sGfsgaaAfaUfCfauguAfgAfggaggsgsg |
| SR685 | (SEQ ID NO. 255) | AWH | (SEQ ID NO. 465) |
| SR072 | csasagauCfaAfGfGfcucaCfUfuaua | L96 | (vinu)sAfsuaaGfugagccuUfgAfucuugscsu |
| SR449 | (SEQ ID NO. 256) | AWH | (SEQ ID NO. 466) |
| SR312 | cscsucagCfaUfUfGfugauGfAfccaa | L96 | (vinu)sUfsgguCfaucacaaUfgCfugaggsusu |
| SR362 | (SEQ ID NO. 257) | AWH | (SEQ ID NO. 467) |
| SR194 | gsasucauCfaUfUfCfucuuGfUfgcaa | L96 | (vinu)sUfsgcaCfaagagaaUfgAfugaucsasu |
| SR668 | (SEQ ID NO. 258) | AWH | (SEQ ID NO. 468) |
| SR258 | usgsuggcCfgUfCfAfcaguUfCfucu | L96 | (vinu)sAfsgagAfacugugaCfgGfccacascsu |
| SR595 | a | AWH | (SEQ ID NO. 469) |
| | (SEQ ID NO. 259) | | |
| SR142 | uscsugguCfcCfAfAfagcaAfUfucua | L96 | (vinu)sAfsgaaUfugcuuugGfgAfccagasasu |
| SR397 | (SEQ ID NO. 260) | AWH | (SEQ ID NO. 470) |
| SR032 | ususuccaGfuAfCfAfucuaUfCfagaa | L96 | (vinu)sUfscugAfuagauguAfcUfggaaasusg |
| SR404 | (SEQ ID NO. 261) | AWH | (SEQ ID NO. 471) |
| SR305 | ususccagUfaCfAfUfcuauCfAfgaaa | L96 | (vinu)sUfsucuGfauagaugUfaCfuggaasasu |
| SR653 | (SEQ ID NO. 262) | AWH | (SEQ ID NO. 472) |
| SR155 | ascsaucuAfuCfAfGfaaucAfUfcaaa | L96 | (vinu)sUfsugaUfgauucugAfuAfgaugusasc |
| SR647 | (SEQ ID NO. 263) | AWH | (SEQ ID NO. 473) |
| SR227 | usasaaguCfaCfUfCfaauuCfCfugaa | L96 | (vinu)sUfscagGfaauugagUfgAfcuuuasasg |
| SR634 | (SEQ ID NO. 264) | AWH | (SEQ ID NO. 474) |
| SR213 | ususccugAfaCfUfAfaucaAfUfauaa | L96 | (vinu)sUfsauaUfugauuagUfuCfaggaasusu |
| SR423 | (SEQ ID NO. 265) | AWH | (SEQ ID NO. 475) |
| SR273 | cscsugaaCfuAfAfUfcaauAfUfauaa | L96 | (vinu)sUfsauaUfauugauuAfgUfucaggsasa |
| SR420 | (SEQ ID NO. 266) | AWH | (SEQ ID NO. 476) |
| SR109 | cscsugguUfcUfCfAfuuccUfUfgcaa | L96 | (vinu)sUfsgcaAfggaaugaGfaAfccaggsasc |
| SR599 | (SEQ ID NO. 267) | AWH | (SEQ ID NO. 477) |
| SR112 | ususcucaUfuCfCfUfugcaCfCfauaa | L96 | (vinu)sUfsaugGfugcaaggAfaUfgagaascsc |
| SR437 | (SEQ ID NO. 268) | AWH | (SEQ ID NO. 478) |
| SR040 | usasuuggCfuUfUfCfugcuGfGfguua | L96 | (vinu)sAfsaccCfagcagaaAfgCfcaauasasa |
| SR607 | (SEQ ID NO. 269) | AWH | (SEQ ID NO. 479) |
| SR114 | cscsuccuCfuAfCfAfugauUfUfucca | L96 | (vinu)sGfsgaaAfaucauguAfgAfggaggsgsg |
| SR611 | (SEQ ID NO. 270) | AWH | (SEQ ID NO. 480) |

*siRNA modifications:
Nf = 2′-fluoro RNA;
n = 2′-OMe-RNA;
s = phosphorothioate;
(vinu) = vinyl-phosphonate 2′-OMe-uracil

In some embodiments, the siRNA molecule described herein comprises one or more conjugates; said one or more conjugate moieties can be the same conjugates or different conjugates.

In some cases, the siRNA molecule described herein comprises at least one GalNAc moiety, including any GalNAc and derivatives thereof that can be recognized by asialoglycoprotein receptors (ASGPR). In some embodiments, the interaction between the hepatocyte specific ASGPR and GalNAc leads to selectively target a GalNAc-conjugated siRNA to hepatocyte.

GalNAc is conjugated to siRNAs either directly or via a linker.

In some cases, the GalNAc moiety is conjugated to the 3′ end of the sense strand of the siRNA. In some embodiments, the GalNAc moiety is conjugated to the 3′ end nucleotide directly, for example, through a phosphodiester bond. In some embodiments, the GalNAc moiety is conjugated to the 3′ end nucleotide via a linker. In some embodiments, the GalNAc moiety is conjugated to the 5′ end nucleotide directly, for example, through a phosphodiester bond. In some embodiments, the GalNAc moiety is conjugated to the 5′ end nucleotide via a linker. In some embodiments, the conjugated siRNA comprises a formula of A-L-B (A is a siRNA; L is a linker and B is a GalNAc moiety or derivative thereof).

In some examples, the siRNA comprises a GalNAc moiety L96, which has the following structure:

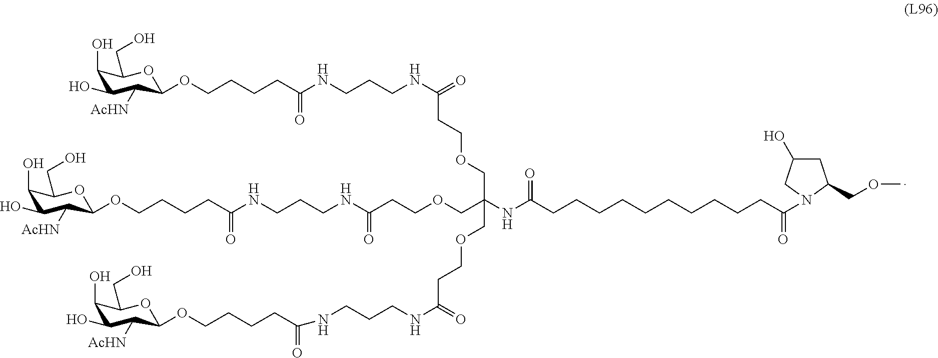

In other examples, the siRNA comprises a GalNAc moiety AWH′, which has the following structure:

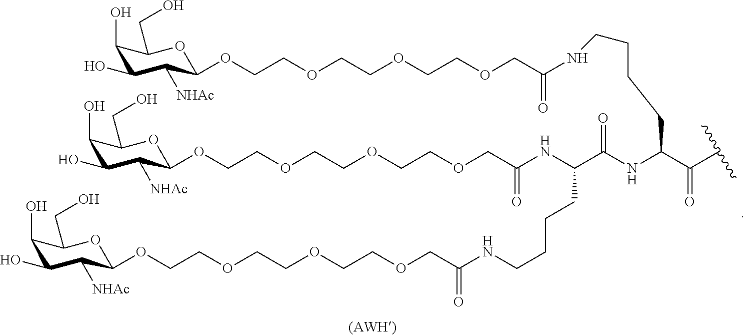

In other examples, the siRNA comprises a GalNAc moiety AWH, which comprises the structure of AWH′ and an aminohexyl linker, and where AWH has the following structure:

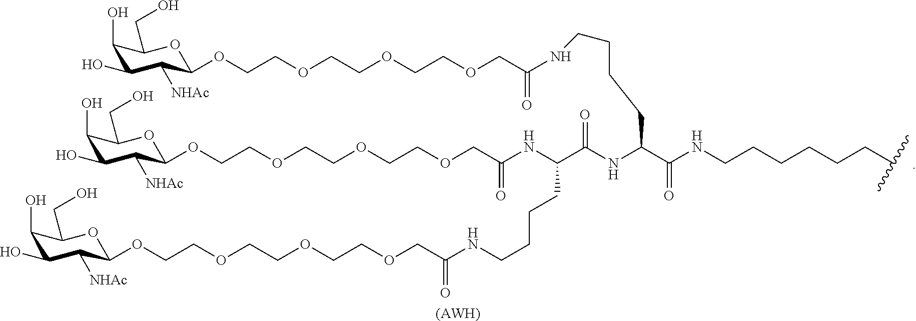

In some embodiments, the GalNAc moiety (e.g., L96 or AWH) is conjugated to the 3′ end of the sense strand of a siRNA molecule described herein. In some examples, the GalNAc moiety (e.g., L96 or AWH) is conjugated to the 3′ end of any one of the modified sense strand of SEQ ID NOs. 61-270 (Table 3). In other examples, the GalNAc moiety (e.g., L96 or AWH) is conjugated to the 3′ end of any one of the sense strand of SEQ ID NOs. 1-15 and 31-45.

In some embodiments, the siRNA molecule targeting a SLC10A1 mRNA transcript does not comprise GalNAc conjugation.

The present disclosure, by bioinformatics selection and knockdown of the expression of SLC10A1 mRNA, further identified additional siRNA molecules that can repress the expression of a human SLC10A1 mRNA transcript specifically and effectively. The siRNAs are listed in Table 4.

In some embodiments, the double-stranded siRNA molecule capable of inhibiting the expression of a human SLC10A1 mRNA transcript comprises a sense strand comprising a sequence at least about 80%, 85%, 90%, 95%, 96%, 97%, 98%, 99%, or 100% identical to a sequence selected from the group consisting of SEQ ID NOs. 481-611 and an antisense strand comprising a sequence at least about 80%, 85%, 90%, 95%, 96%, 97%, 98%, 99%, or 100% identical to the reverse complement of the sense strand of any one of SEQ ID NOs. 481-611. In some cases, the double-stranded siRNA molecule capable of inhibiting the expression of a SLC10A1 mRNA transcript comprises an antisense strand comprising a sequence at least about 80%, 85%, 90%, 95%, 96%, 97%, 98%, 99%, or 100% identical to a sequence selected from SEQ ID NOs. 612-742 and a sense strand comprising a sequence at least about 80%, 85%, 90%, 95%, 96%, 97%, 98%, 99%, or 100% identical to the reverse complement of the antisense strand of any one of SEQ ID NOs. 612-742.

In some embodiments, the siRNA molecule comprises a sense strand and an antisense strand sequences selected from the group consisting of the sequence pairs of SEQ ID NOs. 481 and 612; SEQ ID NOs. 482 and 613; SEQ ID NOs. 483 and 614; SEQ ID NOs. 484 and 615; SEQ ID NOs. 485 and 616; SEQ ID NOs. 486 and 617; SEQ ID NOs. 487 and 618; SEQ ID NOs. 488 and 619; SEQ ID NOs. 489 and 620; SEQ ID NOs. 490 and 621; SEQ ID NOs. 491 and 622; SEQ ID NOs. 492 and 623; SEQ ID NOs. 493 and 624; SEQ ID NOs. 494 and 625; SEQ ID NOs. 495 and 626; SEQ ID NOs. 496 and 627; SEQ ID NOs. 497 and 628; SEQ ID NOs. 498 and 629; SEQ ID NOs. 499 and 630; SEQ ID NOs. 500 and 631; SEQ ID NOs. 501 and 632; SEQ ID NOs. 502 and 633; SEQ ID NOs. 503 and 634; SEQ ID NOs. 504 and 635; SEQ ID NOs. 505 and 636; SEQ ID NOs. 506 and 637; SEQ ID NOs. 507 and 638; SEQ ID NOs. 508 and 639; SEQ ID NOs. 509 and 640; SEQ ID NOs. 510 and 641; SEQ ID NOs. 511 and 642; SEQ ID NOs. 512 and 643; SEQ ID NOs. 513 and 644; SEQ ID NOs. 514 and 645; SEQ ID NOs. 515 and 646; SEQ ID NOs. 516 and 647; SEQ ID NOs. 517 and 648; SEQ ID NOs. 518 and 649; SEQ ID NOs. 519 and 650; SEQ ID NOs. 520 and 651; SEQ ID NOs. 521 and 652; SEQ ID NOs. 522 and 653; SEQ ID NOs. 523 and 654; SEQ ID NOs. 524 and 655; SEQ ID NOs. 525 and 656; SEQ ID NOs. 526 and 657; SEQ ID NOs. 527 and 658; SEQ ID NOs. 528 and 659; SEQ ID NOs. 529 and 660; SEQ ID NOs. 530 and 661; SEQ ID NOs. 531 and 662; SEQ ID NOs. 532 and 663; SEQ ID NOs. 533 and 664; SEQ ID NOs. 534 and 665; SEQ ID NOs. 535 and 666; SEQ ID NOs. 536 and 667; SEQ ID NOs. 537 and 668; SEQ ID NOs. 538 and 669; SEQ ID NOs. 539 and 670; SEQ ID NOs. 540 and 671; SEQ ID NOs. 541 and 672; SEQ ID NOs. 542 and 673; SEQ ID NOs. 543 and 674; SEQ ID NOs. 544 and 675; SEQ ID NOs. 545 and 676; SEQ ID NOs. 546 and 677; SEQ ID NOs. 547 and 678; SEQ ID NOs. 548 and 679; SEQ ID NOs. 549 and 680; SEQ ID NOs. 550 and 681; SEQ ID NOs. 551 and 682; SEQ ID NOs. 552 and 683; SEQ ID NOs. 553 and 684; SEQ ID NOs. 554 and 685; SEQ ID NOs. 555 and 686; SEQ ID NOs. 556 and 687; SEQ ID NOs. 557 and 688; SEQ ID NOs. 558 and 689; SEQ ID NOs. 559 and 690; SEQ ID NOs. 560 and 691; SEQ ID NOs. 561 and 692; SEQ ID NOs. 562 and 693; SEQ ID NOs. 563 and 694; SEQ ID NOs. 564 and 695; SEQ ID NOs. 565 and 696; SEQ ID NOs. 566 and 697; SEQ ID NOs. 567 and 698; SEQ ID NOs. 568 and 699; SEQ ID NOs. 569 and 700; SEQ ID NOs. 570 and 701; SEQ ID NOs. 571 and 702; SEQ ID NOs. 572 and 703; SEQ ID NOs. 573 and 704; SEQ ID NOs. 574 and 705; SEQ ID NOs. 575 and 706; SEQ ID NOs. 576 and 707; SEQ ID NOs. 577 and 708; SEQ ID NOs. 578 and 709; SEQ ID NOs. 579 and 710; SEQ ID NOs. 580 and 711; SEQ ID NOs. 581 and 712; SEQ ID NOs. 582 and 713; SEQ ID NOs. 583 and 714; SEQ ID NOs. 584 and 715; SEQ ID NOs. 585 and 716; SEQ ID NOs. 586 and 717; SEQ ID NOs. 587 and 718; SEQ ID NOs. 588 and 719; SEQ ID NOs. 589 and 720; SEQ ID NOs. 590 and 721; SEQ ID NOs. 591 and 722; SEQ ID NOs. 592 and 723; SEQ ID NOs. 593 and 724; SEQ ID NOs. 594 and 725; SEQ ID NOs. 595 and 726; SEQ ID NOs. 596 and 727; SEQ ID NOs. 597 and 728; SEQ ID NOs. 598 and 729; SEQ ID NOs. 599 and 730; SEQ ID NOs. 600 and 731; SEQ ID NOs. 601 and 732; SEQ ID NOs. 602 and 733; SEQ ID NOs. 603 and 734; SEQ ID NOs. 604 and 735; SEQ ID NOs. 605 and 736; SEQ ID NOs. 606 and 737; SEQ ID NOs. 607 and 738; SEQ ID NOs. 608 and 739; SEQ ID NOs. 609 and 740; SEQ ID NOs. 610 and 741; and SEQ ID NOs. 611 and 742.

TABLE 4
Additional exemplary siRNA molecules targeting
SLC10A1 mRNA

| Duplex | Sense strand | Antisense strand |
|---|---|---|
| ID | sequence (5′-3′) | sequence (5′-3′) |
| SR422 | UGCGCUAUGUCAUCAAGGG | CCCUUGAUGACAUAGCGCAUG |
| | (SEQ ID NO. 481) | (SEQ ID NO. 612) |
| SR479 | UGUCCAAUGUCUUCAGUCU | AGACUGAAGACAUUGGACAGG |
| | (SEQ ID NO. 482) | (SEQ ID NO. 613) |
| SR695 | GAUCAAGGCUCACUUAUGG | CCAUAAGUGAGCCUUGAUCUU |
| | (SEQ ID NO. 483) | (SEQ ID NO. 614) |
| SR425 | AUGUCUUCAGUCUGGCCAU | AUGGCCAGACUGAAGACAUUG |
| | (SEQ ID NO. 484) | (SEQ ID NO. 615) |
| SR466 | CUUGCACCAUAGGGAUCGU | ACGAUCCCUAUGGUGCAAGGA |
| | (SEQ ID NO. 485) | (SEQ ID NO. 616) |
| SR468 | AGUCAUUGGACCACUUUUC | GAAAAGUGGUCCAAUGACUUC |
| | (SEQ ID NO. 486) | (SEQ ID NO. 617) |
| SR460 | CUGAAGUCAUUGGACCACU | AGUGGUCCAAUGACUUCAGGU |
| | (SEQ ID NO. 487) | (SEQ ID NO. 618) |
| SR495 | AGCAUUUCCAGUACAUCUA | UAGAUGUACUGGAAAUGCUGG |
| | (SEQ ID NO. 488) | (SEQ ID NO. 619) |
| SR357 | AAGGGAGGGAUGAUCAUCA | UGAUGAUCAUCCCUCCCUUGA |
| | (SEQ ID NO. 489) | (SEQ ID NO. 620) |
| SR382 | AAUUCCUGAACUAAUCAAU | AUUGAUUAGUUCAGGAAUUGA |
| | (SEQ ID NO. 490) | (SEQ ID NO. 621) |
| SR494 | GAACCUCAGCAUUGUGAUG | CAUCACAAUGCUGAGGUUCAU |
| | (SEQ ID NO. 491) | (SEQ ID NO. 622) |
| SR389 | ACUCAAUUCCUGAACUAAU | AUUAGUUCAGGAAUUGAGUGA |
| | (SEQ ID NO. 492) | (SEQ ID NO. 623) |
| SR644 | UCAUGUUUGCCAUGACACC | GGUGUCAUGGCAAACAUGAUG |
| | (SEQ ID NO. 493) | (SEQ ID NO. 624) |
| SR712 | UAGGGAUCGUCCUCAAAUC | GAUUUGAGGACGAUCCCUAUG |
| | (SEQ ID NO. 494) | (SEQ ID NO. 625) |
| SR418 | GGAGGGAUGAUCAUCAUUC | GAAUGAUGAUCAUCCCUCCCU |
| | (SEQ ID NO. 495) | (SEQ ID NO. 626) |
| SR665 | CAUGCGCUAUGUCAUCAAG | CUUGAUGACAUAGCGCAUGUA |
| | (SEQ ID NO. 496) | (SEQ ID NO. 627) |
| SR667 | CACUCAAUUCCUGAACUAA | UUAGUUCAGGAAUUGAGUGAC |
| | (SEQ ID NO. 497) | (SEQ ID NO. 628) |
| SR704 | UGGUGGCACAGUAUGGCAU | AUGCCAUACUGUGCCACCAGG |
| | (SEQ ID NO. 498) | (SEQ ID NO. 629) |
| SR511 | UGAUGCCUUUUAUUGGCUU | AAGCCAAUAAAAGGCAUCAGG |
| | (SEQ ID NO. 499) | (SEQ ID NO. 630) |
| SR706 | CCCCUCCUCUACAUGAUUU | AAAUCAUGUAGAGGAGGGGAA |
| | (SEQ ID NO. 500) | (SEQ ID NO. 631) |
| SR604 | AGUGGUCUUCCACUGCCUC | GAGGCAGUGGAAGACCACUCC |
| | (SEQ ID NO. 501) | (SEQ ID NO. 632) |
| SR536 | CAAGCGCCCCACAGACCUG | CAGGUCUGUGGGGCGCUUGCC |
| | (SEQ ID NO. 502) | (SEQ ID NO. 633) |
| SR594 | UUCUCAUUCCUUGCACCAU | AUGGUGCAAGGAAUGAGAACC |
| | (SEQ ID NO. 503) | (SEQ ID NO. 634) |
| SR675 | AUUUCCAGUACAUCUAUCA | UGAUAGAUGUACUGGAAAUGC |
| | (SEQ ID NO. 504) | (SEQ ID NO. 635) |
| SR619 | CAUGUUUGCCAUGACACCA | UGGUGUCAUGGCAAACAUGAU |
| | (SEQ ID NO. 505) | (SEQ ID NO. 636) |
| SR587 | AAAUCCCAUUCCAAGUUUU | AAAACUUGGAAUGGGAUUUGG |
| | (SEQ ID NO. 506) | (SEQ ID NO. 637) |
| SR592 | CAUGAACCUCAGCAUUGUG | CACAAUGCUGAGGUUCAUGUC |
| | (SEQ ID NO. 507) | (SEQ ID NO. 638) |
| SR348 | CCACAAUACAUGCGCUAUG | CAUAGCGCAUGUAUUGUGGCC |
| | (SEQ ID NO. 508) | (SEQ ID NO. 639) |
| SR372 | UCACUCAAUUCCUGAACUA | UAGUUCAGGAAUUGAGUGACU |
| | (SEQ ID NO. 509) | (SEQ ID NO. 640) |
| SR393 | GGUGGCACAGUAUGGCAUC | GAUGCCAUACUGUGCCACCAG |
| | (SEQ ID NO. 510) | (SEQ ID NO. 641) |
| SR674 | UGGCUUUCUGCUGGGUUAU | AUAACCCAGCAGAAAGCCAAU |
| | (SEQ ID NO. 511) | (SEQ ID NO. 642) |
| SR406 | AAAUACCCAAAUCCCAUUC | GAAUGGGAUUUGGGUAUUUGA |
| | (SEQ ID NO. 512) | (SEQ ID NO. 643) |
| SR617 | CUUCAUCAUGCUCUCGCUG | CAGCGAGAGCAUGAUGAAGAA |
| | (SEQ ID NO. 513) | (SEQ ID NO. 644) |
| SR606 | AUAUAUAUGUUUAACCCAU | AUGGGUUAAACAUAUAUAUUG |
| | (SEQ ID NO. 514) | (SEQ ID NO. 645) |
| SR489 | ACUGCCUCACAGGAGGAUG | CAUCCUCCUGUGAGGCAGUGG |
| | (SEQ ID NO. 515) | (SEQ ID NO. 646) |
| SR664 | UCAAAUCCAAACGGCCACA | UGUGGCCGUUUGGAUUUGAGG |
| | (SEQ ID NO. 516) | (SEQ ID NO. 647) |
| SR663 | UCUACAUGAUUUUCCAGCU | AGCUGGAAAAUCAUGUAGAGG |
| | (SEQ ID NO. 517) | (SEQ ID NO. 648) |
| SR500 | AGACUCCCAAGGAUAAAAC | GUUUUAUCCUUGGGAGUCUUG |
| | (SEQ ID NO. 518) | (SEQ ID NO. 649) |
| SR523 | CACCCAACUUUGGCAAGCG | CGCUUGCCAAAGUUGGGUGGC |
| | (SEQ ID NO. 519) | (SEQ ID NO. 650) |
| SR658 | UCCUCAAAUCCAAACGGCC | GGCCGUUUGGAUUUGAGGACG |
| | (SEQ ID NO. 520) | (SEQ ID NO. 651) |
| SR656 | CUUGUGCAGUGUGGCCGUC | GACGGCCACACUGCACAAGAG |
| | (SEQ ID NO. 521) | (SEQ ID NO. 652) |
| SR566 | AGAACUGCACAAGAAACGG | CCGUUUCUUGUGCAGUUCUUG |
| | (SEQ ID NO. 522) | (SEQ ID NO. 653) |
| SR517 | UGCACAAGAAACGGAGUCA | UGACUCCGUUUCUUGUGCAGU |
| | (SEQ ID NO. 523) | (SEQ ID NO. 654) |
| SR444 | ACCUGAAGUCAUUGGACCA | UGGUCCAAUGACUUCAGGUGG |
| | (SEQ ID NO. 524) | (SEQ ID NO. 655) |
| SR680 | GCCACCCAACUUUGGCAAG | CUUGCCAAAGUUGGGUGGCAG |
| | (SEQ ID NO. 525) | (SEQ ID NO. 656) |
| SR522 | GCACCAUAGGGAUCGUCCU | AGGACGAUCCCUAUGGUGCAA |
| | (SEQ ID NO. 526) | (SEQ ID NO. 657) |
| SR379 | CAGUGUGGCCGUCACAGUU | AACUGUGACGGCCACACUGCA |
| | (SEQ ID NO. 527) | (SEQ ID NO. 658) |
| SR386 | CAAACGGCCACAAUACAUG | CAUGUAUUGUGGCCGUUUGGA |
| | (SEQ ID NO. 528) | (SEQ ID NO. 659) |
| SR672 | UGCUGGGUUAUGUUCUCUC | GAGAGAACAUAACCCAGCAGA |
| | (SEQ ID NO. 529) | (SEQ ID NO. 660) |
| SR636 | CUCACUUAUGGAAGCCUAA | UUAGGCUUCCAUAAGUGAGCC |
| | (SEQ ID NO. 530) | (SEQ ID NO. 661) |
| SR561 | GGCCGUCACAGUUCUCUCU | AGAGAGAACUGUGACGGCCAC |
| | (SEQ ID NO. 531) | (SEQ ID NO. 662) |
| SR694 | UGCAGUGUGGCCGUCACAG | CUGUGACGGCCACACUGCACA |
| | (SEQ ID NO. 532) | (SEQ ID NO. 663) |
| SR553 | UCAUGCUCUCGCUGGGCUG | CAGCCCAGCGAGAGCAUGAUG |
| | (SEQ ID NO. 533) | (SEQ ID NO. 664) |
| SR478 | AUAGGGAUCGUCCUCAAAU | AUUUGAGGACGAUCCCUAUGG |
| | (SEQ ID NO. 534) | (SEQ ID NO. 665) |
| SR490 | UUCCAAGUUUUGUGACCCA | UGGGUCACAAAACUUGGAAUG |
| | (SEQ ID NO. 535) | (SEQ ID NO. 666) |
| SR485 | CAGCAUUGUGAUGACCACC | GGUGGUCAUCACAAUGCUGAG |
| | (SEQ ID NO. 536) | (SEQ ID NO. 667) |
| SR469 | GCAGUGUGGCCGUCACAGU | ACUGUGACGGCCACACUGCAC |
| | (SEQ ID NO. 537) | (SEQ ID NO. 668) |
| SR381 | CAUGAUUUUCCAGCUUGGA | UCCAAGCUGGAAAAUCAUGUA |
| | (SEQ ID NO. 538) | (SEQ ID NO. 669) |
| SR705 | GCUCACUUAUGGAAGCCUA | UAGGCUUCCAUAAGUGAGCCU |
| | (SEQ ID NO. 539) | (SEQ ID NO. 670) |
| SR673 | AUCCAAACGGCCACAAUAC | GUAUUGUGGCCGUUUGGAUUU |
| | (SEQ ID NO. 540) | (SEQ ID NO. 671) |
| SR486 | AUCAAGGGAGGGAUGAUCA | UGAUCAUCCCUCCCUUGAUGA |
| | (SEQ ID NO. 541) | (SEQ ID NO. 672) |
| SR514 | CGGCCACAAUACAUGCGCU | AGCGCAUGUAUUGUGGCCGUU |
| | (SEQ ID NO. 542) | (SEQ ID NO. 673) |
| SR542 | GGAUGGAGGCCCACAACGC | GCGUUGUGGGCCUCCAUCCUC |
| | (SEQ ID NO. 543) | (SEQ ID NO. 674) |
| SR660 | CGCCCUGGUGGCACAGUAU | AUACUGUGCCACCAGGGCGAU |
| | (SEQ ID NO. 544) | (SEQ ID NO. 675) |
| SR405 | GCAAGCGCCCCACAGACCU | AGGUCUGUGGGGCGCUUGCCA |
| | (SEQ ID NO. 545) | (SEQ ID NO. 676) |
| SR394 | UGGCCGGGAACACAGACAG | CUGUCUGUGUUCCCGGCCAAG |
| | (SEQ ID NO. 546) | (SEQ ID NO. 677) |
| SR560 | AGGGAGGGAUGAUCAUCAU | AUGAUGAUCAUCCCUCCCUUG |
| | (SEQ ID NO. 547) | (SEQ ID NO. 678) |
| SR701 | CCAACUUUGGCAAGCGCCC | GGGCGCUUGCCAAAGUUGGGU |
| | (SEQ ID NO. 548) | (SEQ ID NO. 679) |
| SR626 | ACAGUAUGGCAUCAUGCCC | GGGCAUGAUGCCAUACUGUGC |
| | (SEQ ID NO. 549) | (SEQ ID NO. 680) |
| SR359 | CUGUCCAAUGUCUUCAGUC | GACUGAAGACAUUGGACAGGU |
| | (SEQ ID NO. 550) | (SEQ ID NO. 681) |
| SR576 | CACCAUAGGGAUCGUCCUC | GAGGACGAUCCCUAUGGUGCA |
| | (SEQ ID NO. 551) | (SEQ ID NO. 682) |
| SR525 | ACCAUAGGGAUCGUCCUCA | UGAGGACGAUCCCUAUGGUGC |
| | (SEQ ID NO. 552) | (SEQ ID NO. 683) |
| SR380 | CGUCCUCAAAUCCAAACGG | CCGUUUGGAUUUGAGGACGAU |
| | (SEQ ID NO. 553) | (SEQ ID NO. 684) |
| SR492 | AGGAUGGAGGCCCACAACG | CGUUGUGGGCCUCCAUCCUCC |
| | (SEQ ID NO. 554) | (SEQ ID NO. 685) |
| SR415 | AUGCGCUAUGUCAUCAAGG | CCUUGAUGACAUAGCGCAUGU |
| | (SEQ ID NO. 555) | (SEQ ID NO. 686) |
| SR638 | AAUACCCAAAUCCCAUUCC | GGAAUGGGAUUUGGGUAUUUG |
| | (SEQ ID NO. 556) | (SEQ ID NO. 687) |
| SR351 | CAUAGGGAUCGUCCUCAAA | UUUGAGGACGAUCCCUAUGGU |
| | (SEQ ID NO. 557) | (SEQ ID NO. 688) |
| SR505 | GCGCUAUGUCAUCAAGGGA | UCCCUUGAUGACAUAGCGCAU |
| | (SEQ ID NO. 558) | (SEQ ID NO. 689) |
| SR435 | GUAUGGCAUCAUGCCCCUC | GAGGGGCAUGAUGCCAUACUG |
| | (SEQ ID NO. 559) | (SEQ ID NO. 690) |
| SR568 | AUUCCUUGCACCAUAGGGA | UCCCUAUGGUGCAAGGAAUGA |
| | (SEQ ID NO. 560) | (SEQ ID NO. 691) |
| SR539 | GGACAAGGUGCCCUAUAAA | UUUAUAGGGCACCUUGUCCUU |
| | (SEQ ID NO. 561) | (SEQ ID NO. 692) |
| SR347 | UGCACCAUAGGGAUCGUCC | GGACGAUCCCUAUGGUGCAAG |
| | (SEQ ID NO. 562) | (SEQ ID NO. 693) |
| SR686 | AGUGUGGCCGUCACAGUUC | GAACUGUGACGGCCACACUGC |
| | (SEQ ID NO. 563) | (SEQ ID NO. 694) |
| SR471 | UCACUUAUGGAAGCCUAAA | UUUAGGCUUCCAUAAGUGAGC |
| | (SEQ ID NO. 564) | (SEQ ID NO. 695) |
| SR498 | GCCCUGGUGGCACAGUAUG | CAUACUGUGCCACCAGGGCGA |
| | (SEQ ID NO. 565) | (SEQ ID NO. 696) |
| SR538 | CAGUAUGGCAUCAUGCCCC | GGGGCAUGAUGCCAUACUGUG |
| | (SEQ ID NO. 566) | (SEQ ID NO. 697) |
| SR648 | UUCUGCUGGGUUAUGUUCU | AGAACAUAACCCAGCAGAAAG |
| | (SEQ ID NO. 567) | (SEQ ID NO. 698) |
| SR488 | CUGCACAAGAAACGGAGUC | GACUCCGUUUCUUGUGCAGUU |
| | (SEQ ID NO. 568) | (SEQ ID NO. 699) |
| SR537 | ACCCUGCCACCCAACUUUG | CAAAGUUGGGUGGCAGGGUGA |
| | (SEQ ID NO. 569) | (SEQ ID NO. 700) |
| SR671 | GCCCUUGGCAUGAUGCCUC | GAGGCAUCAUGCCAAGGGCAC |
| | (SEQ ID NO. 570) | (SEQ ID NO. 701) |
| SR570 | UCCAAACGGCCACAAUACA | UGUAUUGUGGCCGUUUGGAUU |
| | (SEQ ID NO. 571) | (SEQ ID NO. 702) |
| SR502 | CUUCAGUCUGGCCAUGAAG | CUUCAUGGCCAGACUGAAGAC |
| | (SEQ ID NO. 572) | (SEQ ID NO. 703) |
| SR688 | CCUGAAGUCAUUGGACCAC | GUGGUCCAAUGACUUCAGGUG |
| | (SEQ ID NO. 573) | (SEQ ID NO. 704) |
| SR367 | GGAGUGGUCUUCCACUGCC | GGCAGUGGAAGACCACUCCUU |
| | (SEQ ID NO. 574) | (SEQ ID NO. 705) |
| SR472 | AACUUUGGCAAGCGCCCCA | UGGGGCGCUUGCCAAAGUUGG |
| | (SEQ ID NO. 575) | (SEQ ID NO. 706) |
| SR432 | CACUUAUGGAAGCCUAAAG | CUUUAGGCUUCCAUAAGUGAG |
| | (SEQ ID NO. 576) | (SEQ ID NO. 707) |
| SR409 | AGGGAUCGUCCUCAAAUCC | GGAUUUGAGGACGAUCCCUAU |
| | (SEQ ID NO. 577) | (SEQ ID NO. 708) |
| SR400 | GUCCUCAAAUCCAAACGGC | GCCGUUUGGAUUUGAGGACGA |
| | (SEQ ID NO. 578) | (SEQ ID NO. 709) |
| SR691 | AACAAAAAUGAUCUACACA | UGUGUAGAUCAUUUUUGUUUU |
| | (SEQ ID NO. 579) | (SEQ ID NO. 710) |
| SR353 | GGCUGCACCAUGGAGUUCA | UGAACUCCAUGGUGCAGCCCA |
| | (SEQ ID NO. 580) | (SEQ ID NO. 711) |
| SR585 | CCUUGCACCAUAGGGAUCG | CGAUCCCUAUGGUGCAAGGAA |
| | (SEQ ID NO. 581) | (SEQ ID NO. 712) |
| SR593 | CCUGCCACCCAACUUUGGC | GCCAAAGUUGGGUGGCAGGGU |
| | (SEQ ID NO. 582) | (SEQ ID NO. 713) |
| SR416 | CAUUCCUUGCACCAUAGGG | CCCUAUGGUGCAAGGAAUGAG |
| | (SEQ ID NO. 583) | (SEQ ID NO. 714) |
| SR654 | GGCCACAAUACAUGCGCUA | UAGCGCAUGUAUUGUGGCCGU |
| | (SEQ ID NO. 584) | (SEQ ID NO. 715) |
| SR590 | UUGCACAGCCUAGCCCUUC | GAAGGGCUAGGCUGUGCAAGG |
| | (SEQ ID NO. 585) | (SEQ ID NO. 716) |
| SR545 | CAUGCUCUCGCUGGGCUGC | GCAGCCCAGCGAGAGCAUGAU |
| | (SEQ ID NO. 586) | (SEQ ID NO. 717) |
| SR363 | ACCCAACUUUGGCAAGCGC | GCGCUUGCCAAAGUUGGGUGG |
| | (SEQ ID NO. 587) | (SEQ ID NO. 718) |
| SR369 | ACAAGGUGCCCUAUAAAGG | CCUUUAUAGGGCACCUUGUCC |
| | (SEQ ID NO. 588) | (SEQ ID NO. 719) |
| SR411 | AAGGUGCCCUAUAAAGGCA | UGCCUUUAUAGGGCACCUUGU |
| | (SEQ ID NO. 589) | (SEQ ID NO. 720) |
| SR610 | AUCGUCCUCAAAUCCAAAC | GUUUGGAUUUGAGGACGAUCC |
| | (SEQ ID NO. 590) | (SEQ ID NO. 721) |
| SR441 | GGCUCACUUAUGGAAGCCU | AGGCUUCCAUAAGUGAGCCUU |
| | (SEQ ID NO. 591) | (SEQ ID NO. 722) |
| SR711 | AAGAUCAAGGCUCACUUAU | AUAAGUGAGCCUUGAUCUUGC |
| | (SEQ ID NO. 592) | (SEQ ID NO. 723) |
| SR388 | AUCAAGGCUCACUUAUGGA | UCCAUAAGUGAGCCUUGAUCU |
| | (SEQ ID NO. 593) | (SEQ ID NO. 724) |
| SR637 | CUCAGCAUUGUGAUGACCA | UGGUCAUCACAAUGCUGAGGU |
| | (SEQ ID NO. 594) | (SEQ ID NO. 725) |
| SR574 | AGGUGCCCUAUAAAGGCAU | AUGCCUUUAUAGGGCACCUUG |
| | (SEQ ID NO. 595) | (SEQ ID NO. 726) |
| SR586 | UCAUUCCUUGCACCAUAGG | CCUAUGGUGCAAGGAAUGAGA |
| | (SEQ ID NO. 596) | (SEQ ID NO. 727) |
| SR614 | GUCAUCAAGGGAGGGAUGA | UCAUCCCUCCCUUGAUGACAU |
| | (SEQ ID NO. 597) | (SEQ ID NO. 728) |
| SR575 | CAAUACAUGCGCUAUGUCA | UGACAUAGCGCAUGUAUUGUG |
| | (SEQ ID NO. 598) | (SEQ ID NO. 729) |
| SR360 | GACAAGGUGCCCUAUAAAG | CUUUAUAGGGCACCUUGUCCU |
| | (SEQ ID NO. 599) | (SEQ ID NO. 730) |
| SR681 | AAGGAGUGGUCUUCCACUG | CAGUGGAAGACCACUCCUUGU |
| | (SEQ ID NO. 600) | (SEQ ID NO. 731) |
| SR616 | UCCCCUUGCACAGCCUAGC | GCUAGGCUGUGCAAGGGGAGC |
| | (SEQ ID NO. 601) | (SEQ ID NO. 732) |
| SR368 | ACAUGCGCUAUGUCAUCAA | UUGAUGACAUAGCGCAUGUAU |
| | (SEQ ID NO. 602) | (SEQ ID NO. 733) |
| SR659 | AAUAUAUAUGUUUAACCCA | UGGGUUAAACAUAUAUAUUGA |
| | (SEQ ID NO. 603) | (SEQ ID NO. 734) |
| SR531 | UUCAUCAUGCUCUCGCUGG | CCAGCGAGAGCAUGAUGAAGA |
| | (SEQ ID NO. 604) | (SEQ ID NO. 735) |
| SR591 | AUCAUGCUCUCGCUGGGCU | AGCCCAGCGAGAGCAUGAUGA |
| | (SEQ ID NO. 605) | (SEQ ID NO. 736) |
| SR549 | AAGGACAAGGUGCCCUAUA | UAUAGGGCACCUUGUCCUUCA |
| | (SEQ ID NO. 606) | (SEQ ID NO. 737) |
| SR503 | CUCCCCUUGCACAGCCUAG | CUAGGCUGUGCAAGGGGAGCA |
| | (SEQ ID NO. 607) | (SEQ ID NO. 738) |
| SR352 | UUGCACCAUAGGGAUCGUC | GACGAUCCCUAUGGUGCAAGG |
| | (SEQ ID NO. 608) | (SEQ ID NO. 739) |
| SR510 | CAAGGUGCCCUAUAAAGGC | GCCUUUAUAGGGCACCUUGUC |
| | (SEQ ID NO. 609) | (SEQ ID NO. 740) |
| SR548 | GUUCUCAUUCCUUGCACCA | UGGUGCAAGGAAUGAGAACCA |
| | (SEQ ID NO. 610) | (SEQ ID NO. 741) |
| SR577 | CAUCAUGCUCUCGCUGGGC | GCCCAGCGAGAGCAUGAUGAA |
| | (SEQ ID NO. 611) | (SEQ ID NO. 742) |

As discussed above, the siRNA molecule may comprise one or more modifications. In some embodiments, the sense strand of the siRNA in Table 4 may comprise one or more modifications. In some embodiments, the antisense strand of the siRNA in Table 4 may comprise one or more modifications. In some embodiments, the sense and antisense strands of the siRNA duplex in Table 4 may comprise one or more modifications. In some embodiments, the last nucleotide of the sense strand of a siRNA molecule is modified as adenine. In some embodiments, the first nucleotide of the antisense strand of a siRNA molecule is modified as uracil.

The siRNA molecule targeting a SLC10A1 mRNA transcript comprises a pair of a modified sense strand and a modified antisense strand in Table 5. As non-limiting examples, the siRNA molecule comprises a modified sense strand and a modified antisense strand selected from the group consisting of the sequence pairs of SEQ ID NOs. 743 and 874; SEQ ID NOs. 744 and 875; SEQ ID NOs. 745 and 876; SEQ ID NOs. 746 and 877; SEQ ID NOs. 747 and 878; SEQ ID NOs. 748 and 879; SEQ ID NOs. 749 and 880; SEQ ID NOs. 750 and 881; SEQ ID NOs. 751 and 882; SEQ ID NOs. 752 and 883; SEQ ID NOs. 753 and 884; SEQ ID NOs. 754 and 885; SEQ ID NOs. 755 and 886; SEQ ID NOs. 756 and 887; SEQ ID NOs. 757 and 888; SEQ ID NOs. 758 and 889; SEQ ID NOs. 759 and 890; SEQ ID NOs. 760 and 891; SEQ ID NOs. 761 and 892; SEQ ID NOs. 762 and 893; SEQ ID NOs. 763 and 894; SEQ ID NOs. 764 and 895; SEQ ID NOs. 765 and 896; SEQ ID NOs. 766 and 897; SEQ ID NOs. 767 and 898; SEQ ID NOs. 768 and 899; SEQ ID NOs. 769 and 900; SEQ ID NOs. 770 and 901; SEQ ID NOs. 771 and 902; SEQ ID NOs. 772 and 903; SEQ ID NOs. 773 and 904; SEQ ID NOs. 774 and 905; SEQ ID NOs. 775 and 906; SEQ ID NOs. 776 and 907; SEQ ID NOs. 777 and 908; SEQ ID NOs. 778 and 909; SEQ ID NOs. 779 and 910; SEQ ID NOs. 780 and 911; SEQ ID NOs. 781 and 912; SEQ ID NOs. 782 and 913; SEQ ID NOs. 783 and 914; SEQ ID NOs. 784 and 915; SEQ ID NOs. 785 and 916; SEQ ID NOs. 786 and 917; SEQ ID NOs. 787 and 918; SEQ ID NOs. 788 and 919; SEQ ID NOs. 789 and 920; SEQ ID NOs. 790 and 921; SEQ ID NOs. 791 and 922; SEQ ID NOs. 792 and 923; SEQ ID NOs. 793 and 924; SEQ ID NOs. 794 and 925; SEQ ID NOs. 795 and 926; SEQ ID NOs. 796 and 927; SEQ ID NOs. 797 and 928; SEQ ID NOs. 798 and 929; SEQ ID NOs. 799 and 930; SEQ ID NOs. 800 and 931; SEQ ID NOs. 801 and 932; SEQ ID NOs. 802 and 933; SEQ ID NOs. 803 and 934; SEQ ID NOs. 804 and 935; SEQ ID NOs. 805 and 936; SEQ ID NOs. 806 and 937; SEQ ID NOs. 807 and 938; SEQ ID NOs. 808 and 939; SEQ ID NOs. 809 and 940; SEQ ID NOs. 810 and 941; SEQ ID NOs. 811 and 942; SEQ ID NOs. 812 and 943; SEQ ID NOs. 813 and 944; SEQ ID NOs. 814 and 945; SEQ ID NOs. 815 and 946; SEQ ID NOs. 816 and 947; SEQ ID NOs. 817 and 948; SEQ ID NOs. 818 and 949; SEQ ID NOs. 819 and 950; SEQ ID NOs. 820 and 951; SEQ ID NOs. 821 and 952; SEQ ID NOs. 822 and 953; SEQ ID NOs. 823 and 954; SEQ ID NOs. 824 and 955; SEQ ID NOs. 825 and 956; SEQ ID NOs. 826 and 957; SEQ ID NOs. 827 and 958; SEQ ID NOs. 828 and 959; SEQ ID NOs. 829 and 960; SEQ ID NOs. 830 and 961; SEQ ID NOs. 831 and 962; SEQ ID NOs. 832 and 963; SEQ ID NOs. 833 and 964; SEQ ID NOs. 834 and 965; SEQ ID NOs. 835 and 966; SEQ ID NOs. 836 and 967; SEQ ID NOs. 837 and 968; SEQ ID NOs. 838 and 969; SEQ ID NOs. 839 and 970; SEQ ID NOs. 840 and 971; SEQ ID NOs. 841 and 972; SEQ ID NOs. 842 and 973; SEQ ID NOs. 843 and 974; SEQ ID NOs. 844 and 975; SEQ ID NOs. 845 and 976; SEQ ID NOs. 846 and 977; SEQ ID NOs. 847 and 978; SEQ ID NOs. 848 and 979; SEQ ID NOs. 849 and 980; SEQ ID NOs. 850 and 981; SEQ ID NOs. 851 and 982; SEQ ID NOs. 852 and 983; SEQ ID NOs. 853 and 984; SEQ ID NOs. 854 and 985; SEQ ID NOs. 855 and 986; SEQ ID NOs. 856 and 987; SEQ ID NOs. 857 and 988; SEQ ID NOs. 858 and 989; SEQ ID NOs. 859 and 990; SEQ ID NOs. 860 and 991; SEQ ID NOs. 861 and 992; SEQ ID NOs. 862 and 993; SEQ ID NOs. 863 and 994; SEQ ID NOs. 864 and 995; SEQ ID NOs. 865 and 996; SEQ ID NOs.866 and 997; SEQ ID NOs. 867 and 998; SEQ ID NOs. 868 and 999; SEQ ID NOs. 869 and 1000; SEQ ID NOs. 870 and 1001; SEQ ID NOs. 871 and 1002; SEQ ID NOs. 872 and 1003; and SEQ ID NOs. 873 and 1004.

In some embodiments, the siRNA molecule further comprises GalNAc at the 3′ end of the sense strand. The GalNAc is L96 (Table 5). In some cases, the GalNAc is AWH. In some embodiments, the siRNA molecule does not comprise GalNAc. In some examples, the GalNAc moiety (e.g., L96 or AWH) is conjugated to the 3′ end of any one of the sense strand of SEQ ID NOs. 481-611. In other examples, the GalNAc moiety (e.g., L96 or AWH) is conjugated to the 3′ end of any one of the modified sense strand of SEQ ID NOs. 743-873.

TABLE 5
Modified sense and antisense strands of siRNAs

| | | GalNAc at | |
|---|---|---|---|
| Duplex | Sense (S) strand sequence | the 3′end of | Antisense strand (AS) sequence |
| ID | (5′-3′) | the S strand | (5′-3′) |
| SR002 | gsusccUfcAfAfAfuccaaacgga | L96 | usCfscguUfuggauuuGfaGfgacsgsa |
| SR827 | (SEQ ID NO. 743) | AWH | (SEQ ID NO. 874) |
| SR003 | gscsacCfaUfAfGfggaucgucca | L96 | usGfsgacGfaucccuaUfgGfugcsasa |
| SR769 | (SEQ ID NO. 744) | AWH | (SEQ ID NO. 875) |
| SR005 | asusucCfuUfGfCfaccauaggga | L96 | usCfsccuAfuggugcaAfgGfaausgsa |
| SR765 | (SEQ ID NO. 745) | AWH | (SEQ ID NO. 876) |
| SR013 | gsgsugGfcAfCfAfguauggcaua | L96 | usAfsugcCfauacuguGfcCfaccsasg |
| SR723 | (SEQ ID NO. 746) | AWH | (SEQ ID NO. 877) |
| SR016 | csusgcAfcAfAfGfaaacggagua | L96 | usAfscucCfguuucuuGfuGfcagsusu |
| SR772 | (SEQ ID NO. 747) | AWH | (SEQ ID NO. 878) |
| SR018 | ususccAfaGfUfUfuugugaccca | L96 | usGfsgguCfacaaaacUfuGfgaasusg |
| SR747 | (SEQ ID NO. 748) | AWH | (SEQ ID NO. 879) |
| SR024 | csasugAfaCfCfUfcagcauugua | L96 | usAfscaaUfgcugaggUfuCfaugsusc |
| SR787 | (SEQ ID NO. 749) | AWH | (SEQ ID NO. 880) |
| SR025 | asasgaUfcAfAfGfgcucacuuaa | L96 | usUfsaagUfgagccuuGfaUfcuusgsc |
| SR837 | (SEQ ID NO. 750) | AWH | (SEQ ID NO. 881) |
| SR026 | ususcuGfcUfGfGfguuauguuca | L96 | usGfsaacAfuaacccaGfcAfgaasasg |
| SR761 | (SEQ ID NO. 751) | AWH | (SEQ ID NO. 882) |
| SR030 | gsasacCfuCfAfGfcauugugaua | L96 | usAfsucaCfaaugcugAfgGfuucsasu |
| SR718 | (SEQ ID NO. 752) | AWH | (SEQ ID NO. 883) |
| SR033 | csascuUfaUfGfGfaagccuaaaa | L96 | usUfsuuaGfgcuuccaUfaAfgugsasg |
| SR763 | (SEQ ID NO. 753) | AWH | (SEQ ID NO. 884) |
| SR034 | csasauAfcAfUfGfcgcuauguca | L96 | usGfsacaUfagcgcauGfuAfuugsusg |
| SR812 | (SEQ ID NO. 754) | AWH | (SEQ ID NO. 885) |
| SR035 | csusucAfuCfAfUfgcucucgcua | L96 | usAfsgcgAfgagcaugAfuGfaagsasa |
| SR771 | (SEQ ID NO. 755) | AWH | (SEQ ID NO. 886) |
| SR036 | asuscaUfgCfUfCfucgcugggca | L96 | usGfscccAfgcgagagCfaUfgausgsa |
| SR742 | (SEQ ID NO. 756) | AWH | (SEQ ID NO. 887) |
| SR037 | asasauCfcCfAfUfuccaaguuua | L96 | usAfsaacUfuggaaugGfgAfuuusgsg |
| SR779 | (SEQ ID NO. 757) | AWH | (SEQ ID NO. 888) |
| SR043 | uscsuaCfaUfGfAfuuuuccagca | L96 | usGfscugGfaaaaucaUfgUfagasgsg |
| SR743 | (SEQ ID NO. 758) | AWH | (SEQ ID NO. 889) |
| SR045 | usgscuGfgGfUfUfauguucucua | L96 | usAfsgagAfacauaacCfcAfgcasgsa |
| SR795 | (SEQ ID NO. 759) | AWH | (SEQ ID NO. 890) |
| SR046 | asgsggAfuCfGfUfccucaaauca | L96 | usGfsauuUfgaggacgAfuCfccusasu |
| SR735 | (SEQ ID NO. 760) | AWH | (SEQ ID NO. 891) |
| SR048 | csasguAfuGfGfCfaucaugccca | L96 | usGfsggcAfugaugccAfuAfcugsusg |
| SR797 | (SEQ ID NO. 761) | AWH | (SEQ ID NO. 892) |
| SR049 | ususcaUfcAfUfGfcucucgcuga | L96 | usCfsagcGfagagcauGfaUfgaasgsa |
| SR817 | (SEQ ID NO. 762) | AWH | (SEQ ID NO. 893) |
| SR050 | ususcuCfaUfUfCfcuugcaccaa | L96 | usUfsgguGfcaaggaaUfgAfgaascsc |
| SR834 | (SEQ ID NO. 763) | AWH | (SEQ ID NO. 894) |
| SR051 | gsgscuGfcAfCfCfauggaguuca | L96 | usGfsaacUfccaugguGfcAfgccscsa |
| SR750 | (SEQ ID NO. 764) | AWH | (SEQ ID NO. 895) |
| SR057 | asascuUfuGfGfCfaagcgcccca | L96 | usGfsgggCfgcuugccAfaAfguusgsg |
| SR815 | (SEQ ID NO. 765) | AWH | (SEQ ID NO. 896) |
| SR059 | asgsucAfuUfGfGfaccacuuuua | L96 | usAfsaaaGfugguccaAfuGfacususc |
| SR782 | (SEQ ID NO. 766) | AWH | (SEQ ID NO. 897) |
| SR062 | usgsguGfgCfAfCfaguauggcaa | L96 | usUfsgccAfuacugugCfcAfccasgsg |
| SR828 | (SEQ ID NO. 767) | AWH | (SEQ ID NO. 898) |
| SR064 | asgsggAfgGfGfAfugaucaucaa | L96 | usUfsgauGfaucauccCfuCfccususg |
| SR783 | (SEQ ID NO. 768) | AWH | (SEQ ID NO. 899) |
| SR067 | cscsugAfaGfUfCfauuggaccaa | L96 | usUfsgguCfcaaugacUfuCfaggsusg |
| SR767 | (SEQ ID NO. 769) | AWH | (SEQ ID NO. 900) |
| SR068 | ascsucAfaUfUfCfcugaacuaaa | L96 | usUfsuagUfucaggaaUfuGfagusgsa |
| SR819 | (SEQ ID NO. 770) | AWH | (SEQ ID NO. 901) |
| SR070 | gscsccUfuGfGfCfaugaugccua | L96 | usAfsggcAfucaugccAfaGfggcsasc |
| SR800 | (SEQ ID NO. 771) | AWH | (SEQ ID NO. 902) |
| SR074 | csasugCfuCfUfCfgcugggcuga | L96 | usCfsagcCfcagcgagAfgCfaugsasu |
| SR824 | (SEQ ID NO. 772) | AWH | (SEQ ID NO. 903) |
| SR075 | asusagGfgAfUfCfguccucaaaa | L96 | usUfsuugAfggacgauCfcCfuausgsg |
| SR721 | (SEQ ID NO. 773) | AWH | (SEQ ID NO. 904) |
| SR076 | uscsauGfuUfUfGfccaugacaca | L96 | usGfsuguCfauggcaaAfcAfugasusg |
| SR714 | (SEQ ID NO. 774) | AWH | (SEQ ID NO. 905) |
| SR077 | ascsagUfaUfGfGfcaucaugcca | L96 | usGfsgcaUfgaugccaUfaCfugusgsc |
| SR732 | (SEQ ID NO. 775) | AWH | (SEQ ID NO. 906) |
| SR079 | uscsacUfcAfAfUfuccugaacua | L96 | usAfsguuCfaggaauuGfaGfugascsu |
| SR768 | (SEQ ID NO. 776) | AWH | (SEQ ID NO. 907) |
| SR084 | uscscuCfaAfAfUfccaaacggca | L96 | usGfsccgUfuuggauuUfgAfggascsg |
| SR729 | (SEQ ID NO. 777) | AWH | (SEQ ID NO. 908) |
| SR085 | gsgsccAfcAfAfUfacaugcgcua | L96 | usAfsgcgCfauguauuGfuGfgccsgsu |
| SR745 | (SEQ ID NO. 778) | AWH | (SEQ ID NO. 909) |
| SR086 | gscsagUfgUfGfGfccgucacaga | L96 | usCfsuguGfacggccaCfaCfugcsasc |
| SR789 | (SEQ ID NO. 779) | AWH | (SEQ ID NO. 910) |
| SR087 | cscsuuGfcAfCfCfauagggauca | L96 | usGfsaucCfcuaugguGfcAfaggsasa |
| SR724 | (SEQ ID NO. 780) | AWH | (SEQ ID NO. 911) |
| SR088 | ascsaaGfgUfGfCfccuauaaaga | L96 | usCfsuuuAfuagggcaCfcUfuguscsc |
| SR806 | (SEQ ID NO. 781) | AWH | (SEQ ID NO. 912) |
| SR089 | csasuuCfcUfUfGfcaccauagga | L96 | usCfscuaUfggugcaaGfgAfaugsasg |
| SR805 | (SEQ ID NO. 782) | AWH | (SEQ ID NO. 913) |
| SR090 | gsasucAfaGfGfCfucacuuauga | L96 | usCfsauaAfgugagccUfuGfaucsusu |
| SR755 | (SEQ ID NO. 783) | AWH | (SEQ ID NO. 914) |
| SR093 | ascsugCfcUfCfAfcaggaggaua | L96 | usAfsuccUfccugugaGfgCfagusgsg |
| SR760 | (SEQ ID NO. 784) | AWH | (SEQ ID NO. 915) |
| SR099 | ascsccAfaCfUfUfuggcaagcga | L96 | usCfsgcuUfgccaaagUfuGfggusgsg |
| SR727 | (SEQ ID NO. 785) | AWH | (SEQ ID NO. 916) |
| SR100 | gsgscuCfaCfUfUfauggaagcca | L96 | usGfsgcuUfccauaagUfgAfgccsusu |
| SR736 | (SEQ ID NO. 786) | AWH | (SEQ ID NO. 917) |
| SR101 | gsgsccGfuCfAfCfaguucucuca | L96 | usGfsagaGfaacugugAfcGfgccsasc |
| SR814 | (SEQ ID NO. 787) | AWH | (SEQ ID NO. 918) |
| SR102 | asasauAfcCfCfAfaaucccauua | L96 | usAfsaugGfgauuuggGfuAfuuusgsa |
| SR731 | (SEQ ID NO. 788) | AWH | (SEQ ID NO. 919) |
| SR104 | csusgaAfgUfCfAfuuggaccaca | L96 | usGfsuggUfccaaugaCfuUfcagsgsu |
| SR838 | (SEQ ID NO. 789) | AWH | (SEQ ID NO. 920) |
| SR105 | csgsucCfuCfAfAfauccaaacga | L96 | usCfsguuUfggauuugAfgGfacgsasu |
| SR738 | (SEQ ID NO. 790) | AWH | (SEQ ID NO. 921) |
| SR108 | cscsaaCfuUfUfGfgcaagcgcca | L96 | usGfsgcgCfuugccaaAfgUfuggsgsu |
| SR766 | (SEQ ID NO. 791) | AWH | (SEQ ID NO. 922) |
| SR115 | csasugCfgCfUfAfugucaucaaa | L96 | usUfsugaUfgacauagCfgCfaugsusa |
| SR840 | (SEQ ID NO. 792) | AWH | (SEQ ID NO. 923) |
| SR118 | gscsaaGfcGfCfCfccacagacca | L96 | usGfsgucUfguggggcGfcUfugcscsa |
| SR822 | (SEQ ID NO. 793) | AWH | (SEQ ID NO. 924) |
| SR119 | csusguCfcAfAfUfgucuucagua | L96 | usAfscugAfagacauuGfgAfcagsgsu |
| SR801 | (SEQ ID NO. 794) | AWH | (SEQ ID NO. 925) |
| SR123 | usgscaCfaAfGfAfaacggaguca | L96 | usGfsacuCfcguuucuUfgUfgcasgsu |
| SR808 | (SEQ ID NO. 795) | AWH | (SEQ ID NO. 926) |
| SR126 | csasgcAfuUfGfUfgaugaccaca | L96 | usGfsuggUfcaucacaAfuGfcugsasg |
| SR734 | (SEQ ID NO. 796) | AWH | (SEQ ID NO. 927) |
| SR127 | uscsauGfcUfCfUfcgcugggcua | L96 | usAfsgccCfagcgagaGfcAfugasusg |
| SR839 | (SEQ ID NO. 797) | AWH | (SEQ ID NO. 928) |
| SR128 | csgsccCfuGfGfUfggcacaguaa | L96 | usUfsacuGfugccaccAfgGfgcgsasu |
| SR796 | (SEQ ID NO. 798) | AWH | (SEQ ID NO. 929) |
| SR132 | uscscaAfaCfGfGfccacaauaca | L96 | usGfsuauUfguggccgUfuUfggasusu |
| SR764 | (SEQ ID NO. 799) | AWH | (SEQ ID NO. 930) |
| SR135 | csgsgcCfaCfAfAfuacaugcgca | L96 | usGfscgcAfuguauugUfgGfccgsusu |
| SR816 | (SEQ ID NO. 800) | AWH | (SEQ ID NO. 931) |
| SR143 | asusgcGfcUfAfUfgucaucaaga | L96 | usCfsuugAfugacauaGfcGfcausgsu |
| SR825 | (SEQ ID NO. 801) | AWH | (SEQ ID NO. 932) |
| SR146 | usgscgCfuAfUfGfucaucaagga | L96 | usCfscuuGfaugacauAfgCfgcasusg |
| SR810 | (SEQ ID NO. 802) | AWH | (SEQ ID NO. 933) |
| SR147 | asgsugGfuCfUfUfccacugccua | L96 | usAfsggcAfguggaagAfcCfacuscsc |
| SR713 | (SEQ ID NO. 803) | AWH | (SEQ ID NO. 934) |
| SR151 | asgsguGfcCfCfUfauaaaggcaa | L96 | usUfsgccUfuuauaggGfcAfccususg |
| SR804 | (SEQ ID NO. 804) | AWH | (SEQ ID NO. 935) |
| SR153 | uscsccCfuUfGfCfacagccuaga | L96 | usCfsuagGfcugugcaAfgGfggasgsc |
| SR786 | (SEQ ID NO. 805) | AWH | (SEQ ID NO. 936) |
| SR156 | gscsgcUfaUfGfUfcaucaaggga | L96 | usCfsccuUfgaugacaUfaGfcgcsasu |
| SR726 | (SEQ ID NO. 806) | AWH | (SEQ ID NO. 937) |
| SR161 | csasagGfuGfCfCfcuauaaagga | L96 | usCfscuuUfauagggcAfcCfuugsusc |
| SR728 | (SEQ ID NO. 807) | AWH | (SEQ ID NO. 938) |
| SR162 | asusauAfuAfUfGfuuuaacccaa | L96 | usUfsgggUfuaaacauAfuAfuaususg |
| SR843 | (SEQ ID NO. 808) | AWH | (SEQ ID NO. 939) |
| SR169 | gsgsacAfaGfGfUfgcccuauaaa | L96 | usUfsuauAfgggcaccUfuGfuccsusu |
| SR792 | (SEQ ID NO. 809) | AWH | (SEQ ID NO. 940) |
| SR170 | gsuscaUfcAfAfGfggagggauga | L96 | usCfsaucCfcucccuuGfaUfgacsasu |
| SR832 | (SEQ ID NO. 810) | AWH | (SEQ ID NO. 941) |
| SR171 | csasucAfuGfCfUfcucgcuggga | L96 | usCfsccaGfcgagagcAfuGfaugsasa |
| SR833 | (SEQ ID NO. 811) | AWH | (SEQ ID NO. 942) |
| SR172 | gsgsagUfgGfUfCfuuccacugca | L96 | usGfscagUfggaagacCfaCfuccsusu |
| SR775 | (SEQ ID NO. 812) | AWH | (SEQ ID NO. 943) |
| SR174 | asusguCfuUfCfAfgucuggccaa | L96 | usUfsggcCfagacugaAfgAfcaususg |
| SR790 | (SEQ ID NO. 813) | AWH | (SEQ ID NO. 944) |
| SR175 | ascscaUfaGfGfGfaucguccuca | L96 | usGfsaggAfcgaucccUfaUfggusgsc |
| SR777 | (SEQ ID NO. 814) | AWH | (SEQ ID NO. 945) |
| SR177 | usgsgcUfuUfCfUfgcuggguuaa | L96 | usUfsaacCfcagcagaAfaGfccasasu |
| SR780 | (SEQ ID NO. 815) | AWH | (SEQ ID NO. 946) |
| SR181 | csusugUfgCfAfGfuguggccgua | L96 | usAfscggCfcacacugCfaCfaagsasg |
| SR717 | (SEQ ID NO. 816) | AWH | (SEQ ID NO. 947) |
| SR182 | asgsugUfgGfCfCfgucacaguua | L96 | usAfsacuGfugacggcCfaCfacusgsc |
| SR802 | (SEQ ID NO. 817) | AWH | (SEQ ID NO. 948) |
| SR183 | usgscaCfcAfUfAfgggaucguca | L96 | usGfsacgAfucccuauGfgUfgcasasg |
| SR813 | (SEQ ID NO. 818) | AWH | (SEQ ID NO. 949) |
| SR185 | gscsucAfcUfUfAfuggaagccua | L96 | usAfsggcUfuccauaaGfuGfagcscsu |
| SR744 | (SEQ ID NO. 819) | AWH | (SEQ ID NO. 950) |
| SR186 | csasccAfuAfGfGfgaucguccua | L96 | usAfsggaCfgaucccuAfuGfgugscsa |
| SR757 | (SEQ ID NO. 820) | AWH | (SEQ ID NO. 951) |
| SR188 | cscsccUfcCfUfCfuacaugauua | L96 | usAfsaucAfuguagagGfaGfgggsasa |
| SR733 | (SEQ ID NO. 821) | AWH | (SEQ ID NO. 952) |
| SR190 | csusccCfcUfUfGfcacagccuaa | L96 | usUfsaggCfugugcaaGfgGfgagscsa |
| SR829 | (SEQ ID NO. 822) | AWH | (SEQ ID NO. 953) |
| SR191 | csasaaCfgGfCfCfacaauacaua | L96 | usAfsuguAfuuguggcCfgUfuugsgsa |
| SR826 | (SEQ ID NO. 823) | AWH | (SEQ ID NO. 954) |
| SR192 | usgsauGfcCfUfUfuuauuggcua | L96 | usAfsgccAfauaaaagGfcAfucasgsg |
| SR759 | (SEQ ID NO. 824) | AWH | (SEQ ID NO. 955) |
| SR193 | ususgcAfcCfAfUfagggaucgua | L96 | usAfscgaUfcccuaugGfuGfcaasgsg |
| SR739 | (SEQ ID NO. 825) | AWH | (SEQ ID NO. 956) |
| SR198 | asasggAfgUfGfGfucuuccacua | L96 | usAfsgugGfaagaccaCfuCfcuusgsu |
| SR749 | (SEQ ID NO. 826) | AWH | (SEQ ID NO. 957) |
| SR199 | csasugUfuUfGfCfcaugacacca | L96 | usGfsgugUfcauggcaAfaCfaugsasu |
| SR752 | (SEQ ID NO. 827) | AWH | (SEQ ID NO. 958) |
| SR202 | ascsauGfcGfCfUfaugucaucaa | L96 | usUfsgauGfacauagcGfcAfugusasu |
| SR778 | (SEQ ID NO. 828) | AWH | (SEQ ID NO. 959) |
| SR206 | gsgsauGfgAfGfGfcccacaacga | L96 | usCfsguuGfugggccuCfcAfuccsusc |
| SR730 | (SEQ ID NO. 829) | AWH | (SEQ ID NO. 960) |
| SR211 | gscsccUfgGfUfGfgcacaguaua | L96 | usAfsuacUfgugccacCfaGfggcsgsa |
| SR835 | (SEQ ID NO. 830) | AWH | (SEQ ID NO. 961) |
| SR215 | asasggGfaGfGfGfaugaucauca | L96 | usGfsaugAfucaucccUfcCfcuusgsa |
| SR722 | (SEQ ID NO. 831) | AWH | (SEQ ID NO. 962) |
| SR223 | asusuuCfcAfGfUfacaucuauca | L96 | usGfsauaGfauguacuGfgAfaausgsc |
| SR748 | (SEQ ID NO. 832) | AWH | (SEQ ID NO. 963) |
| SR224 | asasuaCfcCfAfAfaucccauuca | L96 | usGfsaauGfggauuugGfgUfauususg |
| SR842 | (SEQ ID NO. 833) | AWH | (SEQ ID NO. 964) |
| SR225 | csasuaGfgGfAfUfcguccucaaa | L96 | usUfsugaGfgacgaucCfcUfaugsgsu |
| SR809 | (SEQ ID NO. 834) | AWH | (SEQ ID NO. 965) |
| SR229 | asuscaAfgGfCfUfcacuuaugga | L96 | usCfscauAfagugagcCfuUfgauscsu |
| SR774 | (SEQ ID NO. 835) | AWH | (SEQ ID NO. 966) |
| SR231 | usgsucCfaAfUfGfucuucaguca | L96 | usGfsacuGfaagacauUfgGfacasgsg |
| SR831 | (SEQ ID NO. 836) | AWH | (SEQ ID NO. 967) |
| SR234 | cscsacAfaUfAfCfaugcgcuaua | L96 | usAfsuagCfgcauguaUfuGfuggscsc |
| SR758 | (SEQ ID NO. 837) | AWH | (SEQ ID NO. 968) |
| SR235 | usasggGfaUfCfGfuccucaaaua | L96 | usAfsuuuGfaggacgaUfcCfcuasusg |
| SR807 | (SEQ ID NO. 838) | AWH | (SEQ ID NO. 969) |
| SR242 | asgsacUfcCfCfAfaggauaaaaa | L96 | usUfsuuuAfuccuuggGfaGfucususg |
| SR823 | (SEQ ID NO. 839) | AWH | (SEQ ID NO. 970) |
| SR246 | csusucAfgUfCfUfggccaugaaa | L96 | usUfsucaUfggccagaCfuGfaagsasc |
| SR803 | (SEQ ID NO. 840) | AWH | (SEQ ID NO. 971) |
| SR249 | asgsgaUfgGfAfGfgcccacaaca | L96 | usGfsuugUfgggccucCfaUfccuscsc |
| SR793 | (SEQ ID NO. 841) | AWH | (SEQ ID NO. 972) |
| SR250 | csascuCfaAfUfUfccugaacuaa | L96 | usUfsaguUfcaggaauUfgAfgugsasc |
| SR818 | (SEQ ID NO. 842) | AWH | (SEQ ID NO. 973) |
| SR251 | gsusucUfcAfUfUfccuugcacca | L96 | usGfsgugCfaaggaauGfaGfaacscsa |
| SR716 | (SEQ ID NO. 843) | AWH | (SEQ ID NO. 974) |
| SR252 | asasggUfgCfCfCfuauaaaggca | L96 | usGfsccuUfuauagggCfaCfcuusgsu |
| SR836 | (SEQ ID NO. 844) | AWH | (SEQ ID NO. 975) |
| SR254 | asascaAfaAfAfUfgaucuacaca | L96 | usGfsuguAfgaucauuUfuUfguususu |
| SR798 | (SEQ ID NO. 845) | AWH | (SEQ ID NO. 976) |
| SR261 | cscsugCfcAfCfCfcaacuuugga | L96 | usCfscaaAfguuggguGfgCfaggsgsu |
| SR715 | (SEQ ID NO. 846) | AWH | (SEQ ID NO. 977) |
| SR267 | usgscaGfuGfUfGfgccgucacaa | L96 | usUfsgugAfcggccacAfcUfgcascsa |
| SR741 | (SEQ ID NO. 847) | AWH | (SEQ ID NO. 978) |
| SR272 | asuscaAfgGfGfAfgggaugauca | L96 | usGfsaucAfucccuccCfuUfgausgsa |
| SR725 | (SEQ ID NO. 848) | AWH | (SEQ ID NO. 979) |
| SR276 | asusccAfaAfCfGfgccacaauaa | L96 | usUfsauuGfuggccguUfuGfgaususu |
| SR737 | (SEQ ID NO. 849) | AWH | (SEQ ID NO. 980) |
| SR279 | csasguGfuGfGfCfcgucacagua | L96 | usAfscugUfgacggccAfcAfcugscsa |
| SR753 | (SEQ ID NO. 850) | AWH | (SEQ ID NO. 981) |
| SR280 | csasagCfgCfCfCfcacagaccua | L96 | usAfsgguCfuguggggCfgCfuugscsc |
| SR794 | (SEQ ID NO. 851) | AWH | (SEQ ID NO. 982) |
| SR281 | csuscaCfuUfAfUfggaagccuaa | L96 | usUfsaggCfuuccauaAfgUfgagscsc |
| SR784 | (SEQ ID NO. 852) | AWH | (SEQ ID NO. 983) |
| SR282 | asasggAfcAfAfGfgugcccuaua | L96 | usAfsuagGfgcaccuuGfuCfcuuscsa |
| SR756 | (SEQ ID NO. 853) | AWH | (SEQ ID NO. 984) |
| SR285 | asgscaUfuUfCfCfaguacaucua | L96 | usAfsgauGfuacuggaAfaUfgcusgsg |
| SR841 | (SEQ ID NO. 854) | AWH | (SEQ ID NO. 985) |
| SR287 | asasuaUfaUfAfUfguuuaaccca | L96 | usGfsgguUfaaacauaUfaUfauusgsa |
| SR773 | (SEQ ID NO. 855) | AWH | (SEQ ID NO. 986) |
| SR289 | gscscaCfcCfAfAfcuuuggcaaa | L96 | usUfsugcCfaaaguugGfgUfggcsasg |
| SR720 | (SEQ ID NO. 856) | AWH | (SEQ ID NO. 987) |
| SR290 | csasugAfuUfUfUfccagcuugga | L96 | usCfscaaGfcuggaaaAfuCfaugsusa |
| SR746 | (SEQ ID NO. 857) | AWH | (SEQ ID NO. 988) |
| SR293 | gsusauGfgCfAfUfcaugccccua | L96 | usAfsgggGfcaugaugCfcAfuacsusg |
| SR791 | (SEQ ID NO. 858) | AWH | (SEQ ID NO. 989) |
| SR298 | ascscuGfaAfGfUfcauuggacca | L96 | usGfsgucCfaaugacuUfcAfggusgsg |
| SR740 | (SEQ ID NO. 859) | AWH | (SEQ ID NO. 990) |
| SR299 | gsascaAfgGfUfGfcccuauaaaa | L96 | usUfsuuaUfagggcacCfuUfgucscsu |
| SR751 | (SEQ ID NO. 860) | AWH | (SEQ ID NO. 991) |
| SR301 | uscsacUfuAfUfGfgaagccuaaa | L96 | usUfsuagGfcuuccauAfaGfugasgsc |
| SR719 | (SEQ ID NO. 861) | AWH | (SEQ ID NO. 992) |
| SR308 | asuscgUfcCfUfCfaaauccaaaa | L96 | usUfsuugGfauuugagGfaCfgauscsc |
| SR811 | (SEQ ID NO. 862) | AWH | (SEQ ID NO. 993) |
| SR313 | csuscaGfcAfUfUfgugaugacca | L96 | usGfsgucAfucacaauGfcUfgagsgsu |
| SR799 | (SEQ ID NO. 863) | AWH | (SEQ ID NO. 994) |
| SR317 | usgsgcCfgGfGfAfacacagacaa | L96 | usUfsgucUfguguuccCfgGfccasasg |
| SR821 | (SEQ ID NO. 864) | AWH | (SEQ ID NO. 995) |
| SR318 | ususgcAfcAfGfCfcuagcccuua | L96 | usAfsaggGfcuaggcuGfuGfcaasgsg |
| SR762 | (SEQ ID NO. 865) | AWH | (SEQ ID NO. 996) |
| SR324 | ascsccUfgCfCfAfcccaacuuua | L96 | usAfsaagUfuggguggCfaGfggusgsa |
| SR776 | (SEQ ID NO. 866) | AWH | (SEQ ID NO. 997) |
| SR329 | csusugCfaCfCfAfuagggaucga | L96 | usCfsgauCfccuauggUfgCfaagsgsa |
| SR820 | (SEQ ID NO. 867) | AWH | (SEQ ID NO. 998) |
| SR331 | gsgsagGfgAfUfGfaucaucauua | L96 | usAfsaugAfugaucauCfcCfuccscsu |
| SR788 | (SEQ ID NO. 868) | AWH | (SEQ ID NO. 999) |
| SR339 | asasuuCfcUfGfAfacuaaucaaa | L96 | usUfsugaUfuaguucaGfgAfauusgsa |
| SR770 | (SEQ ID NO. 869) | AWH | (SEQ ID NO. 1000) |
| SR341 | asgsaaCfuGfCfAfcaagaaacga | L96 | usCfsguuUfcuugugcAfgUfucususg |
| SR785 | (SEQ ID NO. 870) | AWH | (SEQ ID NO. 1001) |
| SR342 | csasccCfaAfCfUfuuggcaagca | L96 | usGfscuuGfccaaaguUfgGfgugsgsc |
| SR754 | (SEQ ID NO. 871) | AWH | (SEQ ID NO. 1002) |
| SR343 | uscsauUfcCfUfUfgcaccauaga | L96 | usCfsuauGfgugcaagGfaAfugasgsa |
| SR781 | (SEQ ID NO. 872) | AWH | (SEQ ID NO. 1003) |
| SR344 | uscsaaAfuCfCfAfaacggccaca | L96 | usGfsuggCfcguuuggAfuUfugasgsg |
| SR830 | (SEQ ID NO. 873) | AWH | (SEQ ID NO. 1004) |

*siRNA modifications:
Nf = 2′-fluoro RNA;
n = 2′-OMe-RNA;
s = phosphorothioate;
(vinu) = vinyl-phosphonate 2′-OMe-uracil

Exemplary siRNAs Targeting Rodent SLC10A1 Transcripts

In some embodiments, the present invention further provides siRNA molecules targeting one or more SLC10A1 mRNA transcripts from one or more species. The siRNA is herein referred to as “surrogate siRNA.” In some embodiments, the surrogate siRNA has cross-reactivities to mouse and rat SLC10A1 mRNA transcripts. In some embodiments, the surrogate siRNA has cross-reactivities to human and rat SLC10A1 mRNA transcripts. In some embodiments, the surrogate siRNA has cross-reactivities to human and mouse SLC10A1 mRNA transcripts. In some embodiments, the surrogate siRNA has cross-reactivities to human, mouse and rat SLC10A1 mRNA transcripts. In some embodiments, the surrogate siRNA has cross-reactivities to human, cynomolgus monkey and mouse SLC10A1 mRNA transcripts. In some embodiments, the surrogate siRNA has cross-reactivities to human, cynomolgus monkey and rat SLC10A1 mRNA transcripts. For example, a surrogate siRNA specifically targets a rodent (e.g., mouse and rat) SLC10A1 mRNA transcript may be used as a research tool to study efficacy and/or target engagement in a rodent model.

In some embodiments, the surrogate siRNA is a 19-mer full match with mouse SLC10A1 mRNA transcript. In some embodiments, the surrogate siRNA is a 19-mer full match with mouse and rat SLC10A1 mRNA transcripts. In some embodiments, the surrogate siRNA is a 19-mer full match with human and mouse SLC10A1 mRNA transcripts. In some embodiments, the surrogate siRNA is a 19-mer full match with human and rat SLC10A1 mRNA transcripts. In some embodiments, the surrogate siRNA is a 19-mer full match with human, cynomolgus monkey and mouse SLC10A1 mRNA transcripts. In some embodiments, the surrogate siRNA is a 19-mer full match with human, cynomolgus monkey and rat SLC10A1 mRNA transcripts.

In some embodiments, a surrogate SLC10A1-targeting siRNA that is a full-match for rodent and human may differ in pharmacological effects for each species, and therefore may be more optimal in rodents than humans.

In some embodiments, the surrogate siRNA is modified with 2′-O-methyl, 2′-F, 5′-vinylphosphonate and phosphorothioate linkages. In some embodiments, the surrogate siRNA is conjugated with the L96 GalNAc on the 3′-end of the sense strand. In some embodiments, the surrogate siRNA is conjugated with the AWH GalNAc on the 3′-end of the sense strand.

Exemplary siRNA molecules targeting rodent SLC10A1 mRNA transcripts comprises a pair of a modified sense strand and a modified antisense strand in Table 6.

TABLE 6
Exemplary modified surrogate siRNAs

| | | GalNAc | | |
|---|---|---|---|---|
| | | at the | | |
| | | 3′end of | Antisense Strand (AS) | |
| Duplex | Sense (S) Strand Sequence | the S | sequence | |
| ID | (5′ to 3′) | strand | (5′ to 3′) | Species |
| SR844 | gsascaAfgGfUfGfcccuacaaaa | L96 | (vinu)sUfsuugUfagggcacCf | Mouse, Rat |
| SR888 | (SEQ ID NO. 1005) | AWH | uUfgucscsu | |
| | | | (SEQ ID NO. 1049) | |
| SR845 | gscsccUfaCfAfAfaggcauuaua | L96 | (vinu)sAfsuaaUfgccuuugUf | Mouse, Rat |
| SR889 | (SEQ ID NO. 1006) | AWH | aGfggcsasc | |
| | | | (SEQ ID NO. 1050) | |
| SR846 | gscscaCfaCfUfAfuguacccuaa | L96 | (vinu)sUfsaggGfuacauagUf | Mouse, Rat |
| SR890 | (SEQ ID NO. 1007) | AWH | gUfggcscsu | |
| | | | (SEQ ID NO. 1051) | |
| SR847 | csasgcAfuCfAfUfguucgucaua | L96 | (vinu)sAfsugaCfgaacaugAf | Mouse, Rat |
| SR891 | (SEQ ID NO. 1008) | AWH | uGfcugsusu | |
| | | | (SEQ ID NO. 1052) | |
| SR848 | gsusucGfuCfAfUfgacaccacaa | L96 | (vinu)sUfsgugGfugucaugAf | Mouse, Rat |
| SR892 | (SEQ ID NO. 1009) | AWH | cGfaacsasu | |
| | | | (SEQ ID NO. 1053) | |
| SR849 | csgsucAfuGfAfCfaccacacuua | L96 | (vinu)sAfsaguGfuggugucAf | Mouse, Rat |
| SR893 | (SEQ ID NO. 1010) | AWH | uGfacgsasa | |
| | | | (SEQ ID NO. 1054) | |
| SR850 | csasgaCfgCfAfCfcaucagcaua | L96 | (vinu)sAfsugcUfgauggugCf | Mouse, Rat |
| SR894 | (SEQ ID NO. 1011) | AWH | gUfcugscsa | |
| | | | (SEQ ID NO. 1055) | |
| SR851 | csusguUfcUfAfCfcauccucaaa | L96 | (vinu)sUfsugaGfgaugguaGf | Mouse, Rat |
| SR895 | (SEQ ID NO. 1012) | AWH | aAfcagsasg | |
| | | | (SEQ ID NO. 1056) | |
| SR852 | gscsguCfaUfUfCfugguaguuaa | L96 | (vinu)sUfsaacUfaccagaaUfg | Mouse |
| SR896 | (SEQ ID NO. 1013) | AWH | Afcgcsusg | |
| | | | (SEQ ID NO. 1057) | |
| SR853 | csgsucAfuUfCfUfgguaguuaua | L96 | (vinu)sAfsuaaCfuaccagaAf | Mouse |
| SR897 | (SEQ ID NO. 1014) | AWH | uGfacgscsu | |
| | | | (SEQ ID NO. 1058) | |
| SR854 | csasuuCfuGfGfUfaguuauguua | L96 | (vinu)sAfsacaUfaacuaccAfg | Mouse |
| SR898 | (SEQ ID NO. 1015) | AWH | Afaugsasc | |
| | | | (SEQ ID NO. 1059) | |
| SR855 | gsgsugAfuCfAfUfcgccauagua | L96 | (vinu)sAfscuaUfggcgaugAf | Mouse |
| SR899 | (SEQ ID NO. 1016) | AWH | uCfaccscsc | |
| | | | (SEQ ID NO. 1060) | |
| SR856 | gsgsccCfaGfUfAfcgguaucaua | L96 | (vinu)sAfsugaUfaccguacUf | Mouse |
| SR900 | (SEQ ID NO. 1017) | AWH | gGfgccsasc | |
| | | | (SEQ ID NO. 1061) | |
| SR857 | csasucUfaCfAfGfcaaaggaaua | L96 | (vinu)sAfsuucCfuuugcugUf | Mouse |
| SR901 | (SEQ ID NO. 1018) | AWH | aGfaugsusa | |
| | | | (SEQ ID NO. 1062) | |
| SR858 | csusacGfaCfGfGfagaucuuaaa | L96 | (vinu)sUfsuaaGfaucuccgUf | Mouse |
| SR902 | (SEQ ID NO. 1019) | AWH | cGfuagsasu | |
| | | | (SEQ ID NO. 1063) | |
| SR859 | cscsuaCfaAfAfGfgcauuaugua | L96 | (vinu)sAfscauAfaugccuuUf | Mouse |
| SR903 | (SEQ ID NO. 1020) | AWH | gUfaggsgsc | |
| | | | (SEQ ID NO. 1064) | |
| SR860 | csusacAfaAfGfGfcauuauguua | L96 | (vinu)sAfsacaUfaaugccuUf | Mouse |
| SR904 | (SEQ ID NO. 1021) | AWH | uGfuagsgsg | |
| | | | (SEQ ID NO. 1065) | |
| SR861 | csascuCfgUfCfAfugguucucaa | L96 | (vinu)sUfsgagAfaccaugaCf | Mouse |
| SR905 | (SEQ ID NO. 1022) | AWH | gAfgugsasu | |
| | | | (SEQ ID NO. 1066) | |
| SR862 | csasggCfaUfGfAfucaucacuua | L96 | (vinu)sAfsaguGfaugaucaUf | Mouse |
| SR906 | (SEQ ID NO. 1023) | AWH | gCfcugscsc | |
| | | | (SEQ ID NO. 1067) | |
| SR863 | gsgscaUfgAfUfCfaucacuuuca | L96 | (vinu)sGfsaaaGfugaugauCf | Mouse |
| SR907 | (SEQ ID NO. 1024) | AWH | aUfgccsusg | |
| | | | (SEQ ID NO. 1068) | |
| SR864 | gscsucUfcUfUfCfcgacuaaaua | L96 | (vinu)sAfsuuuAfgucggaaGf | Mouse |
| SR908 | (SEQ ID NO. 1025) | AWH | aGfagcsasg | |
| | | | (SEQ ID NO. 1069) | |
| SR865 | csgsucCfaAfCfUfcuguucuaca | L96 | (vinu)sGfsuagAfacagaguUf | Mouse |
| SR909 | (SEQ ID NO. 1026) | AWH | gGfacgsusu | |
| | | | (SEQ ID NO. 1070) | |
| SR866 | cscsaaCfuCfUfGfuucuaccaua | L96 | (vinu)sAfsuggUfagaacagAf | Mouse |
| SR910 | (SEQ ID NO. 1027) | AWH | gUfuggsasc | |
| | | | (SEQ ID NO. 1071) | |
| SR867 | cscsggUfgCfUfAfuuugaaaaua | L96 | (vinu)sAfsuuuUfcaaauagCf | Mouse |
| SR911 | (SEQ ID NO. 1028) | AWH | aCfcggsasa | |
| | | | (SEQ ID NO. 1072) | |
| SR868 | gsasacCfuCfAfGfcauugugaua | L96 | (vinu)sAfsucaCfaaugcugAf | Human, Mouse |
| SR912 | (SEQ ID NO. 1029) | AWH | gGfuucsasu | |
| | | | (SEQ ID NO. 1073) | |
| SR869 | csusgaAfgUfCfAfuuggaccaca | L96 | (vinu)sGfsuggUfccaaugaCf | Human, Mouse |
| SR913 | (SEQ ID NO. 1030) | AWH | uUfcagsgsu | |
| | | | (SEQ ID NO. 1074) | |
| SR870 | gsusucUfcAfUfUfccuugcacca | L96 | (vinu)sGfsgugCfaaggaauGf | Human, Rat |
| SR914 | (SEQ ID NO. 1031) | AWH | aGfaacscsa | |
| | | | (SEQ ID NO. 1075) | |
| SR871 | ususcuCfaUfUfCfcuugcaccaa | L96 | (vinu)sUfsgguGfcaaggaaUf | Human, Rat |
| SR915 | (SEQ ID NO. 1032) | AWH | gAfgaascsc | |
| | | | (SEQ ID NO. 1076) | |
| SR872 | csuscaUfuCfCfUfugcaccauaa | L96 | (vinu)sUfsaugGfugcaaggAf | Human, Rat |
| SR916 | (SEQ ID NO. 1033) | AWH | aUfgagsasa | |
| | | | (SEQ ID NO. 1077) | |
| SR873 | ususgcAfcCfAfUfagggaucgua | L96 | (vinu)sAfscgaUfcccuaugGf | Human, Rat |
| SR917 | (SEQ ID NO. 1034) | AWH | uGfcaasgsg | |
| | | | (SEQ ID NO. 1078) | |
| SR874 | usgscaCfcAfUfAfgggaucguca | L96 | (vinu)sGfsacgAfucccuauGf | Human, Rat |
| SR918 | (SEQ ID NO. 1035) | AWH | gUfgcasasg | |
| | | | (SEQ ID NO. 1079) | |
| SR875 | uscscuCfuAfCfAfugauuuucca | L96 | (vinu)sGfsgaaAfaucauguAf | Human, Rat |
| SR919 | (SEQ ID NO. 1036) | AWH | gAfggasgsg | |
| | | | (SEQ ID NO. 1080) | |
| SR876 | uscsuaCfaUfGfAfuuuuccagca | L96 | (vinu)sGfscugGfaaaaucaUf | Human, Rat |
| SR920 | (SEQ ID NO. 1037) | AWH | gUfagasgsg | |
| | | | (SEQ ID NO. 1081) | |
| SR877 | gsgscuGfcAfCfCfauggaguuca | L96 | (vinu)sGfsaacUfccaugguGf | Human, |
| SR921 | (SEQ ID NO. 1038) | AWH | cAfgccscsa | Cynomolgus |
| | | | (SEQ ID NO. 1082) | monkey, Mouse |
| SR878 | csuscaGfcAfUfUfgugaugacca | L96 | (vinu)sGfsgucAfucacaauGf | Human, |
| SR922 | (SEQ ID NO. 1039) | AWH | cUfgagsgsu | Cynomolgus |
| | | | (SEQ ID NO. 1083) | monkey, Mouse |
| SR319 | uscsagCfaUfUfGfugaugaccaa | L96 | (vinu)sUfsgguCfaucacaaUf | Human, |
| SR477 | (SEQ ID NO. 122) | AWH | gCfugasgsg | Cynomolgus |
| | | | (SEQ ID NO. 332) | monkey, Mouse |
| SR879 | csasgcAfuUfGfUfgaugaccaca | L96 | (vinu)sGfsuggUfcaucacaAf | Human, |
| SR923 | (SEQ ID NO. 1040) | AWH | uGfcugsasg | Cynomolgus |
| | | | (SEQ ID NO. 1084) | monkey, Mouse |
| SR880 | uscsauUfcCfUfUfgcaccauaga | L96 | (vinu)sCfsuauGfgugcaagGf | Human, |
| SR924 | (SEQ ID NO. 1041) | AWH | aAfugasgsa | Cynomolgus |
| | | | (SEQ ID NO. 1085) | monkey, |
| | | | | Rat |
| SR881 | csasuuCfcUfUfGfcaccauagga | L96 | (vinu)sCfscuaUfggugcaaGf | Human, |
| SR925 | (SEQ ID NO. 1042) | AWH | gAfaugsasg | Cynomolgus |
| | | | (SEQ ID NO. 1086) | monkey, |
| | | | | Rat |
| SR882 | asusucCfuUfGfCfaccauaggga | L96 | (vinu)sCfsccuAfuggugcaAf | Human, |
| SR926 | (SEQ ID NO. 1043) | AWH | gGfaausgsa | Cynomolgus |
| | | | (SEQ ID NO. 1087) | monkey, |
| | | | | Rat |
| SR883 | cscsuuGfcAfCfCfauagggauca | L96 | (vinu)sGfsaucCfcuaugguGf | Human, |
| SR927 | (SEQ ID NO. 1044) | AWH | cAfaggsasa | Cynomolgus |
| | | | (SEQ ID NO. 1088) | monkey, |
| | | | | Rat |
| SR884 | csusugCfaCfCfAfuagggaucga | L96 | (vinu)sCfsgauCfccuauggUf | Human, |
| SR928 | (SEQ ID NO. 1045) | AWH | gCfaagsgsa | Cynomolgus |
| | | | (SEQ ID NO. 1089) | monkey, |
| | | | | Rat |
| SR885 | gscsacCfaUfAfGfggaucgucca | L96 | (vinu)sGfsgacGfaucccuaUf | Human, |
| SR929 | (SEQ ID NO. 1046) | AWH | gGfugcsasa | Cynomolgus |
| | | | (SEQ ID NO. 1090) | monkey, |
| | | | | Rat |
| SR886 | csasccAfuAfGfGfgaucguccua | L96 | (vinu)sAfsggaCfgaucccuAf | Human, |
| SR930 | (SEQ ID NO. 1047) | AWH | uGfgugscsa | |
| | | | (SEQ ID NO. 1091) | Cynomolgus |
| | | | | monkey, |
| | | | | Rat |
| SR887 | ascscaUfaGfGfGfaucguccuca | L96 | (vinu)sGfsaggAfcgaucccUf | Human, |
| SR931 | (SEQ ID NO. 1048) | AWH | aUfggusgsc | Cynomolgus |
| | | | (SEQ ID NO. 1092) | monkey, |
| | | | | Rat |

*siRNA modifications:
Nf = 2′-fluoro RNA;
n = 2′-OMe-RNA;
s = phosphorothioate;
(vinu) = vinyl-phosphonate 2′-OMe-uracil

### Pharmaceutical Compositions

The siRNA molecules and compositions thereof described herein may be formulated in pharmaceutical compositions. Such a pharmaceutical composition may be provided in a form suitable for administration to a subject, and may be comprise one or more pharmaceutically acceptable carriers, one or more additional ingredients, or some combination of these. The concentrations of the siRNA molecules and pharmaceutical carriers can be determined by experimentation. It should be understood that, although the particles, formulations, compositions and methods in this section are discussed largely with regard to siRNA molecules, these particles, formulations, compositions and methods can be practiced with other modified siRNA compounds.

In some embodiments, the siRNA molecules may be formulated for pharmaceutical use. Pharmaceutically acceptable compositions comprise a therapeutically-effective amount of one or more of the siRNA molecules in any of the preceding embodiments, taken alone or formulated together with one or more pharmaceutically acceptable carriers (additives), excipient and/or diluents.

In some embodiments, the composition is a pharmaceutical composition.

Pharmaceutical compositions that are useful in the methods of the invention may be suitably developed for inhalational, oral, rectal, vaginal, parenteral, topical, transdermal, pulmonary, intranasal, buccal, ophthalmic, intrathecal, transdermal, nasal, inhalation, endotrache, intravenous, subcutaneous, intramuscular, epidural injection, or another route of administration.

In some embodiments, the pharmaceutical compositions may be specially formulated for administration in solid or liquid form, e.g., drenches (aqueous or non-aqueous solutions or suspensions), tablets, sterile solutions, suspensions, sustained-release formulations, cream, ointment, controlled-release patch or spray.

In some embodiments, the pharmaceutical composition is sterile.

Other contemplated formulations include projected nanoparticles, liposomal preparations, rescaled erythrocytes containing the active ingredient, and immunologically-based formulations. The route(s) of administration will be readily apparent to the skilled artisan and will depend upon any number of factors including the type and severity of the disease being treated, the type and age of the veterinary or human patient being treated, and the like.

The formulations of the pharmaceutical compositions described herein may be prepared by any method known or hereafter developed in the art of pharmacology. In general, such preparatory methods include the step of bringing the active ingredient into association with a carrier or one or more other accessory ingredients, and then, if necessary or desirable, shaping or packaging the product into a desired single- or multi-dose unit.

In some embodiments, the compositions of the invention are formulated using one or more pharmaceutically acceptable excipients or carriers. In one embodiment, the pharmaceutical compositions of the invention comprise a therapeutically effective amount of at least one compound of the invention and a pharmaceutically acceptable carrier. Pharmaceutically acceptable carriers, which are useful, include, but are not limited to, glycerol, water, saline, phosphate-buffered saline, ethanol and other pharmaceutically acceptable salt solutions such as phosphates and salts of organic acids. Examples of these and other pharmaceutically acceptable carriers are described in Remington's Pharmaceutical Sciences (1991, Mack Publication Co., New Jersey).

The formulations may be presented in unit dosage form. The amount of active ingredient which can be combined with a carrier material to produce a single dosage form will vary depending upon the host being treated, the particular mode of administration. The amount of active ingredient which can be combined with a carrier material to produce a single dosage form will generally be that amount of the compound which produces a therapeutic effect. The amount of the active ingredient (i.e., a siRNA) may range from about 0.1% to about 99% percent, from about 5% to about 80%, from 10% to 70%, from 20% to 60%, or from 30% to 50%.

In some embodiments, the siRNAs may be formulated in combination with another agent, e.g., another therapeutic agent or an agent that stabilizes the siRNA, e.g., a protein that complexes with the siRNA to form particle.

The compositions disclosed herein can be administered parenterally such as, for example, by intravenous, intramuscular, intrathecal or subcutaneous injection. Parenteral administration can be accomplished by incorporating the compositions into a solution or suspension. Such solutions or suspensions may also include sterile diluents such as water for injection, saline solution, phosphate-buffered saline, fixed oils, polyethylene glycols, glycerine, propylene glycol or other synthetic solvents. Parenteral formulations may also include antibacterial agents such as, for example, benzyl alcohol or methyl parabens, antioxidants such as, for example, ascorbic acid or sodium bisulfite and chelating agents such as EDTA. Buffers such as acetates, citrates or phosphates and agents for the adjustment of tonicity such as sodium chloride or dextrose may also be added. The parenteral preparation can be enclosed in ampules, disposable syringes or multiple dose vials made of glass or plastic.

In one embodiment, the siRNA described herein is formulated with lipid nanoparticle (LNP).

In some embodiments, the compositions are prepared in a solid lyophilized form that can be stored and reconstituted to provide a formulation for administration of treatment. Any process for preparing a solid lyophilized drug product known in the art may be used to prepare the lyophilized products described herein (e.g., U.S. Pat. No. 10,300,018). In some aspects, the lyophilized products are stored until ready to be administration. For example, the lyophilized products comprising a nucleic acid therapeutic described herein can be stored at 4° C. and/or −20° C. In some embodiments, the solid lyophilized products can be reconstituted in sterile water to form a syringible formulation for administration. In some embodiments, the solid lyophilized products can be reconstituted in phosphate buffered saline to form a syringible formulation for administration. Accordingly, the present disclosure further contemplates formulations made by reconstitution of the solid lyophilized products.

### Applications and Methods of Use

In accordance with the present disclosure, the siRNA molecules, and pharmaceutical compositions thereof are used for treating diseases and/or disorders that are associated with NTCP activities, e.g., a cholestatic disorder, hepatitis B, hepatitis D, metabolic dysfunction-associated steatotic liver disease (MASLD), metabolic dysfunction-associated steatohepatitis (MASH), MASLD and increased alcohol intake (MetALD).

In accordance with the present disclosure, the siRNA molecules, and pharmaceutical compositions thereof are used for treating or preventing diseases and/or disorders that may benefit from knockdown of sodium taurocholate co-transporting polypeptide (NTCP), e.g., diabetes, obesity, dyslipidemia, constipation, and inflammatory bowel disease.

In accordance with the present disclosure, the siRNA molecules, and pharmaceutical compositions thereof are used for treating complications of a disease and/or disorder described herein (e.g., in treatment or prevention of a complication or symptom of the disease or disorder). Exemplary complications include any or all of: malnutrition, pruritis, pain, hypertension, progressive liver damage, jaundice, nausea, and/or emesis.

In some embodiments, the siRNA molecules and pharmaceutical compositions thereof are used for treating pain (e.g., pain associated with or resulting from a disease and/or disorder described herein). In embodiments, pain is abdominal pain.

In some embodiments, the siRNA molecules and pharmaceutical compositions thereof are used for treating progressive liver damage (e.g., progressive liver damage associated with or resulting from a disease or disorder described herein). In embodiments, progressive liver damage is liver inflammation, liver fibrosis, liver cirrhosis, and/or liver failure.

In some embodiments, methods for repressing the expression of a SLC10A1 mRNA transcript in a cell, liver or a subject are provided. The method comprises applying to the siRNA of the present disclosure, to the cell, the liver or the subject. The expression repression of the SLC10A1 mRNA transcript in the cell, liver and/or the subject is beneficial for treating a NTCP associated disease (e.g., a cholestatic disorder, hepatitis B, hepatitis D, MAFLD, MASH and MetALD.

The expression of a SLC10A1 mRNA transcript after treatment with a siRNA molecule described herein, may be reduced from about 10% to about 100% as compared to the normal expression level of the hSLC10A1 mRNA transcript before administration. In some embodiments, the SLC10A1 mRNA transcript expression is reduced from about 10% to 95%, or from about 15% to 95%, or from about 20% to 95%, or from about 25% to 95%, or from about 30% to 95%, or from about 35% to 95%, or from about 40% to 95%, or from about 45% to 95%, or from about 50% to 95%, or from 60% to 95%, or from 10% to 85%, or from about 20% to 80%, or from about 25% to 80%, or from about 30% to 80%, or from about 40% to 75%, or from about 45% to 60%. For example, the SLC10A1 mRNA transcript expression in the cell, the liver or the subject is reduced about 10%. In some embodiments, the SLC10A1 mRNA transcript expression in the cell, the liver or the subject is reduced about 15%. In some embodiments, the SLC10A1 mRNA transcript expression in the cell, the liver or the subject is reduced about 20%. In some embodiments, the SLC10A1 mRNA transcript expression in the cell, the liver or the subject is reduced about 25%. In some embodiments, the SLC10A1 mRNA transcript expression in the cell, the liver or the subject is reduced about 30%. In some embodiments, the SLC10A1 mRNA transcript expression in the cell, the liver or the subject is reduced about 35%. In some embodiments, the SLC10A1 mRNA transcript expression in the cell, the liver or the subject is reduced about 40%. In some embodiments, the SLC10A1 mRNA transcript expression in the cell, the liver or the subject is reduced about 45%. In some embodiments, the SLC10A1 mRNA transcript expression in the cell, the liver or the subject is reduced about 50%. In some embodiments, the SLC10A1 mRNA transcript expression in the cell, the liver or the subject is reduced about 55%. In some embodiments, the SLC10A1 mRNA transcript expression in the cell, the liver or the subject is reduced about 60%. In some embodiments, the SLC10A1 mRNA transcript expression in the cell, the liver or the subject is reduced about 65%. In some embodiments, the SLC10A1 mRNA transcript expression in the cell, the liver or the subject is reduced about 70%. In some embodiments, the SLC10A1 mRNA transcript expression in the cell, the liver or the subject is reduced about 75%. In some embodiments, the SLC10A1 mRNA transcript expression in the cell, the liver or the subject is reduced about 80%. In some embodiments, the SLC10A1 mRNA transcript expression in the cell, the liver or the subject is reduced about 85%. In some embodiments, the SLC10A1 mRNA transcript expression in the cell, the liver or the subject is reduced about 90%. In some embodiments, the SLC10A1 mRNA transcript expression in the cell, the liver or the subject is reduced about 95%. In some embodiments, the SLC10A1 mRNA transcript expression in the cell, the liver or the subject is reduced about 96%. In some embodiments, the SLC10A1 mRNA transcript expression in the cell, the liver or the subject is reduced about 97%. In some embodiments, the SLC10A1 mRNA transcript expression in the cell, the liver or the subject is reduced about 98%. In some embodiments, the SLC10A1 mRNA transcript expression in the cell, the liver or the subject is reduced about 99%.

### Cholestatic Disorders

In some embodiments, the present disclosure provides a method of treating a cholestatic disorder in a patient in need; the method comprises administering to the patient the siRNA molecules, pharmaceutical compositions thereof described herein. The cholestatic disorder is selected from the group consisting of progressive intrahepatic familial cholestasis (PFIC), Alagille syndrome (ALGS), biliary atresia (BA), primary biliary cholangitis (PBC), primary sclerosing cholangitis (PSC), and intrahepatic cholestasis of pregnancy (ICP). Other chronic cholestatic disorders include bile acid synthesis defects, cystic fibrosis related liver disease, ductal plate abnormalities including Caroli syndrome and congenital hepatic fibrosis, and certain metabolic diseases.

In some embodiments, provided includes a method for treating PBC and/or PSC in a patient in need thereof comprising administering to the patient in need an effective amount of the siRNA described herein, or a pharmaceutical composition thereof. Primary biliary cholangitis (PBC), previously called primary biliary cirrhosis, and primary sclerosing cholangitis (PSC) are both slow progressive chronic cholestatic liver diseases. PBC is mainly characterized by the granulomatous destruction of small intrahepatic bile ducts, whereas PSC is characterized by inflammation and fibrosis of the intrahepatic and extrahepatic bile ducts, which lead to multiple bile duct stenosis. Both PBC and PSC have autoimmune triggers for bile duct damage, and may lead to cirrhosis and liver failure after a certain period of time.

In some embodiments, provided includes a method for treating PFIC in a patient in need thereof comprising administering to the patient in need an effective amount of the siRNA described herein, or a pharmaceutical composition thereof. PFIC is a pediatric disorder caused by mutations in transporters that control bile flow. PFIC occurs within the first three months of life. There are four types of PFIC: type 1, type 2, type 3, or type 4. Type 1 and Type 2 PFIC are most common. In some embodiments, the methods described herein treat Type 1, Type 2, Type 3, Type 4, Type 5 and/or Type 6 PFIC.

In some embodiments, provided includes a method for treating ALGS in a patient in need thereof comprising administering to the patient in need an effective amount of the siRNA described herein, or a pharmaceutical composition thereof. ALGS is a pediatric disorder caused by mutations in the NOTCH2 and JAG1 genes which result in narrow, malformed, or deficient bile ducts. In some embodiments, a patient with ALGS has a loss of function mutation in JAG1 or NOTCH2. Patients with ALGS exhibit cholestasis and multisystem problems. In some embodiments, patients experience early onset ALGS. Early onset ALGS may occur during infancy.

In some embodiments, provided includes a method for treating biliary atresia (BA) in a patient in need thereof comprising administering to the patient in need an effective amount of the siRNA described herein, or a pharmaceutical composition thereof. BA is a pediatric disorder caused by neuro-inflammatory destructions of intra- or extra-hepatic bile ducts in infants. BA typically occurs between 2 to 8 weeks after birth. Currently, BA is fatal without a Kasai procedure. BA is the number one cause of pediatric liver transplant.

In some embodiments, provided includes a method for treating ICP in a patient in need thereof comprising administering to the patient in need an effective amount of the siRNA described herein, or a pharmaceutical composition thereof. ICP involves a combination of genetic susceptibility, hormonal, and environmental factors. In some embodiments, provided herein is a method of treating a pregnant female patient with ICP.

In some embodiments, provided includes a method for treating cholestasis caused by another disease or procedure in a patient in need thereof comprising administering to the patient in need an effective amount of the siRNA described herein, or a pharmaceutical composition thereof. In some patients receiving a liver transplant, cholestasis after transplantation is observed. In some patients with malignant tumors, biliary obstruction can lead to cholestasis.

In some embodiments, the patient is an adult. In some embodiments, the patient is a pediatric patient.

In some embodiments, provided includes a method for treating disorders and/or diseases in which the knockdown of NTCP may benefit a patient in need thereof, e.g., obesity, diabetes, dyslipidemia, constipation and/or inflammatory bowel disease (IBD), comprising administering to the patient in need an effective amount of the siRNA described herein, or a pharmaceutical composition thereof.

### Hepatitis B and D Infection and Diseases

In some embodiments, the present disclosure provides a method of treating Hepatitis B and/or Hepatitis D in a patient in need; the method comprises administering to the patient the siRNA molecules, pharmaceutical compositions thereof described herein.

In some embodiments, the present disclosure provides a method of curing Hepatitis B and/or Hepatitis D in a patient in need; the method comprises administering to the patient the siRNA molecules, pharmaceutical compositions thereof described herein.

In some embodiments, the patients in need are Hepatitis B antigen (HBeAg)-positive patients. In other embodiments, the patients in need HbeAg-negative patients.

In some embodiments, the treatment continues for at least 3 months, or at least 6 months, at least one year, or life-long. In some embodiments, a long-lasting response after the treatment is discontinued is achieved in the treated patient.

### Other Indications

In some embodiments, the siRNA molecules, and compositions thereof may be used for treating other indications involving bile acid deficiency or associated with NTCP functions. In some embodiments, the present disclosure further provides a method for treating metabolic dysfunction-associated steatotic liver disease (MASLD) in a patient in need comprising administering to the patient in need an effective amount of the siRNA described herein, or a pharmaceutical composition thereof. In some embodiments, the present disclosure further provides a method for treating MASLD and increased alcohol intake (MetALD) in a patient in need comprising administering to the patient in need an effective amount of the siRNA described herein, or a pharmaceutical composition thereof. In other cases, the present disclosure further provides a method for treating metabolic dysfunction-associated steatohepatitis (MASH) in a patient in need comprising administering to the patient in need an effective amount of the siRNA described herein, or a pharmaceutical composition thereof. MAFLD is a condition in which fat builds up in a patient's liver. MASH is an advanced form of MAFLD. MetALD is a form of MASLD associated with increased alcohol intake.

### Combination Therapies

In some embodiments, the present compounds, composition and methods can be used in combination with other active ingredients. The term “in combination” means either a co-therapy or combination therapy, or a co-formulation in a single pharmaceutical form, or in a single commercial package, for example a kit or a blister of two or more active ingredients.

In one aspect, another therapy that targets the bile acid metabolism pathway may be used in combination with the present compound, compositions and/or methods for treating a cholestatic disorder.

In some embodiments, the siRNA molecules, and compositions and methods thereof may be used in combination with one or more therapies for treating PBC in a patient in need, such as Ursodeoxycholic Acid (UDCA), and Obeticholic Acid (OCA).

In some embodiments, the siRNA molecules, and compositions and methods thereof may be used in combination with one or more therapies for treating PSC in a patient in need, such as UDCA.

In some embodiments, the siRNA molecules, and compositions and methods thereof may be used in combination with one or more therapies for treating PFIC in a patient in need, such as UDCA, IBAT (ileal bile acid transport) inhibitors and/or in combination with a surgical treatment of PFIC (e.g., partial external biliary diversion (PEBD)).

In some embodiments, the siRNA molecules, and compositions and methods thereof may be used in combination with one or more therapies for treating Alagille syndrome (ALGS), such as UDCA that improves the flow of bile from the liver to the small intestine, and/or other medicines that can help to treat liver and itch skin symptoms in patients with ALGS such as IBAT inhibitors.

In some embodiments, the siRNA molecules, and compositions and methods thereof may be used in combination with other treatment methods of biliary atresia (BA) in a patient in need, such as a surgical procedure called Kasai procedure.

In some embodiments, the siRNA molecules, and compositions and methods thereof may be used in combination with one or more medications for treating intrahepatic cholestasis of pregnancy (ICP) in a patient in need, such as UDCA.

Other therapies for treating a cholestatic disorder include Nor-UDCA, cholic acid, Rifampin, Bile acid sequestrants, chemical chaperones, IBAT (ileal bile acid transport) inhibitors, FXR and TGR5 agonists, FGF19 analog, and anti-inflammatory therapies.

The IBAT inhibitors include eloixibat, linerixibat, odevixibat, maralixibat and the like.

In another aspect, the siRNA molecules, compositions and methods as described herein may be used in combination with one or more anti-viral therapies for treating or curing hepatitis B and/or hepatitis D.

In some embodiments, the siRNA molecules, compositions and methods as described herein may be used in combination with one or more antiviral agents, including but not limited to interferons (for example Intron A, Pegasys), nucleoside and nucleotide analogs that interfere with DNA polymerases (for example Lamivudine, Adefovir, tenofovir, entecavir and the like), siRNA drugs that target part of the viral genome (for example VIR-2218, RG6346, JNJ-3989 and the like), entry inhibitors that may be additive or potentially synergistic with the siRNA described herein (for example beluvirtide, hzVSF, Albireo 2342, AB-543 and the like), capsid or core inhibitors (for example morphothiadin, JNJ 56136379, EDP-514 and the like), S-antigen inhibitors (for example REP 2139, BJT-574 and the like), antisense molecules that target the viral mRNA (such as bepirovirsen and the like), gene editing agents, therapeutic vaccines, monoclonal antibodies that neutralize HBV or HDV proteins, and prenylation inhibitors (such as lonafarnib).

In some embodiments, a subject in need may receive a preventive vaccine against hepatitis B and/or D. The subject may further receive a treatment with the siRNA molecules and pharmaceutical compositions thereof as described herein. The preventive vaccine may include but are not limited to hepatitis B vaccine, hepatitis D vaccine and hepatitis B immunoglobulin.

In some embodiments, the siRNA molecules, compositions and methods as described herein may be used in combination with one or more viral suppressive medications. Exemplary antiviral medications for hepatitis B include entecavir, tenofovir, lamivudine, adefovir and telbivudine. In some cases, the antiviral medications are nucleos(t)ide analogs (Nas). In some cases, the antiviral medications are peginterferon (PEG-IFN).

In some embodiments, the siRNA molecules, compositions and methods as described herein may be used in combination with one or more immunomodulatory therapies. As immune response is suppressed in chronic HBV infection, restoration of the immune response is crucial for improving the chronic infection outcome of hepatitis viral infection, which can be achieved using immunomodulators.

Exemplary immunomodulatory therapies include pegylated Interferon alfa-2b (Intron A), PD-1, PD-L1 or other immune checkpoint inhibitors, Toll-like receptor (TLR) agonists, recombinant interleukins, antibodies and agonists of pattern recognition receptors or other general or specific immunomodulatory agents.

In some embodiments, the immunomodulators are TLR agonists. TLR agonists have attracted interest for use as vaccine adjuvants or immune modulators because of their ability to induce the production of IFN, proinflammatory cytokines and chemokines, which exert anti-HBV effect. Exemplary TLR agonists include TLR 2 agonists, TLR4 agonists, TLR7 agonists (e.g., GS-9620 (vesatolimod), APR002 and AL-034), TLR8 agonists (e.g., ssRNA40 and GS-9688), and TLR9 agonists (e.g., CpG oligodeoxynucleotides (ODNs) and AIC649). Other agents that can stimulate the innate immune response may include STING agonists and RIG-I (retinoic acid-inducible protein) agonists.

In other embodiments, the siRNA described herein or the composition thereof is used optionally with additional antiviral agents in combination with immunomodulators that may activate the immune response in the host in a manner to improve the response to the viral infection. The immunomodulatory agents include but are not limited to therapeutic vaccines to stimulate the immune system (for example HerberNasvac, HepTCell, VBI-2601, GSK3528869A and the like), TLR agonists (such as TLR4, 7, 8 and 9 including but not limited to Selgantolimod/GS9688, RG7854, CB06, imiquimod, SBT8230 and the like), Monoclonal antibodies that neutralize HBV proteins and may or may not have FC domain signaling capabilities (such as lenvervimab, VIR-3434, BJT-778 and the like), check point inhibitors (for example (ASC22, GS4224, RG6084, Keytruda, anti-CTLA-4, anti-LAG3, anti-PD-1, anti-PDL1, anti-TIGIT, anti-ICOS and the like) as well as additional immune system adjuvants and modulatory agents including potentially cell therapies. While the above list is not meant to be limiting or exhaustive, one skilled in the art will realize there are extensive immunomodulatory mechanisms that could benefit from the siRNA therapy described herein to reduce viral load and replication. Combinations of multiple immunomodulatory agents may be employed potentially with combinations of additional antiviral therapies to result in a functional and/or sterilizing cure of viral hepatitis B and D.

In some embodiments, the siRNA molecules, compositions and methods as described herein may be used in combination with one or more therapeutic HBV or HDV vaccines. The individual patient's own immune response to HBV infection is important for spontaneous clearance of HBV infection requires a vigorous, polyclonal antigen-specific, adaptive immune response against HBV proteins. In some instances, after treatment with the siRNA molecule of the present disclosure, or a composition thereof after an acute viral infection, a therapeutic vaccine may be used to clear the viral proteins.

In yet another aspect, the present compound, compositions and methods as described herein may be used in combination with another therapy of MAFLD, MASH, or MetALD.

In some embodiments, two or more compositions are administered simultaneously, sequentially, or at an interval period of time. In some embodiments, one or more pharmaceutical compositions are administered simultaneously. In some cases, one or more pharmaceutical compositions are administered sequentially. In additional cases, one or more pharmaceutical compositions are administered at an interval period of time (e.g., the first administration of a first pharmaceutical composition is on day one followed by an interval of at least 1, 2, 3, 4, 5, or more days prior to the administration of at least a second pharmaceutical composition.

### Kits and Packages

In another aspect, the present disclosure provides kits and commercial packages comprising one or more siRNA molecules for repressing the expression of a SLC10A1 mRNA transcript, and optionally one or more agents that supplement use of the siRNA molecules in the kits and packages, such as buffers.

In some embodiments, the kit may also contain positive and negative control siRNAs (e.g., a non-targeting control siRNA or a siRNA that targets an unrelated mRNA). The kit also may contain reagents for assessing knockdown of the intended target gene SLC10A1 (e.g., primers and probes for quantitative PCR to detect the target mRNA and/or antibodies against the corresponding protein for western blots).

In some embodiments, the kit may comprise a siRNA sequence information and the instructions and materials necessary to generate the siRNA.

In some embodiments, a pharmaceutical combination in kit form may be provided that includes, in packaged combination, a carrier means adapted to receive a container means in close confinement therewith and a first container means including an interfering RNA composition and an acceptable carrier. Such kits can further include, if desired, one or more of various conventional pharmaceutical kit components, such as, for example, containers with one or more pharmaceutically acceptable carriers, additional containers, etc., as will be readily apparent to those skilled in the art. Printed instructions, either as inserts or as labels, indicating quantities of the components to be administered, guidelines for administration, and/or guidelines for mixing the components, can also be included in the kit.

### EXAMPLES

### Example 1: Identification of siRNAs Targeting Human SLC10A1 mRNA

Candidate sense and antisense strands of siRNAs including all possible siRNAs were designed for human SLC10A1 mRNA transcript (NM_003049.4; SEQ ID NO. 1093). The sequences were calculated and selected using selection criteria including frequency of off-targets, homology with miRNA seed sequences and cross reactivity with SLC10A1 mRNAs from other species, etc. The sense strand and antisense strand sequences were calculated separately for the selection criteria. siRNA off-target genes were predicted for human, rhesus monkey, cynomolgus monkey, mouse and rat SLC10A1 mRNA transcripts. siRNA strands were analyzed for presence of any of miRNA seed regions in human, rhesus monkey, dog, pig, mouse, rat and rabbit miRNAs. The cross-reactivity of each siRNA candidate was calculated for transcript variants and difference species. The top candidate sequences were selected and mapped to human SLC10A1 mRNA transcript after the calculations. Upon completion of the bioinformatic selection described herein, preferred siRNA-GalNAc conjugated candidate sequences were synthesized for further evaluation.

### Example 2: Silencing SLC10A1 Expression Using siRNAs

Each GalNAc conjugated siRNA molecule was evaluated in primary human hepatocytes at 0.3 μM or 1 μM with 3 biological replicates. To do this, primary human hepatocytes were isolated from a normal human liver, cryopreserved, and stored in liquid nitrogen until ready for experimental plating. Primary hepatocytes were thawed and plated in a collagen gel layer at 220,000 cells/cm² and treated with GalNAc-conjugated siRNAs prior to attachment hepatocyte attachment. The presence of the GalNAc moiety facilitates uptake in primary human hepatocytes without transfection reagents. After plating, cells were maintained in a humidified incubator at 37° C. and 5% CO₂ for the remainder of the experiment. The cultures were left overnight in maintenance medium that consisted of DMEM/F-12 supplemented with 10% fetal bovine serum, 50 mg/ml gentamycin, 0.2% ITS (Fisher/MediaTech MT-25c800CR), and dexamethasone (Sigma Aldrich D4902; 1 mM at plating and 250 nM thereafter). The following day, the media was exchanged for fresh maintenance media without GalNAc siRNAs. 72 hours after initial treatment with the GalNAc siRNAs, hepatocytes' RNA was collected in trizol using manufacturer protocols.

RNA was isolated using the Invitrogen Purelink RNA Mini kit (Cat. No. 12183018A) according to manufacturer's instructions. Measurement of SLC10A1 mRNA levels utilized a real-time reverse transcriptase polymerase chain reaction (RT-PCR) in duplicate. RNA was multiplexed with probes specific for human SLC10A1 and a human RPS11 using a BioRad CFX96 PCR Machine. For determining the percent gene knockdown by the GalNAc siRNAs, cycle times for SLC10A1 were normalized per well to RPS11 (a control, house-keeping gene) and then presented as percent inhibition relative to the negative, non-targeting control. Table 7 shows the percent knockdown of 146 selected siRNAs in primary hepatocytes.

TABLE 7
knockdown of expression of SLC10A1 mRNA by siRNA duplexes

| | Percent | | Percent | |
|---|---|---|---|---|
| | knockdown | Adj. | knockdown | Adj. |
| Duplex | (0.3 μM) | p-value | (1.0 μM) | p-value |
| ID | (%) | (0.3 μM) | (%) | (1 μM) |
| SR002 | −4% | 1.000 | −14% | 1.000 |
| SR003 | −5% | 1.000 | 14% | 1.000 |
| SR005 | 4% | 1.000 | −7% | 1.000 |
| SR013 | 17% | 1.000 | 26% | 1.000 |
| SR016 | 3% | 1.000 | −10% | 1.000 |
| SR018 | −3% | 1.000 | 9% | 1.000 |
| SR019 | 55% | <0.001 | 46% | <0.001 |
| SR024 | 31% | 0.002 | 30% | 1.000 |
| SR025 | −13% | 1.000 | −25% | 1.000 |
| SR026 | −1% | 1.000 | −9% | 1.000 |
| SR028 | 67% | <0.001 | 73% | <0.001 |
| SR030 | 40% | <0.001 | 44% | 0.084 |
| SR033 | −16% | 1.000 | −13% | 1.000 |
| SR034 | 3% | 1.000 | −29% | 1.000 |
| SR035 | 0% | 1.000 | 23% | 1.000 |
| SR036 | −16% | 1.000 | −37% | 0.901 |
| SR037 | 26% | 0.037 | 31% | 1.000 |
| SR043 | 22% | 0.315 | 18% | 1.000 |
| SR045 | 14% | 1.000 | 13% | 1.000 |
| SR046 | 7% | 1.000 | −13% | 1.000 |
| SR047 | 61% | <0.001 | 73% | <0.001 |
| SR048 | −2% | 1.000 | −9% | 1.000 |
| SR049 | −16% | 1.000 | −35% | 1.000 |
| SR050 | 28% | 0.010 | 32% | 0.141 |
| SR051 | −14% | 1.000 | −15% | 1.000 |
| SR057 | −7% | 1.000 | −13% | 1.000 |
| SR059 | 51% | <0.001 | 51% | 0.004 |
| SR062 | 27% | 0.019 | 34% | 0.057 |
| SR064 | −9% | 1.000 | 2% | 1.000 |
| SR067 | 4% | 1.000 | −12% | 1.000 |
| SR068 | 47% | <0.001 | 44% | 0.089 |
| SR070 | 0% | 1.000 | −11% | 1.000 |
| SR074 | −24% | 0.970 | −18% | 1.000 |
| SR075 | 23% | 0.253 | 9% | 1.000 |
| SR076 | 47% | <0.001 | 43% | <0.001 |
| SR077 | 15% | 1.000 | −2% | 1.000 |
| SR079 | 31% | 0.002 | 28% | 1.000 |
| SR082 | 52% | <0.001 | 61% | <0.001 |
| SR084 | 5% | 1.000 | 16% | 1.000 |
| SR085 | −20% | 1.000 | −16% | 1.000 |
| SR086 | −6% | 1.000 | 8% | 1.000 |
| SR087 | −13% | 1.000 | −15% | 1.000 |
| SR088 | −32% | 0.118 | −21% | 1.000 |
| SR089 | −2% | 1.000 | −16% | 1.000 |
| SR090 | 63% | <0.001 | 57% | <0.001 |
| SR093 | −1% | 1.000 | 20% | 1.000 |
| SR099 | 5% | 1.000 | −19% | 1.000 |
| SR100 | 6% | 1.000 | −24% | 1.000 |
| SR101 | −10% | 1.000 | 12% | 1.000 |
| SR102 | 15% | 1.000 | 26% | 1.000 |
| SR104 | 47% | <0.001 | 48% | 0.016 |
| SR105 | 9% | 1.000 | −3% | 1.000 |
| SR108 | 18% | 1.000 | −2% | 1.000 |
| SR115 | 26% | 0.047 | 38% | 0.013 |
| SR118 | −19% | 1.000 | 3% | 1.000 |
| SR119 | −1% | 1.000 | −3% | 1.000 |
| SR123 | 6% | 1.000 | 15% | 1.000 |
| SR126 | 14% | 1.000 | 8% | 1.000 |
| SR127 | −6% | 1.000 | 9% | 1.000 |
| SR128 | 12% | 1.000 | 5% | 1.000 |
| SR132 | −22% | 1.000 | −11% | 1.000 |
| SR135 | 12% | 1.000 | 6% | 1.000 |
| SR143 | −1% | 1.000 | −4% | 1.000 |
| SR146 | 14% | 1.000 | 68% | <0.001 |
| SR147 | 5% | 1.000 | 32% | 0.178 |
| SR151 | −44% | 0.002 | −27% | 1.000 |
| SR153 | −27% | 0.450 | −32% | 1.000 |
| SR156 | −10% | 1.000 | −5% | 1.000 |
| SR158 | 69% | <0.001 | 74% | <0.001 |
| SR159 | 58% | <0.001 | 48% | 0.017 |
| SR161 | −28% | 0.356 | −47% | 0.169 |
| SR162 | 24% | 0.144 | 23% | 1.000 |
| SR167 | 84% | <0.001 | 82% | <0.001 |
| SR169 | −34% | 0.062 | −8% | 1.000 |
| SR170 | −38% | 0.018 | −27% | 1.000 |
| SR171 | −32% | 0.109 | −52% | 0.069 |
| SR172 | −12% | 1.000 | −13% | 1.000 |
| SR174 | 31% | 0.002 | 55% | <0.001 |
| SR175 | 0% | 1.000 | −3% | 1.000 |
| SR177 | 25% | 0.090 | 26% | 1.000 |
| SR181 | 16% | 1.000 | 16% | 1.000 |
| SR182 | −13% | 1.000 | −8% | 1.000 |
| SR183 | −1% | 1.000 | −8% | 1.000 |
| SR185 | 14% | 1.000 | 7% | 1.000 |
| SR186 | −21% | 1.000 | −3% | 1.000 |
| SR188 | 24% | 0.103 | 33% | 1.000 |
| SR190 | −37% | 0.028 | −39% | 0.621 |
| SR191 | 8% | 1.000 | 13% | 1.000 |
| SR192 | 24% | 0.482 | 33% | 0.362 |
| SR193 | −28% | 0.338 | −40% | 1.000 |
| SR195 | 58% | <0.001 | 67% | <0.001 |
| SR197 | 49% | <0.001 | 59% | <0.001 |
| SR198 | −15% | 1.000 | −32% | 1.000 |
| SR199 | 18% | 1.000 | 31% | 0.262 |
| SR202 | −15% | 1.000 | −33% | 1.000 |
| SR206 | −1% | 1.000 | 5% | 1.000 |
| SR211 | −27% | 0.477 | −9% | 1.000 |
| SR215 | 47% | <0.001 | 47% | <0.001 |
| SR223 | 35% | <0.001 | 32% | 1.000 |
| SR224 | −19% | 1.000 | −4% | 1.000 |
| SR225 | 1% | 1.000 | −5% | 1.000 |
| SR229 | −14% | 1.000 | −25% | 1.000 |
| SR231 | 24% | 0.109 | 58% | <0.001 |
| SR234 | 39% | <0.001 | 30% | 0.901 |
| SR235 | 47% | <0.001 | 40% | 0.299 |
| SR237 | 54% | <0.001 | 56% | <0.001 |
| SR240 | 54% | <0.001 | 70% | <0.001 |
| SR242 | 18% | 1.000 | 17% | 1.000 |
| SR246 | −27% | 0.477 | −12% | 1.000 |
| SR249 | −34% | 0.061 | −4% | 1.000 |
| SR250 | 25% | 0.059 | 38% | 0.470 |
| SR251 | −35% | 0.049 | −49% | 0.152 |
| SR252 | −28% | 0.299 | −22% | 1.000 |
| SR254 | −32% | 0.118 | −14% | 1.000 |
| SR259 | 54% | <0.001 | 56% | <0.001 |
| SR261 | 1% | 1.000 | −16% | 1.000 |
| SR267 | 10% | 1.000 | 11% | 1.000 |
| SR271 | 85% | <0.001 | 90% | <0.001 |
| SR272 | 6% | 1.000 | 7% | 1.000 |
| SR276 | −2% | 1.000 | 7% | 1.000 |
| SR279 | 24% | 0.118 | 14% | 1.000 |
| SR280 | 19% | 1.000 | 32% | 0.166 |
| SR281 | 5% | 1.000 | 12% | 1.000 |
| SR282 | −28% | 0.356 | −38% | 0.787 |
| SR285 | 57% | <0.001 | 48% | 0.013 |
| SR287 | −10% | 1.000 | −34% | 1.000 |
| SR289 | 13% | 1.000 | 14% | 1.000 |
| SR290 | −16% | 1.000 | 8% | 1.000 |
| SR293 | −7% | 1.000 | −7% | 1.000 |
| SR297 | 42% | <0.001 | 49% | 0.012 |
| SR298 | 3% | 1.000 | 15% | 1.000 |
| SR299 | −43% | 0.003 | −31% | 1.000 |
| SR301 | −6% | 1.000 | −9% | 1.000 |
| SR308 | −39% | 0.011 | −23% | 1.000 |
| SR313 | −32% | 0.118 | −26% | 1.000 |
| SR317 | 16% | 1.000 | 3% | 1.000 |
| SR318 | −5% | 1.000 | −16% | 1.000 |
| SR324 | −15% | 1.000 | −10% | 1.000 |
| SR329 | 46% | <0.001 | 55% | <0.001 |
| SR331 | 30% | 0.037 | 40% | 0.028 |
| SR332 | 61% | <0.001 | 74% | <0.001 |
| SR339 | 32% | <0.001 | 45% | 0.061 |
| SR341 | 21% | 0.482 | 15% | 1.000 |
| SR342 | 21% | 0.480 | 16% | 1.000 |
| SR343 | −22% | 1.000 | −27% | 1.000 |
| SR344 | 8% | 1.000 | 20% | 1.000 |

The GalNAc siRNAs that were able to provide the best percent knockdown of SLC10A1 were further evaluated in dose response curves 1 μM to 16.9 pM following the same procedure above. Table 8 shows the dose response curves of the 15 siRNAs out of the 146 candidates.

TABLE 8
IC₅₀ of siRNA duplexes

| | Max % Inhibition | | IC₅₀ |
|---|---|---|---|
| Duplex ID | Average | SD | (nM) |
| SR019 | 64.1 | 7.6 | 12.1 |
| SR028 | 74.3 | 5.0 | 21.6 |
| SR047 | 74.2 | 4.5 | 28.0 |
| SR082 | 73.4 | 2.1 | 26.0 |
| SR158 | 87.2 | 3.7 | 9.0 |
| SR159 | 61.5 | 1.2 | 22.3 |
| SR167 | 89.4 | 3.6 | 3.7 |
| SR195 | 77.7 | 1.0 | 8.2 |
| SR197 | 70.9 | 8.9 | 12.1 |
| SR237 | 79.4 | 4.7 | 13.0 |
| SR240 | 63.5 | 2.9 | 39.3 |
| SR259 | 78 | 3.7 | 16.6 |
| SR271 | 81.5 | 4.0 | 2.4 |
| SR297 | 59.8 | 4.3 | 4.8 |
| SR332 | 75.1 | 1.8 | 16.1 |

### Example 3: Modification of siRNAs

For the GalNAc siRNAs described in Table 8 that provided the best percent knockdowns and most potent IC₅₀s, an additional 13 siRNAs with varied backbone modifications were synthesized for each sequence based on known literature modifications found to improve siRNA stability, off-target safety, and uptake. The modified sense and antisense strands of siRNA molecules are shown in Tables 3 and 5. These additional GalNAc siRNAs were evaluated in vitro in uptake assays in primary human hepatocytes (Donor L) to determine the percent knockdown of SLC10A1 at two concentrations (5 and 50 nM, Table 9) or IC₅₀ values (Table 10), as described above.

TABLE 9
knockdown of SLC10A1 mRNA expression by backbone modified
(SAR) siRNA duplexes at two concentrations

| | Percent (%) Knockdown of | | | Percent (%) Knockdown of | |
|---|---|---|---|---|---|
| | SLC10A1 mRNA per siRNA | | | SLC10A1 mRNA per siRNA | |
| | concentration | | | concentration | |
| Duplex ID | 5 nM | Duplex ID | Duplex ID | 5 nM | 50 nM |
| SR001 | 8.45 | 63.9 | SR195 | 32.7 | 68.7 |
| SR004 | 52.2 | 70.8 | SR196 | 49.9 | 67.5 |
| SR007 | −94.7 | 49.5 | SR197 | 38.8 | 63.5 |
| SR008 | 47.1 | 68 | SR200 | 37.9 | 60.9 |
| SR011 | 28.5 | 51.3 | SR201 | 29.1 | 21.7 |
| SR012 | 20.8 | 62.6 | SR203 | 14 | 57.4 |
| SR014 | 53.3 | 69.7 | SR205 | 41.5 | 67.3 |
| SR017 | 24.9 | 50.6 | SR207 | 46.7 | 73.5 |
| SR019 | 43.1 | 65.9 | SR208 | 32.8 | 60.4 |
| SR020 | 32.9 | 49 | SR209 | 36.7 | 60.9 |
| SR021 | 23 | 52.5 | SR210 | 28.7 | 60.4 |
| SR022 | 44.9 | 75 | SR213 | 53.5 | 68.3 |
| SR027 | 24.6 | 45.3 | SR214 | −19.8 | 50 |
| SR028 | 5.47 | 24.7 | SR216 | 33.6 | 57.4 |
| SR029 | 22.9 | 47.1 | SR217 | 39.2 | 59.1 |
| SR031 | 46.7 | 65.8 | SR218 | −86.4 | 46.8 |
| SR032 | 47.8 | 61.2 | SR219 | −50.1 | 58.2 |
| SR038 | 38.5 | 72 | SR220 | 40 | 49.9 |
| SR039 | 39.4 | 56.7 | SR221 | 45 | 65.4 |
| SR040 | −9.48 | 23.7 | SR226 | 46.1 | 57.7 |
| SR041 | 57 | 77.6 | SR227 | 26.3 | 56.4 |
| SR042 | 17.6 | 57.1 | SR228 | 46.6 | 69.9 |
| SR044 | 48.6 | 74 | SR230 | 19.2 | 44.2 |
| SR047 | 3.83 | 37.2 | SR233 | 36.6 | 63.9 |
| SR053 | 29.3 | 48.2 | SR237 | 41.9 | 76.2 |
| SR054 | 3.12 | 48.1 | SR238 | 43.4 | 70.1 |
| SR058 | 13.2 | 57 | SR239 | 13.7 | 52.5 |
| SR061 | 34.7 | 57.8 | SR240 | −5.83 | 2.29 |
| SR063 | 48.1 | 58.9 | SR243 | 35 | 62.1 |
| SR065 | 28.7 | 67.3 | SR245 | 47.1 | 64.8 |
| SR066 | 37.6 | 64.1 | SR247 | 40.4 | 61.3 |
| SR071 | 58 | 77.6 | SR248 | 40.7 | 66.6 |
| SR073 | 44 | 69.1 | SR253 | 48.4 | 71.2 |
| SR078 | 38.9 | 53.7 | SR255 | 59.7 | 63.3 |
| SR080 | 50.7 | 69.9 | SR256 | 40.2 | 68.6 |
| SR081 | 42.4 | 67.3 | SR257 | 30.6 | 67.3 |
| SR082 | 10.5 | 54.5 | SR258 | −27 | 23.9 |
| SR083 | 46.5 | 63.6 | SR259 | 37.1 | 59.7 |
| SR091 | 38.9 | 71.1 | SR260 | 29.7 | 61 |
| SR092 | 33.7 | 55.9 | SR262 | −157 | 16.8 |
| SR094 | 27.9 | 61.3 | SR263 | 7.78 | 33.9 |
| SR095 | 35.6 | 61.2 | SR265 | 52.9 | 81.2 |
| SR096 | 35.1 | 55.4 | SR266 | 37.4 | 62.5 |
| SR097 | 32 | 52.1 | SR269 | 15.4 | 57.5 |
| SR098 | 55.8 | 72.2 | SR270 | 51.9 | 69.9 |
| SR106 | 25.8 | 54.1 | SR273 | 39.6 | 59.1 |
| SR107 | 13.5 | 36.3 | SR277 | −1.8 | 54.9 |
| SR109 | 22.2 | 51.4 | SR278 | 51.3 | 67.9 |
| SR110 | 42 | 70.7 | SR283 | 55.3 | 70.3 |
| SR112 | −43.2 | 50.5 | SR284 | 39.9 | 64.2 |
| SR113 | −4.97 | 66.5 | SR286 | 30.8 | 62.4 |
| SR114 | 39.2 | 28.1 | SR291 | 45.1 | 66.1 |
| SR116 | 36.2 | 67.6 | SR292 | 19.4 | 63.2 |
| SR117 | 53.3 | 73.6 | SR294 | 45.1 | 71.4 |
| SR120 | 38.7 | 57.9 | SR295 | 11.2 | 60.5 |
| SR121 | 31.6 | 52.2 | SR296 | 23.9 | 58.2 |
| SR124 | 36.6 | 57.2 | SR297 | 1.65 | 40.5 |
| SR125 | 35.6 | 58.4 | SR300 | 26.8 | 64.4 |
| SR129 | 53.8 | 72.7 | SR304 | 23.9 | 65.2 |
| SR133 | 19.5 | 67.3 | SR305 | 45 | 66.3 |
| SR136 | 35.9 | 57 | SR306 | −64 | 68.2 |
| SR137 | 31 | 45.1 | SR307 | 43.7 | 67.2 |
| SR138 | 24.2 | 63.2 | SR309 | 24.7 | 49.9 |
| SR139 | 32 | 69.7 | SR310 | 48.1 | 59.7 |
| SR141 | −7.6 | 59.3 | SR311 | 35.9 | 58.1 |
| SR144 | 35.2 | 63.8 | SR312 | 33.2 | 54.6 |
| SR148 | 4.76 | 50.9 | SR314 | 36.1 | 54 |
| SR150 | 29.2 | 72.6 | SR315 | 17.5 | −1.91 |
| SR152 | 41 | 56.3 | SR316 | 2.29 | 54.3 |
| SR154 | 40.9 | 54.9 | SR319 | 59.7 | 74.3 |
| SR157 | 31.1 | 57.7 | SR321 | 29.2 | 63.9 |
| SR159 | −8.07 | 44.7 | SR322 | 17.9 | 32 |
| SR160 | 53.2 | 71 | SR323 | 30.4 | 71 |
| SR163 | 48.8 | 67.2 | SR325 | 49.4 | 69 |
| SR164 | 46.3 | 62.7 | SR326 | 41.6 | 65.9 |
| SR165 | 42.9 | 65.8 | SR327 | 36.8 | 60.4 |
| SR166 | 40.5 | 58.1 | SR330 | 20.1 | 50.8 |
| SR168 | 44 | 66.7 | SR332 | 6.38 | 41.9 |
| SR173 | 37.3 | 58.1 | SR333 | 50.6 | 74.2 |
| SR176 | 32.1 | 58 | SR335 | 13.7 | 45.5 |
| SR179 | 40.5 | 67.7 | SR337 | 55.3 | 70.2 |
| SR180 | 41.3 | 67.8 | SR338 | 60.3 | 74 |
| SR184 | 48.1 | 69.3 | SR340 | −29.7 | 57.7 |
| SR194 | −27.9 | 49.9 | SR345 | 44.6 | 65.3 |

TABLE 10
Dose response knockdown of SLC10A1 mRNA expression
by backbone modified siRNA duplexes

| | | Maximum |
|---|---|---|
| | | measured gene |
| Duplex ID | IC₅₀ (nM) | knockdown (%) |
| SR006 | 2.9 | 86.0 |
| SR009 | 2.3 | 77.8 |
| SR010 | 2.9 | 85.5 |
| SR015 | 3.2 | 88.8 |
| SR052 | 0.5 | 87.8 |
| SR056 | 5.7 | 86.5 |
| SR060 | 1.4 | 94.4 |
| SR072 | 3.2 | 89.3 |
| SR103 | 1.7 | 92.0 |
| SR111 | 1.6 | 91.1 |
| SR122 | 2.0 | 94.5 |
| SR130 | 4.9 | 88.4 |
| SR131 | 1.7 | 93.9 |
| SR134 | 3.0 | 89.0 |
| SR140 | 4.2 | 88.5 |
| SR142 | 2.0 | 91.1 |
| SR145 | 2.2 | 87.9 |
| SR149 | 2.1 | 86.0 |
| SR155 | 3.6 | 91.8 |
| SR158 | 3.4 | 86.8 |
| SR167 | 2.7 | 90.2 |
| SR178 | 2.3 | 80.7 |
| SR187 | 1.7 | 88.3 |
| SR189 | 2.1 | 86.1 |
| SR204 | 2.0 | 89.0 |
| SR212 | 2.6 | 89.7 |
| SR222 | 2.7 | 90.5 |
| SR232 | 5.0 | 86.5 |
| SR241 | 3.6 | 87.5 |
| SR244 | 2.5 | 75.0 |
| SR264 | 2.1 | 90.6 |
| SR268 | 3.1 | 84.7 |
| SR271 | 1.5 | 87.2 |
| SR274 | 1.4 | 89.0 |
| SR275 | 3.8 | 89.7 |
| SR288 | 2.1 | 94.4 |
| SR303 | 3.1 | 89.2 |
| SR320 | 1.6 | 93.4 |
| SR328 | 5.0 | 88.1 |
| SR334 | 3.2 | 90.1 |
| SR336 | 3.2 | 84.9 |
| SR399 | 2.2 | 94.1 |
| SR456 | 4.2 | 91.0 |
| SR532 | 5.6 | 88.4 |
| SR540 | 5.6 | 88.8 |
| SR646 | 2.8 | 95.8 |
| SR676 | 10.9 | 84.0 |
| SR698 | 2.0 | 93.4 |
| SR702 | 2.5 | 94.4 |

### Example 4: Bile Acid Uptake Inhibition In Vitro

Primary human hepatocytes were treated with siRNA during plating and utilized ASGPR-mediated uptake into the cells. Seventy-two hours post treatment, cells were evaluated for bile acid uptake. Briefly, 50 μM Taurocholic acid (TCA) were added to the cells. Cells were treated for 30 minutes at 37° ° C. in a humidified incubator. TCA was then aspirated, and cells were washed four times with ice cold Dulbecco's phosphate buffered saline (PBS) containing calcium and magnesium. After the fourth wash, cells were lysed in 70% acetonitrile and frozen at −80° C. Samples were processed and TCA concentrations were quantified using RapidFire mass spectrometry. Table 11 shows the average bile acid transport inhibition in two primary human hepatocyte donors.

TABLE 11
Average bile acid transport inhibition
in primary human hepatocytes

| | IC₅₀ (nM) | IC₅₀ (nM) |
|---|---|---|
| Duplex ID | Donor I | Donor M |
| SR060 | 1.05 | — |
| SR103 | 0.55 | 4.95 |
| SR122 | 0.83 | 7.73 |
| SR131 | 0.83 | — |
| SR264 | 0.59 | — |
| SR320 | 1.05 | 6.77 |

### Example 5: Pharmacology of siRNAs in Non-Human Primate (NHP) Studies

siRNAs described herein can have long-acting in vivo pharmacology when studied in non-human primates (NHPs).

An exemplary siRNA, duplex ID SR122, was administered as a single dose at either 3 mg/kg (3 mpk) or 10 mg/kg (10 mpk) to cynomolgus monkeys.

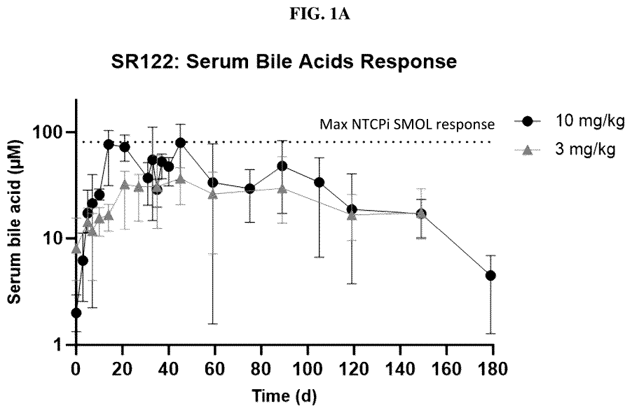

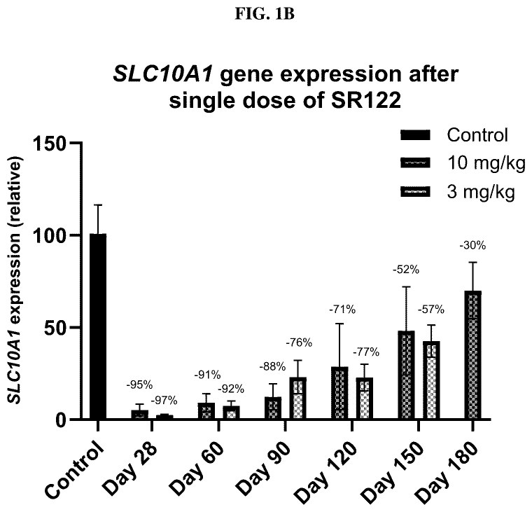

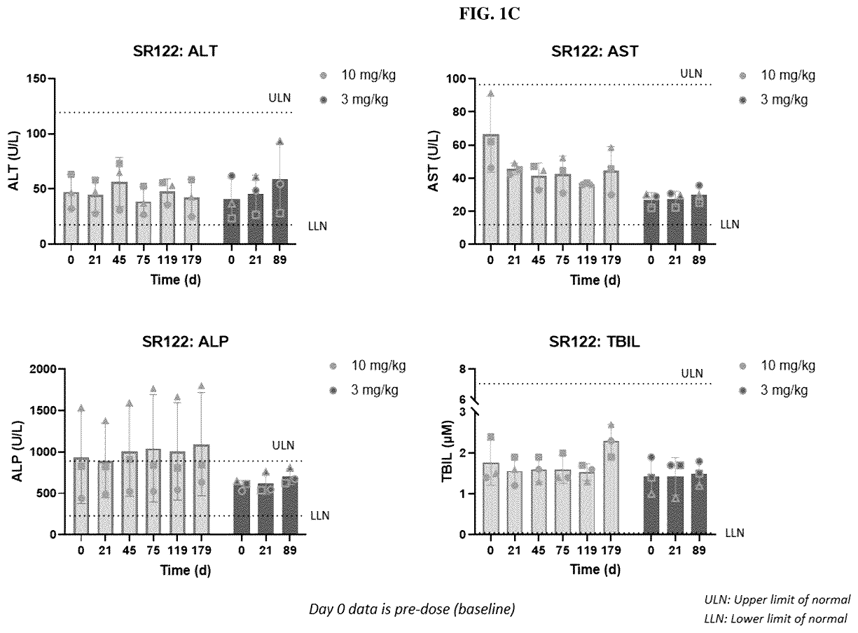

FIG. 1A illustrates that, as an average of three animals receiving SR122, SR122 shows excellent and durable evidence of target engagement for both doses, including as studied for 120 days for the 10 mpk dose. An alternative mechanism of inhibition of sodium taurocholate cotransporting polypeptide (NTCPi) by a small molecule (“SMOL”) is provided for comparison.

FIG. 1B illustrates that, as an average of three animals receiving SR122 and three control animals, liver biopsy data demonstrated robust gene knockdown at both 3 and 10 mg/kg doses, including at Day 150 for the latter dose.

FIG. 1C illustrates that liver function tests (LFTs) over the course of the 10 mg/kg study remain unchanged from baseline values for ALT, ALP, AST, and TBIL, which are supportive of target and drug candidate safety.

### Example 6: In Vivo Knockdown of mNTCP

Duplex ID SR319 has been identified as a mouse NTCP-targeting surrogate siRNA for in vivo studies.

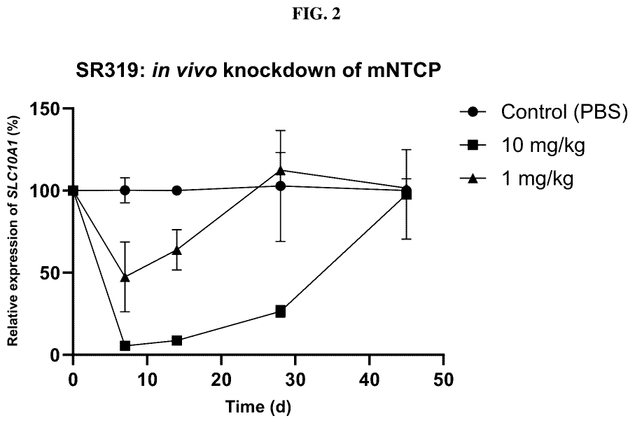

As illustrated in FIG. 2, an in vivo proof of principle target engagement study for gene knockdown has shown robust (>90%), durable, and dose responsive mRNA knockdown in mice by SR319.

SR319 was also well tolerated by all animals (n=16) through study completion at day 45.

A 10 mg/kg dose of SR319 over 14 days (SC injection at day −7, BDL at day 0, sacrifice at Day 7) was selected for the bile duct ligation model of Example 6.

### Example 7: Bile-Duct Ligation (BDL) Cholestasis Model

The bile-duct ligation (BDL) mouse model is well characterized, widely used and recapitulates fundamental aspects of human cholestatic liver injury allowing evaluation of acute hepatoprotective effects. This model has been used to characterize the effects of bulevirtide (an inhibitor of NTCP) and UDCA. These studies can be used to demonstrate that reducing bile acid uptake by NTCP inhibition (SMOL) or knock down (siRNA) will ameliorate the hepatocyte injury and secondary inflammatory, fibrotic response induced in an acute obstructive cholestasis model, including by showing: reduction of liver injury markers; and reduction of inflammatory, fibrotic, proliferative, and FXR responses.

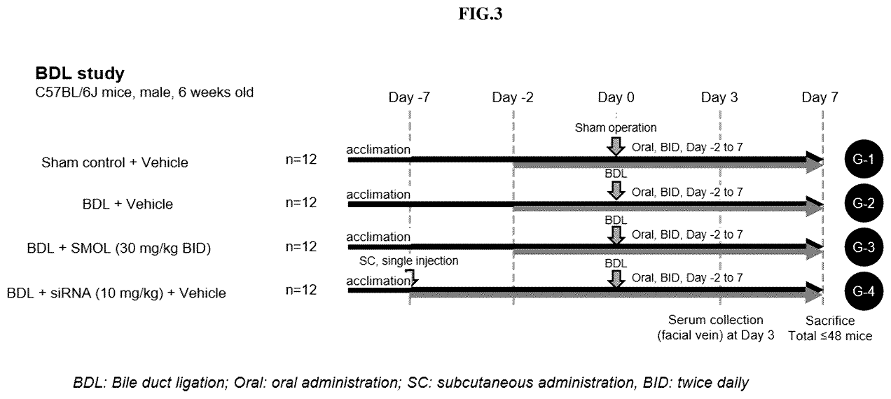

A 7-day BDL mouse model was chosen to assess both hepatoprotective effects of an exemplary siRNA and an exemplary small molecule in the acute phase as well as effects on inflammation and fibrosis that start occurring after day 5. The study design is illustrated in FIG. 3.

Prophylactic treatment was initiated at day −7 for the single 10 mg/kg dose of an exemplary siRNA (duplex ID=SR319) to allow for 7 days of Slc10a1 knockdown prior to the bile duct ligation procedure and at day −2 for an exemplary small molecule (“SMOL”) inhibitor of NTCP. SMOL was dosed BID at 30 mg/kg/dose for 9 days.

Bile duct ligation was performed at day 0, and animals were sacrificed at day 7. Serum was collected at day 3 and 7.

Endpoints for this study included:
Liver injury markers (ALP, ALT, T-bilirubin); and
Inflammatory, fibrotic, proliferative and FXR response markers (gene expression) (Tnfa, Mcp-1, TGFb, Col1a1, Foxmb1, Krt7, Shp-1, Mdr2, Ostb).

As shown herein, treatment according to the present example has demonstrated the efficacy of certain exemplary treatments, including showing that reducing bile acid uptake by NTCP inhibition using an exemplary small molecule (SMOL) or knock down using an exemplary siRNA (duplex ID=SR319) ameliorated the hepatocyte injury and secondary inflammatory, fibrotic effects in the BDL acute, complete obstructive cholestasis model. In particular, the present example illustrates: reduction of ALP and ALT; reduction of inflammatory and fibrotic gene response; reduction of proliferative gene response; and reduction in FXR signaling.

### Target Engagement

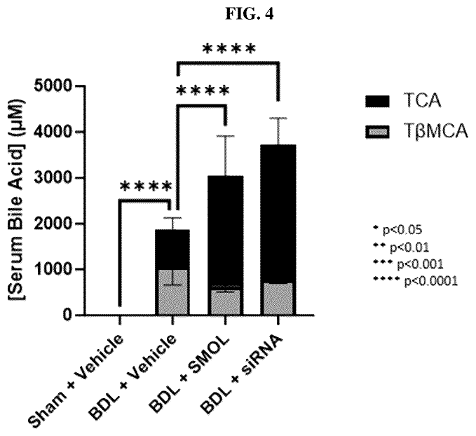

Knockdown or inhibition of NTCP will prevent uptake of bile acids by the hepatocyte and as a result, bile acids in the serum will be increased. As illustrated in FIG. 4, inhibition of NTCP with an exemplary small molecule (SMOL) and knockdown with siRNA (SR319) results in an increase in taurocholic acid (TCA) in the serum on day 7, indicative of target engagement. Response was comparable to data reported for bulevirtide (Slijepcevic et al., Hepatology, 2018, 68, 1057-1069).

### Liver Injury Markers

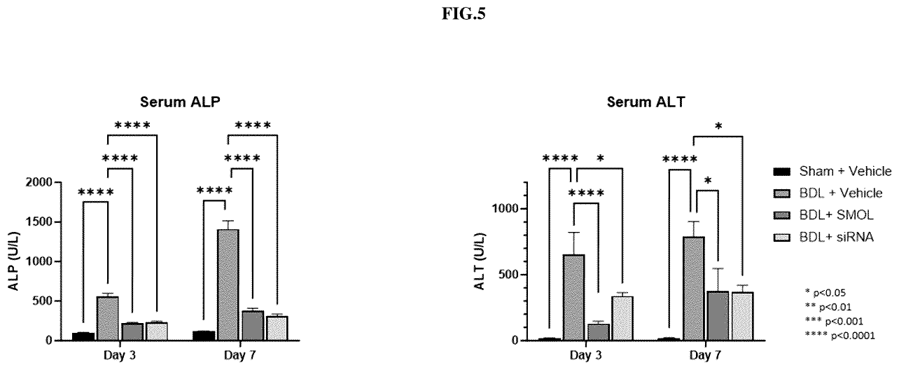

Rapidly after BDL, mice develop obstructive jaundice and cholestasis due to bile acid-induced hepatotoxicity, marked by elevated alkaline phosphatase (ALP), serum transaminase activity (ALT) and bilirubin levels. As illustrated in FIG. 5, inhibition of NTCP with an exemplary small molecule (SMOL) and siRNA (SR319) results in a clear reduction of ALP and ALT at both day 3 and day 7, indicative of a hepatoprotective function. Response was comparable to data reported for bulevirtide (Slijepcevic et al., Hepatology, 2018, 68, 1057-1069). As bilirubin is transported by OATP and thus, T-bil levels are not affected.

### Inflammation, Fibrosis, and Proliferation

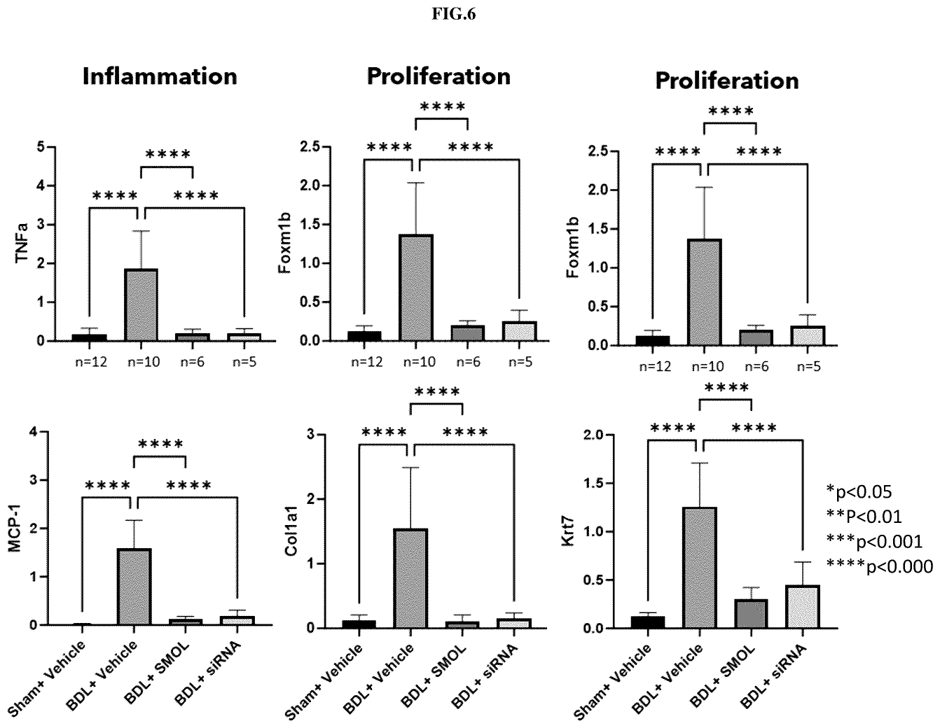

Acute hepatocyte injury in BDL is followed by infiltration of immune cells, hepatocyte and cholangiocyte proliferation and activation of stellate cells and initiation of a fibrotic response. FIG. 6 illustrates that inhibiting NTCP with an exemplary siRNA (duplex ID=SR319) or an exemplary small molecule (SMOL) reduces inflammatory (TNFa and MCP-1), fibrotic (TGF-b and Col1a1), and proliferative (Foxm1b and Krt7) markers. Reduction of inflammatory and proliferative markers gene expression was observed, consistent with an ameliorated hepatocyte injury, and reduction of inflammatory and proliferative markers gene expression was observed, consistent with an ameliorated hepatocyte injury. These data are consistent with previously published BLV-peptide results (Slijepcevic et al., Hepatology, 2018, 68, 1057-1069).

### FXR Signaling

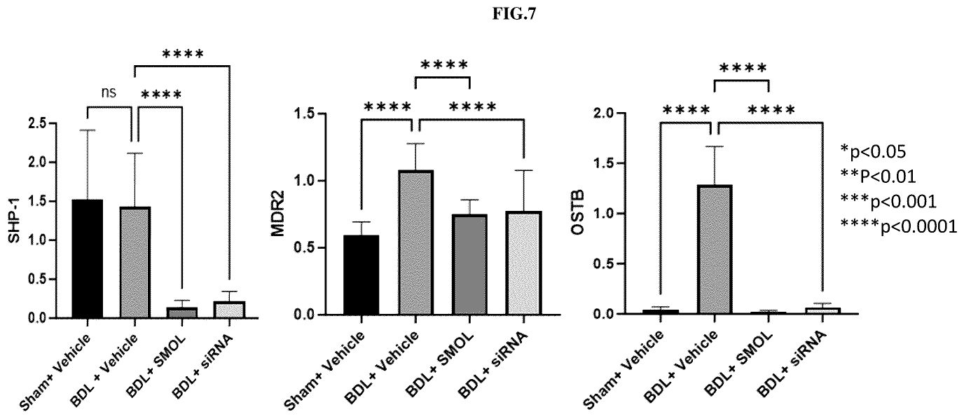

The bile acid (BA) nuclear receptor, FXR, mediates the expression of genes involved in BA synthesis, metabolism and transport that can serve as surrogate markers of intracellular BA accumulation in hepatocytes. Inhibition of NTCP results in reduced levels of FXR target genes Shp-1, Mdr2 and Ostb, indicative of lower intracellular BA levels in hepatocytes. Reduced hepatocyte FXR signaling may have a secondary benefit of reducing pruritus in humans. FIG. 7 illustrates that an exemplary siRNA (duplex ID=SR319) and an exemplary small molecule (SMOL) each lower intracellular bile acids as evidenced by reduced FXR signaling.

### EQUIVALENTS AND SCOPE

Those skilled in the art would recognize, or be able to ascertain using no more than routine experimentation, many equivalents to the specific embodiments of the invention described herein. The scope of the invention is not intended to be limited to the above Description, but rather is as set forth in the following claims:

1. A small interfering RNA (siRNA) molecule targeting a SLC10A1 mRNA transcript, comprising a double-stranded region of 17- to 30-base pairs in length that is formed between the complementary base pairs of a sense strand sequence and an antisense strand sequence, wherein the siRNA comprises a sense strand sequence selected from the group consisting of SEQ ID Nos. 1-15, 31-45 and 481-611, and/or an antisense strand sequence selected from the group consisting of SEQ ID Nos. 16-30, 46-60 and 612-742.

2. (canceled)

3. The siRNA molecule of claim 1, wherein the sequence of the siRNA molecule has 100% complementary, or a mismatch of 4 base pairs or fewer to a portion of the sequence of the SLC10A1 mRNA transcript.

4.-5. (canceled)

6. The siRNA molecule of claim 1, wherein the siRNA molecule targets a human SLC10A1 mRNA transcript.

7.-9. (canceled)

10. The siRNA molecule of claim 1, wherein the siRNA molecule comprises at least one overhang, each overhang having up to six or fewer nucleotides.

11.-16. (canceled)

17. The siRNA molecule of claim 1, wherein the siRNA comprises one or more chemical modifications, and wherein the one or more chemical modifications is 2′-O-methyl modification, 2′-fluoro modification, introduction of phosphorothioate modified nucleotides, substitution of uracil with 5-Propynyluracil, substitution of uracil with 5′-methyluridine, substitution of uracil ribose nucleotides with 4′-thioribose and substitution of uracil ribose nucleotides with deoxythymidine nucleotides, or any combinations thereof.

18.-21. (canceled)

22. The siRNA molecule of claim 17, wherein the chemical modifications include at least one 2′-O-methyl modification and/or at least one 2′-fluoro modification.

23.-24. (canceled)

25. The siRNA molecule of claim 1, wherein the siRNA is conjugated to at least one N-acetylgalactosamine (GalNAc) moiety or derivative thereof.

26. (canceled)

27. The siRNA molecule of claim 25, wherein the GalNAc moiety is conjugated to the 3′ end of the sense strand.

28.-29. (canceled)

30. The siRNA molecule of claim 17, wherein the siRNA comprises a sense strand sequence selected from the group consisting of SEQ ID Nos. 61-270 and 743-873, and/or an antisense strand sequence selected from the group consisting of SEQ ID Nos. 271-480 and 874-1004.

31. The siRNA molecule of claim 30, comprising a pair of sense and antisense strand sequences selected from the group consisting of the pairs of SEQ ID NOs. 95 and 305; SEQ ID NOs. 110 and 320; SEQ ID NOs. 121 and 331; SEQ ID NOs. 125 and 335; SEQ ID NOs. 143 and 353; SEQ ID NOs. 181 and 391; SEQ ID NOs. 200 and 410; SEQ ID NOs. 215 and 425.

32. A pharmaceutical composition comprising the siRNA molecule according to claim 1, and a pharmaceutically acceptable excipient or carrier.

33.-34. (canceled)

35. A method of treating a cholestatic disorder in a patient in need thereof comprising administering to the patient a siRNA molecule targeting a human SLC10A1 mRNA transcript, comprising a pair of a sense strand sequence and an antisense strand sequence, wherein the siRNA comprises a sense strand sequence selected from the group consisting of SEQ ID Nos. 1-15, 31-45, 61-270, 481-611, and 743-873, and/or an antisense strand sequence selected from the group consisting of SEQ ID Nos. 16-30, 46-60 271-480, 612-742, and 874-1004.

36. The method of claim 35, wherein the cholestatic disorder is progressive intrahepatic familial cholestasis (PFIC), Alagille syndrome (ALGS), biliary atresia (BA), primary biliary cholangitis (PBC), primary sclerosing cholangitis (PSC), or intrahepatic cholestasis of pregnancy (ICP), or cholestasis caused by another disease or procedure, such as cholestasis after liver transplantation or cholestasis from a malignant biliary obstruction.

37. (canceled)

38. A method of treating in a patient in need comprising administering to the patient the siRNA molecule of claim 1, wherein the patient has liver damage, including inflammation, fibrosis, cirrhosis, liver failure, and hepatocellular carcinoma.

39.-42. (canceled)

43. A method of treating a disease that is associated with bile acid transporter, sodium taurocholate co-transporting polypeptide (NTCP), comprising silencing or reducing expression of a human SLC10A1 mRNA transcript with the siRNA molecule of claim 1.

44. The method of claim 43, wherein the disease is a cholestatic disorder, hepatitis B, hepatitis D, MASLD, MASH, MetALD (also known as NAFLD or NASH), diabetes, obesity, dyslipidemia, inflammatory bowel disease, or constipation inflammatory bowel disease (IBD).

45.-48. (canceled)

49. A method for reducing expression of a SLC10A1 mRNA transcript in a cell comprising administering the siRNA of claim 1.

50.-51. (canceled)

52. The siRNA molecule of claim 1 comprising a pair of a modified sense strand sequence and a modified anti-sense strand sequence listed in Table 3, Table 5 and Table 6.

53.-56. (canceled)

57. A method of treating a viral hepatitis B and/or hepatitis D disease in a subject using a siRNA targeting a human SLC10A1 mRNA transcript, comprising a pair of a sense strand sequence and an antisense strand sequence as a monotherapy or in a regimen combination with one or more antiviral agents, wherein the siRNA comprises a sense strand sequence selected from the group consisting of SEQ ID Nos. 1-15, 31-45, 61-270, 481-611, and 743-873, and/or an antisense strand sequence selected from the group consisting of SEQ ID Nos. 16-30, 46-60 271-480, 612-742, and 874-1004.

58.-70. (canceled)

71. The siRNA molecule of claim 31, comprising a sense strand sequence of SEQ ID NO. 110 and an antisense strand sequence of SEQ ID NO. 320, and wherein the siRNA comprises at least one GalNAc moiety or derivative thereof.

72.-77. (canceled)
